SEQUENCE LISTING (15 sequences)

SEQ ID NO: 1 - DNA, 3112 bp, Homo sapiens
gaccccaggtccggacaggccgagatgacgccgagccccctgttgctgctcctgctgccgccgctgctgctgggggccttcccaccggccgccgccgcccgaggccccccaaagatggcggacaaggtggtcccacggcaggtggcccggctgggccgcactgtgcggctgcagtgcccagtggagggggacccgccgccgctgaccatgtggaccaaggatggccgcaccatccacagcggctggagccgcttccgcgtgctgccgcaggggctgaaggtgaagcaggtggagcgggaggatgccggcgtgtacgtgtgcaaggccaccaacggcttcggcagccttagcgtcaactacaccctcgtcgtgctggatgacattagcccagggaaggagagcctggggcccgacagctcctctgggggtcaagaggaccccgccagccagcagtgggcacgaccgcgcttcacacagccctccaagatgaggcgccgggtgatcgcacggcccgtgggtagctccgtgcggctcaagtgcgtggccagcgggcaccctcggcccgacatcacgtggatgaaggacgaccaggccttgacgcgcccagaggccgctgagcccaggaagaagaagtggacactgagcctgaagaacctgcggccggaggacagcggcaaatacacctgccgcgtgtcgaaccgcgcgggcgccatcaacgccacctacaaggtggatgtgatccagcggacccgttccaagcccgtgctcacaggcacgcaccccgtgaacacgacggtggacttcggggggaccacgtccttccagtgcaaggtgcgcagcgacgtgaagccggtgatccagtggctgaagcgcgtggagtacggcgccgagggccgccacaactccaccatcgatgtgggcggccagaagtttgtggtgctgcccacgggtgacgtgtggtcgcggcccgacggctcctacctcaataagctgctcatcacccgtgcccgccaggacgatgcgggcatgtacatctgccttggcgccaacaccatgggctacagcttccgcagcgccttcctcaccgtgctgccagacccaaaaccgcaagggccacctgtggcctcctcgtcctcggccactagcctgccgtggcccgtggtcatcggcatcccagccggcgctgtcttcatcctgggcaccctgctcctgtggctttgccaggcccagaagaagccgtgcacccccgcgcctgcccctcccctgcctgggcaccgcccgccggggacggccctcgaccgcagcggagacaaggaccttccctcgttggccgccctcagcgctggccctggtgtggggctgtgtgaggagcatgggtctccggcagccccccagcacttactgggcccaggcccagttgctggccctaagttgtaccccaaactctacacagacatccacacacacacacacacacactctcacacacactcacacgtggagggcaaggtccaccagcacatccactatcagtgctagacggcaccgtatctgcagtgggcacgggggggccggccagacaggcagactgggaggatggaggacggagctgcagacgaaggcaggggacccatggcgaggaggaatggccagcaccccaggcagtctgtgtgtgaggcatagcccctggacacacacacacagacacacacactacctggatgcatgtatgcacacacatgcgcgcacacgtgctccctgaaggcacacgtacgcacacacgcacatgcacagatatgccgcctgggcacacagataagctgcccaaatgcacgcacacgcacagagacatgccagaacatacaaggacatgctgcctgaacatacacacgcacacccatgcgcagatgtgctgcctggacacacacacacacacggatatgctgtctggacgcacacacgtgcagatatggtatccggacacacacgtgcacagatatgctgcctggacacacagataatgctgccttgacacacacatgcacggatattgcctggacacacacacacacacgcgtgcacagatatgctgtctggacaggcacacacatgcagatatgctgcctggacacacacttccagacacacgtgcacaggcgcagatatgctgcctggacacacgcagatatgctgtctagtcacacacacacgcagacatgctgtccggacacacacacgcatgcacagatatgctgtccggacacacacacgcacgcagatatgctgcctggacacacacacagataatgctgcctcaacactcacacacgtgcagatattgcctggacacacacatgtgcacagatatgctgtctggacatgcacacacgtgcagatatgctgtccggatacacacgcacgcacacatgcagatatgctgcctgggcacacacttccggacacacatgcacacacaggtgcagatatgctgcctggacacacgcagactgacgtgcttttgggagggtgtgccgtgaagcctgcagtacgtgtgccgtgaggctcatagttgatgagggactttccctgctccaccgtcactcccccaactctgcccgcctctgtccccgcctcagtccccgcctccatccccgcctctgtcccctggccttggcggctatttttgccacctgccttgggtgcccaggagtcccctactgctgtgggctggggttgggggcacagcagccccaagcctgagaggctggagcccatggctagtggctcatccccactgcattctccccctgacacagagaaggggccttggtatttatatttaagaaatgaagataatattaataatgatggaaggaagactgggttgcagggactgtggtctctcctggggcccgggacccgcctggtctttcagccatgctgatgaccacaccccgtccaggccagacaccaccccccaccccactgtcgtggtggccccagatctctgtaattttatgtagagtttgagctgaagccccgtatatttaatttattttgttaaacatgaaagtgcatcctttccctccaaaaaaaaaaaaaaaaaaaaaaa

SEQ ID NO: 2 - protein, 504 aa, Homo sapiens [sequence omitted]

SEQ ID NO: 3 - protein, 802 aa, Homo sapiens
MRLLLALLGVLLSVPGPPVLSLEASEEVELEPCLAPSLEQQEQELTVALGQPVRLCCGRAERGGHWYKEGSRLAPAGRVRGWRGRLEIASFLPEDAGRYLCLARGSMIVLQNLTLITGDSLTSSNDDEDPKSHRDPSNRHSYPQQAPYWTHPQRMEKKLHAVPAGNTVKFRCPAAGNPTPTIRWLKDGQAFHGENRIGGIRLRHQHWSLVMESVVPSDRGTYTCLVENAVGSIRYNYLLDVLERSPHRPILQAGLPANTTAVVGSDVELLCKVYSDAQPHIQWLKHIVINGSSFGADGFPYVQVLKTADINSSEVEVLYLRNVSAEDAGEYTCLAGNSIGLSYQSAWLTVLPEEDPTWTAAAPEARYTDIILYASGSLALAVLLLLAGLYRGQALHGRHPRPPATVQKLSRFPLARQFSLESGSSGKSSSSLVRGVRLSSSGPALLAGLVSLDLPLDPLWEFPRDRLVLGKPLGEGCFGQVVRAEAFGMDPARPDQASTVAVKMLKDNASDKDLADLVSEMEVMKLIGRHKNIINLLGVCTQEGPLYVIVECAAKGNLREFLRARRPPGPDLSPDGPRSSEGPLSFPVLVSCAYQVARGMQYLESRKCIHRDLAARNVLVTEDNVMKIADFGLARGVHHIDYYKKTSNGRLPVKWMAPEALFDRVYTHQSDVWSFGILLWEIFTLGGSPYPGIPVEELFSLLREGHRMDRPPHCPPELYGLMRECWHAAPSQRPTFKQLVEALDKVLLAVSEEYLDLRLTFGPYSPSGGDASSTCSSSDSVFSHDPLPLGSSSFPFGSGVQT

SEQ ID NO: 4 - DNA, 39 bp, Homo sapiens
gcagcacatatggcccgaggccccccaaagatggcggac

SEQ ID NO: 5 - DNA, 46 bp, Homo sapiens
gcagcaggtaccttaagtggccgaggacgaggaggccacaggtggc

SEQ ID NO: 6 - DNA, 48 bp, Homo sapiens
gcagcaggatccgccatcatgacgccgagccccctgttgctgctcctg

SEQ ID NO: 7 - DNA, 46 bp, Homo sapiens
gcagcaggtaccttagcactgatagtggatgtgctggtggaccttg

SEQ ID NO: 8 - DNA, 42 bp, Homo sapiens
gcagcacatatgcgccatcaacgccacctacaaggtggatgt

SEQ ID NO: 9 - DNA, 46 bp, Homo sapiens; identical to SEQ ID NO: 7

SEQ ID NO: 10 - DNA, 42 bp, Homo sapiens; identical to SEQ ID NO: 8

SEQ ID NO: 11 - DNA, 46 bp, Homo sapiens; identical to SEQ ID NO: 5

SEQ ID NO: 12 - DNA, 733 bp, Homo sapiens
gggatccggagcccaaatcttctgacaaaactcacacatgcccaccgtgcccagcacctgaattcgagggtgcaccgtcagtcttcctcttccccccaaaacccaaggacaccctcatgatctcccggactcctgaggtcacatgcgtggtggtggacgtaagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaacccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccgcgactctagaggat

SEQ ID NO: 13 - DNA, 486 bp, Homo sapiens
  misc_feature (410): n equals a, t, g, or c
gcggccgcgaccccaggtccggacaggccgagatgacgccgagccccctgttgctgctcctgctgccgccgctgctgctgggggccttcccaccggccgccgccgcccgaggccccccaaagatggcggacaaggtggtcccacggcaggtggcccggctgggccgcactgtgcggctgcagtgcccagtggagggggacccgccgccgctgaccatgtggaccaaggatggccgcaccatccacagcggctggagccgcttccgcgtgctgccgcaggggctgaaggtgaagcaggtggagcgggaggatgccggcgtgtacgtgtgcaaggccaccaacggcttcggcagccttagcgtcaactacaccctcgtcgtgctggatgacattagcccagggaaggagagncctgggcccgacagctccttctggggtcaagaggaccccgccagccagcagtgggcacgaccgcgcttcacacagc

SEQ ID NO: 14 - DNA, 46 bp, Homo sapiens
gcagcaggtaccttaagtggccgaggagcaggaggccacaggtggc

SEQ ID NO: 15 - DNA, 46 bp, Homo sapiens; identical to SEQ ID NO: 14

(19) United States
(12) Patent Application Publication
(10) Pub. No.: US 2001/0016335 A1
(43) Pub. Date: Aug. 23, 2001

(54) Fibroblast growth factor receptor-5

(76) Inventors: Paul E. Young, Gaithersburg, MD (US); Steven M. Ruben, Olney, MD (US)

Correspondence Address: HUMAN GENOME SCIENCES INC, 9410 KEY WEST AVENUE, ROCKVILLE, MD, 20850

(21) Appl. No.: 09/758,386

(22) Filed: Jan. 12, 2001

Related U.S. Application Data
(63) Continuation of application No. 09/293,182, filed on Apr. 16, 1999, now abandoned.
(60) Provisional application No. 60/105,465, filed on Oct. 23, 1998.

(51) Int. Cl. A61K 38/00; A61K 31/70; C12P 21/06; C07K 1/00; C07K 14/00; C07K 17/00

(52) U.S. Cl. 435/69.1; 435/320.1; 514/12; 514/44; 530/350

(57) ABSTRACT

The present invention relates to fibroblast growth factor receptor-5, a novel member of the fibroblast growth factor receptor family. The invention provides isolated nucleic acid molecules encoding human FGFR5 receptors. FGFR5 polypeptides are also provided, as are vectors, host cells and recombinant methods for producing the same. The invention further relates to screening methods for identifying agonists and antagonists of FGFR5 receptor activity. Also provided are diagnostic methods for detecting disease states related to the aberrant expression of FGFR5 receptors. Further provided are therapeutic methods for treating disease states including, but not limited to, defects in wound healing, mucositis, defects in angiogenesis, ischemia, host defense dysfunction, endocrine dysfunction, disorders in immune function, and/or disorders in insulin secretion.

This application claims benefit under 35 U.S.C. § 119(e) of the filing date of copending U.S. Provisional Application Serial No. 60/105,465, filed on Oct. 23, 1998, which is incorporated herein by reference in its entirety.

### FIELD OF THE INVENTION

The present invention relates to a novel member of the fibroblast growth factor receptor (FGFR) family. More specifically, the present invention relates to the discovery, identification and characterization of nucleotides that encode fibroblast growth factor receptor-5, a receptor having homology to the human fibroblast growth factor receptor-4. The invention encompasses FGFR-5 polynucleotides, host cell expression systems, FGFR-5 fusion proteins, antibodies to FGFR-5, agonists and antagonists of FGFR-5, and other compounds that modulate FGFR-5 gene expression or FGFR-5 activity, that can be used for the diagnosis, drug screening, and treatment or prevention of disorders which include, but are not limited to, wound healing, mucositis, angiogenesis, immune function, endocrine function, and insulin secretion.

### BACKGROUND OF THE INVENTION

Fibroblast growth factors (FGFs) are potentially the largest family of growth factors involved in tissue repair and regeneration. Members of this family are strong mitogens, capable of promoting the growth of mesodermal and ectodermal cells. FGFs are angiogenic, and induce the production of blood vessels. Furthermore, members of this family are involved in the proliferation of endothelial cells, neurite outgrowth, and are thought to play an important role in the early stages of embryonic development. Depending upon the cell types, FGFs can support cell survival and either induce or inhibit cell differentiation (Partanen, J., et al., EMBO Journal, 10:1347-1354 (1991)). FGFs are known to exert effects by binding to high affinity FGF receptor tyrosine kinases on the cell surface of particular cell types which are responsive to this binding (Johnson, D. E. and Williams, L. T., Advances in Cancer Res., 60:1-41 (1993)).

There are currently four fibroblast growth factor receptors characterized, which are designated FGFR-1, FGFR-2, FGFR-3, and FGFR-4, named respectively for the order in which they have been discovered and subsequently characterized. Splice variants of all four members of this family of receptors exist, leading to different isoforms of these receptors with subsequent differing ligand specificity. The receptor specificity is determined by the alternative splicing, as well as by sequence differences in both the fibroblast growth factors as well as the fibroblast growth factor receptors. (Omitz, D. M., et al., J. Biol. Chem., 271:15292-15297, (1996)).

All four members of the FGF receptor family share a common structual arrangement, which consists of kinase 1 and kinase 2 intracellular domains, followed by a transmembrane domain, two extracellular immunogloulin like domains (Ig domains II and III), an acid box region consisting of eight consecutive acidic residues, a third extracellular immunoglobulin domain (Ig domain I), and a hydrophobic leader sequence (Johnson, D. E. and Williams, L. T., Advances in Cancer Res., 60:1-41 (1993)). There are many characterized variants of this structural arrangement, which include, for example, a secreted form of FGFR 1 consisting of the Ig domain I, several variants of FGFR1 and FGFR 2 which lack the Ig domain I, several variants of FGFR1 and FGFR2 which contain alternative sequences for the second half of Ig domain III, and two FGFR2 variant forms which contain unique C-tail domains (Johnson, D. E. and Williams, L. T., Advances in Cancer Res., 60:1-41 (1993)).

The Ig domains are characterized by three main features: (i) the presence of two characteristic cysteine residues in each domain; (ii) the presence of a consensus tryptophan residue 11 or 12 amino acids on the COOH-terminal side of the first cysteine residue in each Ig domain; and (iii) the presence of the consensus sequence, DXGXYXC, on the NH sub 2-terminal side of the second cysteine residue. The last feature is modified in the cases of the soluble receptor proteins, and substituted with an equivalently sized sequence.

Members of the FGF family play important roles in promoting the growth of mesodermal and ectodermal cells, promoting the growth of endothelial cells and angiogenesis, and are thought to play an important role in the early stages of embryonic development. Isolated and purified FGFR5 receptors and fragments, and the DNA sequences encoding such polypeptides, will greatly enhance the understanding of the functions of FGFs, and consequently many of the mechanisms of disease and tissue repair involving FGFs. Furthermore, such FGFR5 polypeptides will aid in the diagnosis and treatment of diseases associated with FGF activity, such as wound healing, cancer, mucositis, and arthritis, to name a few. The present invention fulfills these and other needs.

### SUMMARY OF THE INVENTION

The present invention provides isolated nucleic acid molecules comprising polynucleotides encoding a FGFR-5 receptor having the amino acid sequence shown in FIGS. 1A-C (SEQ ID NO:2) or the amino acid sequence encoded by the cDNA clone encoding the FGFR5 receptor deposited in a vector with the American Type Culture Collection (“ATCC”) on Oct. 27, 1998, and assigned ATCC Deposit No. 203382. The ATCC is located at 10801 University Boulevard, Manassas, Va. 20110-2209. The present invention also relates to recombinant vectors, which include the isolated nucleic acid molecules of the present invention, and to host cells containing the recombinant vectors, as well as to methods of making such vectors and host cells and for using them or other genetically modified host cells to produce FGFR5 polypeptides (including fragments, variants, derivatives, and analogs thereof) by recombinant techniques.

The invention further provides isolated FGFR5 polypeptides having amino acid sequences encoded by the polynucleotides described herein.

The present invention also provides a screening method for identifying compounds capable of eliciting a cellular response induced by FGFR5, which involves contacting cells which express FGFR5 with the candidate compound, assaying a cellular response (e.g., ion flux, hydolysis of polyphosphoinositides), and comparing the cellular response to a standard cellular response, the standard being assayed in absence of the candidate compound; whereby, an increased cellular response over the standard indicates that the compound is an agonist.

The present invention also provides a screening method for identifying compounds capable of enhancing or inhibiting a cellular response induced by FGFR5 receptors, which involves contacting cells which express FGFR5 receptors with the candidate compound in the presence of a FGFR5 agonist (e.g., a member of the FGF ligand family), assaying a cellular response (e.g., ion flux, such as, Ca⁺² flux, or hydolysis of polyphosphoinositides), and comparing the cellular response to a standard cellular response, the standard being assayed when contact is made between the agonist and FGFR5 or when FGFR5 is exposed to the stimulus, in absence of the candidate compound; whereby, an increased cellular response over the standard indicates that the compound is an agonist and a decreased cellular response over the standard indicates that the compound is an antagonist.

In another embodiment, a screening assay for agonists and antagonists is provided which involves determining the effect a candidate compound has on the binding of cellular ligands (e.g., a member of the FGF family) to FGFR5. In particular, the method involves contacting FGFR5 with a ligand and a candidate compound and determining whether ligand binding to the FGFR5 receptors is increased or decreased due to the presence of the candidate compound.

The invention further provides a diagnostic method useful during diagnosis or prognosis of disease states resulting from aberrant cell growth, or aberrant cell secretion, activation, survival, migration, differentiation and/or proliferation, due to alterations in FGFR5 coding sequences and/or receptor expression.

An additional embodiment of the invention is related to a method for treating an individual in need of an increased level of FGFR5 activity in the body comprising administering to such an individual a composition comprising a therapeutically effective amount of FGFR5 polypeptides or polynucleotides of the invention or a FGFR5 agonist.

A still further embodiment of the invention is related to a method for treating an individual in need of a decreased level of a FGFR5 receptor activity in the body comprising, administering to such an individual a composition comprising a therapeutically effective amount of FGFR5 polypeptides or polynucleotides of the invention a FGFR5 antagonist.

The invention additionally provides soluble forms of the polypeptides of the present invention. Soluble peptides are defined by amino acid sequences wherein the sequence comprises the polypeptide sequence lacking a transmembrane domain (e.g., FGFR5 polypeptide fragments corresponding to intracellular and/or extracellular domains). Such soluble forms of the FGFR5 receptor are useful as antagonists of the membrane bound forms of the receptor.

### BRIEF DESCRIPTION OF THE FIGURES

FIGS. 1A-C show the nucleotide sequence (SEQ ID NO:1) and deduced amino acid sequence (SEQ ID NO:2) of the FGFR5 receptor. The deduced complete amino acid sequence includes 504 amino acid residues and has a deduced molecular weight of about 54,530 Da. The predicted domains of the FGFR5 polypeptide are: the leader sequence (amino acid residues Met-1 to Ala-24 of FIGS. 1A-C (SEQ ID NO:2)); the extracellular immunoglobulin domain I (amino acid residues from about Ala-25 to about Leu-117 of FIGS. 1A-C (SEQ ID NO:2)); the acid box domain (amino acid residues from about Asp-118 to about Gln-143 of FIGS. 1A-C (SEQ ID NO:2)); the extracellular immunoglobulin domain II (amino acid residues from about Trp-144 to about Ile-239 of FIGS. 1A-C (SEQ ID NO:2)); the extracellular immunoglobulin domain III (amino acid residues from about Gln-240 to about Pro-357 of FIGS. 1A-C (SEQ ID NO:2)); the membrane proximal domain (amino acid residues from about Asp-358 to about Thr-373 of FIGS. 1A-C (SEQ ID NO:2)); the transmembrane domain (amino acid residues from about Ser-374 to about Lys-403 of FIGS. 1A-C (SEQ ID NO:2)); and the intracellular (or juxtamembrane) domain (amino acid residues from about Lys-404 to about Cys-504 of FIGS. 1A-C (SEQ ID NO:2)). The transmembrane domain is underscored.

FIGS. 2A-B show the regions of similarity between the amino acid sequences of the FGFR5 receptor protein of FIGS. 1A-C (labeled FGFR5.prot; SEQ ID NO:2) and human FGFR4 receptor protein (SEQ ID NO:3) which is labeled “FGFR4prot” (GenBank Accession Number 182570 (L03840)). Identical amino acid residues between FGFR5 and FGFR4 are shaded.

FIG. 3 and FIGS. 4A-D show a structural analysis of the FGFR5 receptor amino acid sequence of FIGS. 1A-C (SEQ ID NO:2), generated using the default parameters of the recited computer programs. Alpha, beta, turn and coil regions; hydrophilicity and hydrophobicity; amphipathic regions; flexible regions; antigenic index and surface probability are shown. In the “Antigenic Index—Jameson-Wolf” graph, amino acid residues: from Ala-23 to Pro-37; from Gln-39 to Arg-48; from Cys-51 to Pro-59, from Met-62 to Arg-76; from Pro-81 to Tyr-97; from Ala-101 to Gly-104; from Asp-119 to Leu-170; from Gly-176 to Trp-204; from Lys-209 to Gly-228; from Val-238 to Val-247; from Asp-259 to Gly-262; from Cys-268 to Lys-275; from Lys-282 to Lys-302; from Pro-307 to Leu-320; from Thr-326 to Gly-334; from Leu-356 to Leu-375; from Ala-401 to Leu-435; from Ala-440 to Gly-443; from Leu-446 to Ala-455; from Pro-462 to Tyr-475; and from His-483 to Val-496 as depicted in FIGS. 1A-C (SEQ ID NO:2) correspond to predicted antigenic regions of the FGFR5 receptor protein.

### DETAILED DESCRIPTION OF THE INVENTION

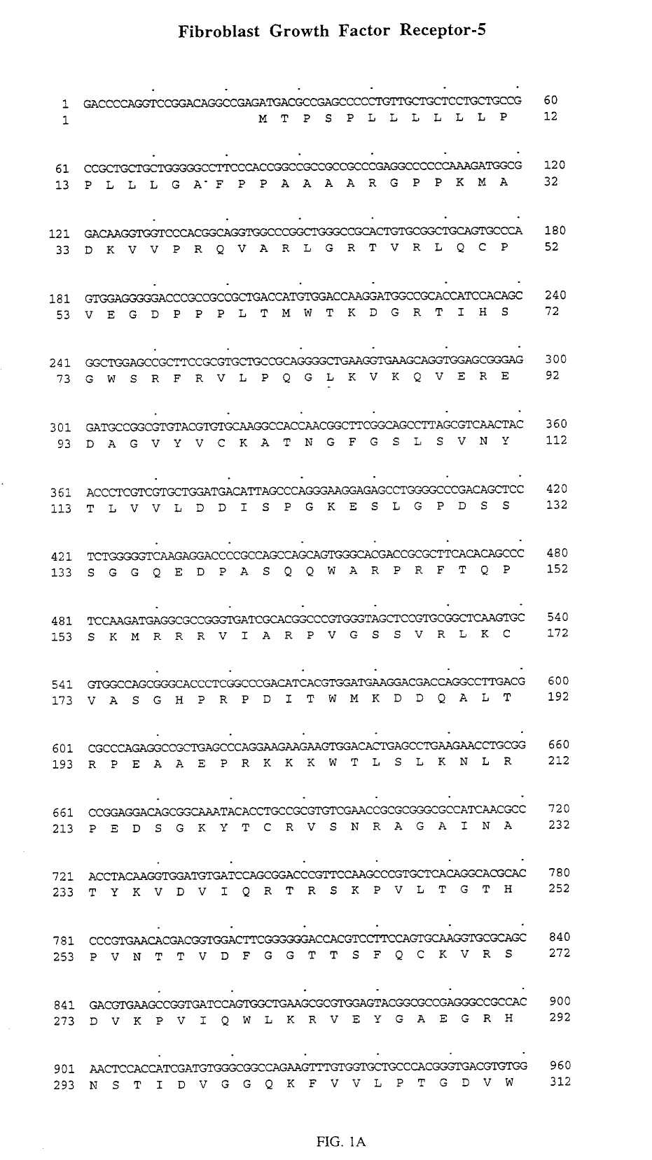

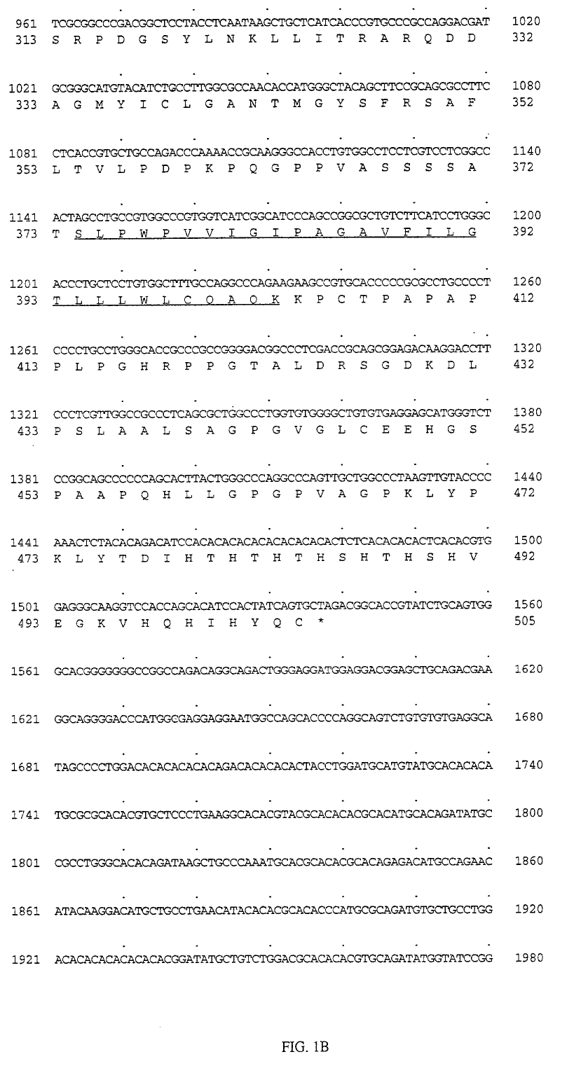

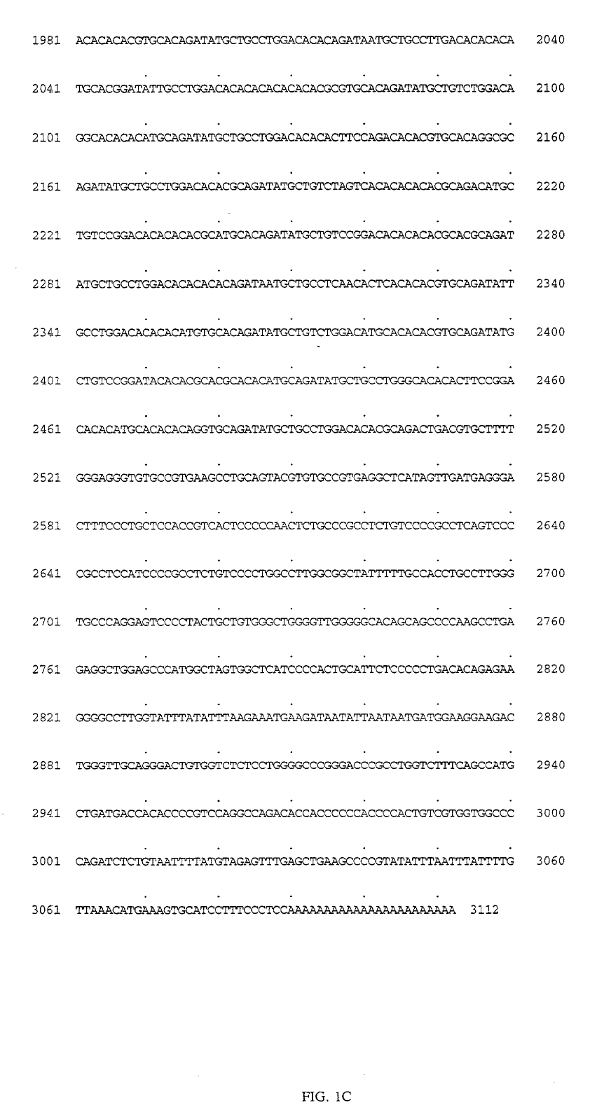

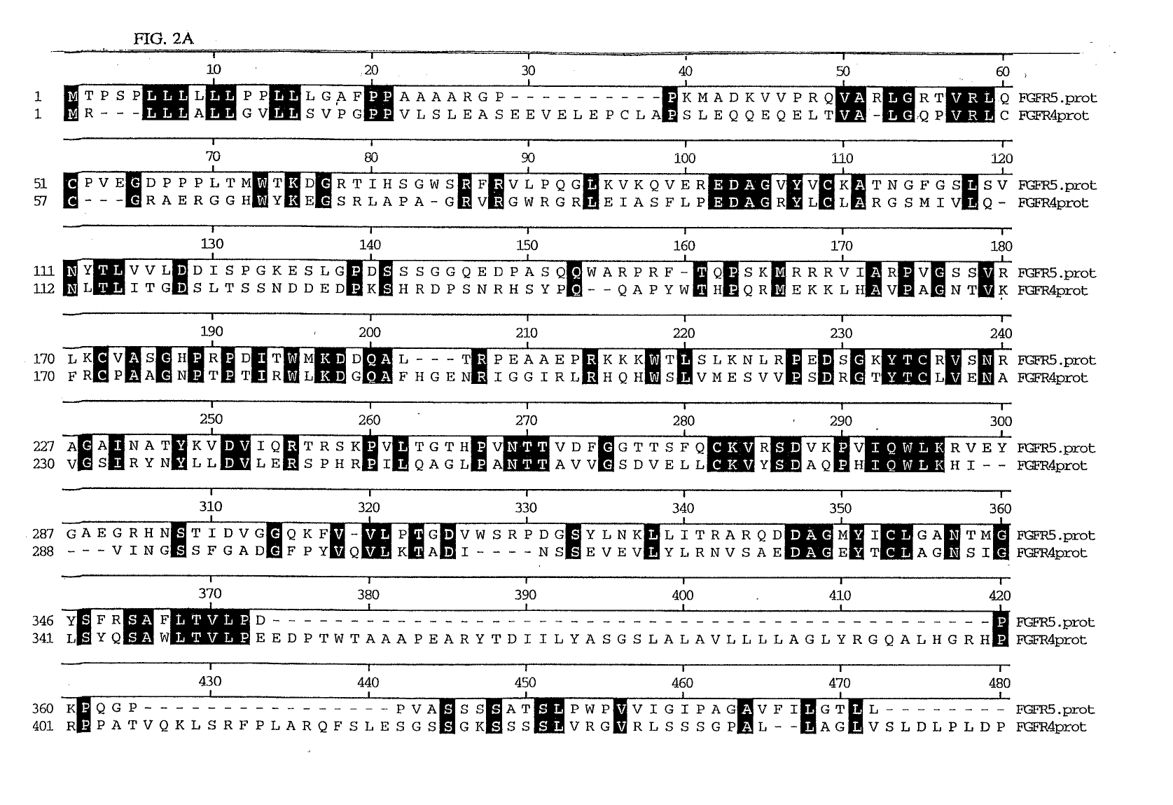

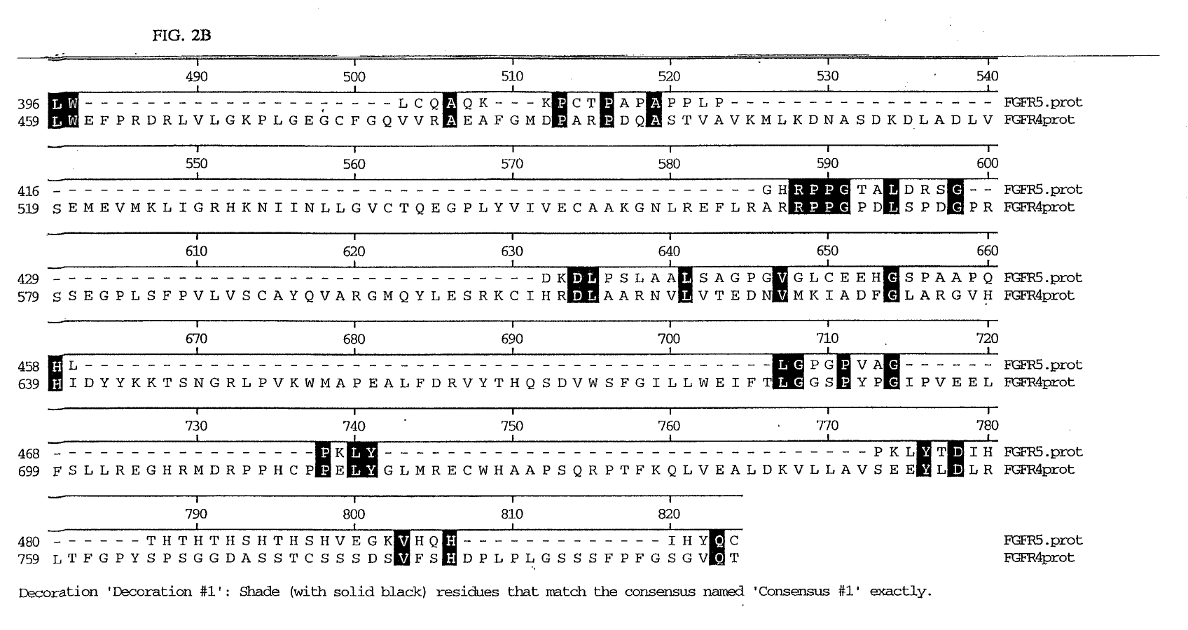

The present invention provides isolated nucleic acid molecules comprising polynucleotides encoding a FGFR-5 polypeptide (FIGS. 1A-C (SEQ ID NO:2)), a portion of the amino acid sequence of which was determined by sequencing a cloned cDNA (Clone HHERA91). For further reference, refer to copending U.S. Provisional Application Serial No. 60/105,465, filed on Oct. 23, 1998, which is incorporated herein by reference in its entirety. The FGFR5 protein shown in FIGS. 1A-C shares sequence homology with human FGFR4 (FIGS. 2A-B (SEQ ID NO:3)). A portion of the nucleotide sequence shown in FIGS. 1A-C (SEQ ID NO:1) was obtained by sequencing a cDNA clone (Clone HHERA91). On Oct. 27, 1998, the plasmid corresponding to this clone was deposited with the American Type Culture Collection, 10801 University Blvd, Manassas, Va., 20110-2209, and was assigned accession number 203382. The deposited cDNA is contained in the pCMVSport 3.0 plasmid (Life Technologies, Gaithersburg, Md.).

As used herein, “FGFR5 protein”, “FGFR5 receptor”, “receptor protein”, “FGFR5”, and “FGFR5 polypeptide” refer to all polypeptides resulting from the alternate splicing of the genomic DNA sequences encoding proteins having regions of amino acid sequence identity and receptor activity which correspond to the protein shown in FIGS. 1A-C (SEQ ID NO:2). The FGFR5 protein shown in FIG. 1 is an example of such a receptor protein.

### Nucleic Acid Molecules

Unless otherwise indicated, all nucleotide sequences determined by sequencing a DNA molecule herein were determined using an automated DNA sequencer (such as the Model 373 from Applied Biosystems, Inc.), and all amino acid sequences of polypeptides encoded by DNA molecules determined herein were predicted by translation of a DNA sequence determined as above. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

Using the information provided herein, such as the nucleotide sequence in FIGS. 1A-C (SEQ ID NO:1), nucleic acid molecules of the present invention encoding FGFR5 polypeptides may be obtained using standard cloning and screening procedures, such as those used for cloning cDNAs using mRNA as starting material. Northern analysis has revealed expression of the FGFR5 transcript in a variety of tissues, with highest levels in the pancreas, heart, kidney, liver, fetal liver, brain, and skeletal muscle. Thus, any of these tissues or cell types provide a source of FGFR5 mRNA. Additionally, any tissue or cell source may be utilized to routinely clone FGFR5 genomic DNA using techniques known in the art. Illustrative of the invention, the nucleic acid molecule described in FIGS. 1A-C (SEQ ID NO:1) was discovered in a cDNA library derived from isolated T-cells.

The determined nucleotide sequence of the FGFR5 cDNA of FIGS. 1A-C (SEQ ID NO:1) contains an open reading frame encoding a polytopic polypeptide of about 504 amino acid residues, with 1 intracellular domain, 1 transmembrane domain, 1 membrane proximal domain, 3 extracellular immunoglobulin domains, and 1 acid-box domain intervening between the first and second extracellular immunoglobulin domains, and having a deduced molecular weight of about 54,530 Da. The FGFR5 nucleotide sequence shown in FIGS. 1A-C (SEQ ID NO:1) is predicted to be about 33% identical to the human FGFR4 amino acid sequence depicted in SEQ ED NO:3 (see FIGS. 2A-B) using the computer program “MegAlign” (see below). In addition to having homology, FGFR4 and FGFR5 share the same predicted topological organization. For example, like FGFR4, FGFR5 contains an intracellular domain, followed by a transmembrane domain, a membrane proximal domain, three extracellular immunoglobulin domains, and an acid-box domain between the first and second immunoglobulin domains. Furthermore, both FGFR4 and FGFR5 share the conserved amino acid motif observed in the extracellular immunoglobulin domains of members of this family.

Nucleic acid molecules of the present invention may be in the form of RNA, such as mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand or complementary strand.

By “isolated” nucleic acid molecule(s) is intended a nucleic acid molecule, DNA or RNA, which has been removed from its native environment. For example, recombinant DNA molecules contained in a vector are considered isolated for the purposes of the present invention. Isolated RNA molecules include in vivo or in vitro RNA transcripts of the DNA molecules of the present invention. Further examples of isolated DNA molecules include recombinant DNA molecules maintained in heterologous host cells or purified (partially or substantially) DNA molecules in solution. However, a nucleic acid contained in a clone that is a member of a mixed clone library (e.g., a genomic or cDNA library) that has not been isolated from other members of the mixed clone library (e.g., in the form of a homogeneous solution containing the clone and other members of the library) or which is contained on a chromosome preparation (e.g., a chromosome spread), is not “isolated” for the purposes of this invention. Isolated nucleic acid molecules according to the present invention may be produced naturally, recombinantly, or synthetically.

In one embodiment, nucleic acid molecules of the present invention include DNA molecules comprising an open reading frame (ORF) shown in FIGS. 1A-C (SEQ ID NO:1); and DNA molecules which comprise a sequence substantially different from those described above, but which, due to the degeneracy of the genetic code, still encode the FGFR5 receptor polypeptide shown in FIGS. 1A-C (SEQ ID NO:2). Of course, the genetic code is well known in the art. Thus, it would be routine for one skilled in the art to generate such degenerate variants.

In another embodiment, the invention provides isolated nucleic acid molecules encoding the FGFR5 polypeptide having the amino acid sequence encoded by the cDNA clone contained in the plasmid deposited as ATCC Deposit No. 203382 on Oct. 27, 1998. The invention further provides isolated nucleic acid molecules having the nucleotide sequences shown in FIGS. 1A-C (SEQ ID NO:1), the nucleotide sequence of the cDNA contained in the above-described deposited clone (clone HHERA91); or nucleic acid molecules having a sequence complementary to one of the above sequences. Such isolated molecules, particularly DNA molecules, have uses that include, but are not limited to, probes for gene mapping by in situ hybridization with chromosomes, and for detecting expression of the FGFR5 genes of the present invention in human tissue, for instance, by northern blot analysis.

The present invention is further directed to fragments of the isolated nucleic acid molecules (i.e. polynucleotides) described herein. By a fragment of an isolated nucleic acid molecule having, for example, the nucleotide sequence of the deposited cDNA (clone HHERA91), a nucleotide sequence encoding the polypeptide sequence encoded by the deposited cDNA, a nucleotide sequence encoding the polypeptide sequence depicted in FIGS. 1A-C (SEQ ID NO:2), the nucleotide sequence shown in FIGS. 1A-C (SEQ ID NO:1), or the complementary strand thereto, is intended fragments at least 15 nt, and more preferably at least 20 nt, still more preferably at least 30 nt, and even more preferably, at least 40, 50, 100, 150, 200, 250, 300, 325, 350, 375, 400, 450, 500, 550, or 600 nt in length. These fragments have numerous uses which include, but are not limited to, diagnostic probes and primers as discussed herein. Of course, larger fragments, such as those of 601-995 nt in length are also useful according to the present invention as are fragments corresponding to most, if not all, of the nucleotide sequences of the deposited cDNA (clone HHERA91) or as shown in FIGS. 1A-C (SEQ ID NO:1). By a fragment at least 20 nt in length, for example, is intended fragments which include 20 or more contiguous bases from, for example, the nucleotide sequence of the deposited cDNA, or the nucleotide sequence as shown in FIGS. 1A-C (SEQ ID NO:1).

Representative examples of FGFR5 polynucleotide fragments of the invention include, for example, fragments that comprise, or alternatively, consist of, a sequence from about nucleotide 1 to 50, 51 to 100, 101 to 150, 151 to 200, 201 to 250, 251 to 300, 301 to 350, 351 to 400, 401 to 450, 451 to 500, 501 to 539, 540 to 600, 600 to 650, 651 to 700, 701 to 750, 751 to 800, 800 to 850, 851 to 900, 901 to 950, 951 to 1000, 1001 to 1050, 1051 to 1100, 1101 to 1150, 1151 to 1200, 1201 to 1250, 1251 to 1300, 1301 to 1350, 1351 to 1400, 1401 to 1450, 1451 to 1500 and/or 1501 to 1550 of SEQ ID NO:1, or the complementary strand thereto, or the cDNA contained in the deposited clone. In this context “about” includes the particularly recited ranges, larger or smaller by several (5, 4, 3, 2, or 1) nucleotides, at either terminus or at both termini.

In specific embodiments, the polynucleotide fragments of the invention comprise, or alternatively, consist of, a sequence from nucleotides 96 to 374, 453 to 740, and/or 741 to 1094 of SEQ ID NO:1, or the complementary strand thereto. In additional specific embodiments, the polynucleotide fragments of the invention comprise, or alternatively, consist of, a sequence from nucleotides 165 to 221, 291 to 329, 528 to 581, 657 to 695, 816 to 872, and/or 1008 to 1046 of SEQ ID NO:1, or the complementary strand thereto. In further specific embodiments, the polynucleotide fragments of the invention comprise, or alternatively, consist of, a sequence from nucleotides 174 to 212, 300 to 320, 537 to 575, 666 to 686, 825 to 863, and/or 1017 to 1037 of SEQ ID NO:1, or the complementary strand thereto.

Preferably, the polynucleotide fragments of the invention encode a polypeptide which demonstrates a FGFR5 functional activity. By a polypeptide demonstrating “functional activity” is meant, a polypeptide capable of displaying one or more known functional activities associated with members of the FGF receptor family. Such functional activities include, but are not limited to, biological activity (e.g., ability to bind members of the FGF family), antigenicity (ability to bind (or compete with a FGFR5 polypeptide for binding) to an anti-FGFR5 antibody), immunogenicity (ability to generate antibody which binds to a FGFR5 polypeptide), and ability to bind to a receptor or ligand for a FGFR5 polypeptide (e.g., members of the FGF family)).

Preferred nucleic acid fragments of the invention include nucleic acid molecules encoding one or more FGFR5 receptor domains. In particular embodiments, such nucleic acid fragments comprise, or alternatively consist of, nucleic acid molecules encoding: a polypeptide selected from the group consisting of: (a) the leader sequence of FGFR5 (amino acid residues Met-1 to Ala-24 of FIGS. 1A-C (SEQ ID NO:2)); (b) the extracellular immunoglobulin domain I of FGFR5 (amino acid residues from about Ala-25 to about Leu-117 of FIGS. 1A-C (SEQ ID NO:2)); (c) the acid box domain of FGFR5 (amino acid residues from about Asp-118 to about Gln-143 of FIGS. 1A-C (SEQ ID NO:2)); (d) the extracellular immunoglobulin domain II of FGFR5 (amino acid residues from about Trp-144 to about Ile-239 of FIGS. 1A-C (SEQ ID NO:2)); (e) the extracellular immunoglobulin domain III of FGFR5 (amino acid residues from about Gln-240 to about Pro-357 of FIGS. 1A-C (SEQ ID NO:2)); (f) the extracellular immunoglobulin domain III of FGFR5 (amino acid residues from about Gln-240 to about Pro-357 of FIGS. 1A-C (SEQ ID NO:2)); (g) the membrane proximal domain of FGFR5 (amino acid residues from about Asp-358 to about Thr-373 of FIGS. 1A-C (SEQ ID NO:2)); (h) the transmembrane domain of FGFR5 (amino acid residues from about Ser-374 to about Lys-403 of FIGS. 1A-C (SEQ ID NO:2)); (i) and the intracellular (or juxtamembrane) domain of FGFR5 (amino acid residues from about Lys-404 to about Cys-504 of FIGS. 1A-C (SEQ ID NO:2)); or (j) any combination of polypeptides (a)-(i) and (k) and a nucleic acid molecule complementary to the sense strand encoding any of polypeptides (a)-(i).

The amino acid residues constituting the extracellular, transmembrane and intracellular domains have been predicted by computer analysis. Thus, as one of ordinary skill would appreciate, the amino acid residues constituting these domains may vary slightly (e.g., by about 1 to about 15 amino acid residues) depending on the criteria used to define each domain.

Preferred nucleic acid fragments of the invention also include nucleic acid molecules encoding epitope-bearing portions of the FGFR5 receptor. In particular, such nucleic acid fragments of the present invention include nucleic acid molecules encoding: a polypeptide comprising, or alternatively consisting of, amino acid residues: from Ala-23 to Pro-37; from Gln-39 to Arg-48; from Cys-51 to Pro-59; from Met-62 to Arg-76; from Pro-81 to Tyr-97; from Ala-101 to Gly-104; from Asp-119 to Leu-170; from Gly-176 to Trp-204; from Lys-209 to Gly-228; from Val-238 to Val-247; from Asp-259 to Gly-262; from Cys-268 to Lys-275; from Lys-282 to Lys-302; from Pro-307 to Leu-320; from Thr-326 to Gly-334; from Leu-356 to Leu-375; from Ala-401 to Leu-435; from Ala-440 to Gly-443; from Leu-446 to Ala-455; from Pro-462 to Tyr-475; and from His-483 to Val-496; of SEQ ID NO:2. The inventors have determined that the above polypeptides are antigenic regions of the FGFR5 polypeptide. Methods for determining other such epitope-bearing portions of FGFR5 polypeptides are described in detail below.

In another embodiment, the invention provides isolated nucleic acid molecules comprising polynucleotides which hybridize, preferably under stringent hybridization conditions, to a portion of one or more of the nucleic acids (i.e., polynucleotides) described herein, such as, for instance, the cDNA clone contained in ATCC Deposit 203382, the polynucleotide sequence depicted in FIGS. 1A-C (SEQ ID NO:1) or the complementary strand thereto, and/or any of the polynucleotide fragments as described herein. By “stringent hybridization conditions” is intended overnight incubation at 42° C. in a solution comprising: 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65° C. By a polynucleotide which hybridizes to a “portion” of a polynucleotide is intended a polynucleotide (either DNA or RNA) hybridizing to at least 15 nucleotides (nt), and more preferably at least 20 nt, still more preferably at least 30 nt, and even more preferably 30-70, or 80-150 nt, or the entire length of the reference polynucleotide. By a portion of a polynucleotide of “at least 20 nt in length,” for example, is intended 20 or more contiguous nucleotides from the nucleotide sequence of the reference polynucleotide (e.g., the deposited cDNA or the complementary strand of the nucleotide sequence shown in FIGS. 1A-C (SEQ ID NO:1)). Of course, a polynucleotide which hybridizes only to a poly A sequence (such as the 3′ terminal poly(A) tail of a cDNA sequence), or to a complementary stretch of T (or U) residues, would not be included in a polynucleotide of the invention used to hybridize to a portion of a nucleic acid of the invention, since such a polynucleotide would hybridize to any nucleic acid molecule containing a poly (A) stretch or the complement thereof (i.e., practically any double-stranded cDNA clone generated using oligo dT as a primer). These polynucleotides have uses which include, but are not limited to, diagnostic probes and primers as discussed above and in more detail below.

In specific embodiments, the nucleic acid molecules of the invention hybridize to the complementary strand of nucleotides 174 to 212, 300 to 320, 537 to 575, 666 to 686, 825 to 863, and/or 1017 to 1037 of SEQ ID NO:1.

As indicated, nucleic acid molecules of the present invention which encode FGFR5 polypeptides may include, but are not limited to, those encoding the amino acid sequences of the full-length polypeptide (SEQ ID NO:2), by itself; the coding sequence for full-length polypeptide together with additional, non-coding sequences, including for example, but not limited to, introns and non-coding 5′ and 3′ sequences, such as the transcribed, non-translated sequences that play a role in transcription, mRNA processing, including splicing and polyadenylation signals, for example— ribosome binding and stability of mRNA; an additional coding sequence which codes for additional amino acids, such as those which provide additional functionalities. Thus, the sequence encoding the polypeptides may be fused to a marker sequence, such as a sequence encoding a peptide which facilitates purification of the fused polypeptide. In certain preferred embodiments, the marker amino acid sequence is a hexa-histidine peptide, such as the tag provided in a pQE vector (Qiagen, Inc.), among others, many of which are commercially available. As described in Gentz et al., Proc. Natl. Acad. Sci. USA 86:821-824 (1989), for instance, hexa-histidine provides for convenient purification of the fusion protein. The “HA” tag is another peptide useful for purification which corresponds to an epitope derived from the influenza hemagglutinin protein, which has been described by Wilson et al., Cell 37:767 (1984). As discussed below, other such fusion proteins include the FGFR5 receptors fused to IgG-Fc at the N- or C-terminus.

The present invention further relates to variants of the nucleic acid molecules of the present invention, which encode fragments (i.e., portions), analogs or derivatives of the FGFR5 receptor. Variants may occur naturally, such as a natural allelic variant. By an “allelic variant” is intended one of several alternate forms of a gene occupying a given locus on a chromosome of an organism. Genes II, Lewin, B., ed., John Wiley & Sons, New York (1985). Non-naturally occurring variants may be produced using art-known mutagenesis techniques.

Such variants include those produced by nucleotide substitutions, deletions or additions, which may involve one or more nucleotides. The variants may be altered in coding regions, non-coding regions, or both. Alterations in the coding regions may produce conservative or non-conservative amino acid substitutions, deletions or additions. Especially preferred among these are silent substitutions, additions and deletions, which do not alter the properties and activities of the FGFR5 receptor or fragments thereof. Also especially preferred in this regard are conservative substitutions.

Further embodiments of the invention include isolated nucleic acid molecules comprising a polynucleotide having a nucleotide sequence at least 90% identical, and more preferably at least 95%, 96%, 97%, 98% or 99% identical to: (a) a nucleotide sequence encoding the FGFR5 polypeptide amino acid sequence shown in FIGS. 1A-C (SEQ ID NO:2); (b) a nucleotide sequence encoding the FGFR5 polypeptide having the amino acid sequence encoded by the cDNA clone contained in ATCC Deposit Number 203382; (c) a nucleotide sequence encoding the leader sequence of FGFR5 (amino acid residues Met-1 to Ala-24 of FIGS. 1A-C (SEQ ID NO:2)); (d) a nucleotide sequence encoding the extracellular immunoglobulin domain I of FGFR5 (amino acid residues from about Ala-25 to about Leu-117 of FIGS. 1A-C (SEQ ID NO:2)); (e) a nucleotide sequence encoding the acid box domain of FGFR5 (amino acid residues from about Asp-118 to about Gln-143 of FIGS. 1A-C (SEQ ID NO:2)); (f) a nucleotide sequence encoding the extracellular immunoglobulin domain II of FGFR5 (amino acid residues from about Trp-144 to about Ile-239 of FIGS. 1A-C (SEQ ID NO:2)); (g) a nucleotide sequence encoding the extracellular immunoglobulin domain III of FGFR5 (amino acid residues from about Gln-240 to about Pro-357 of FIGS. 1A-C (SEQ ID NO:2)); (h) a nucleotide sequence encoding the extracellular immunoglobulin domain III of FGFR5 (amino acid residues from about Gln-240 to about Pro-357 of FIGS. 1A-C (SEQ ID NO:2)); (i) a nucleotide sequence encoding the membrane proximal domain of FGFR5 (amino acid residues from about Asp-358 to about Thr-373 of FIGS. 1A-C (SEQ ID NO:2)); (j) a nucleotide sequence encoding the transmembrane domain of FGFR5 (amino acid residues from about Ser-374 to about Lys-403 of FIGS. 1A-C (SEQ ID NO:2)); (k) and a nucleotide sequence encoding the intracellular (or juxtamembrane) domain of FGFR5 (amino acid residues from about Lys-404 to about Cys-504 of FIGS. 1A-C (SEQ ID NO:2)); (1) any polynucleotide encoding the polypeptide fragments described herein; (m) the polynucleotide encoding the polypeptide sequence of FIGS. 1A-C (SEQ ID NO:2) minus the leader sequence; minus one, two or three of the extracellular domains; minus the acid box domain; minus the membrane proximal domain; minus the transmembrane domain; and/or minus the intracellular domain of the FGFR5 receptor shown in FIGS. 1A-C (SEQ ID NO:2); and (n) a nucleotide sequence complementary to the nucleotide sequence in (a), (b), (c), (d), (e), (f), (g), (h), (i), (j), (k), (1), or (m).

However, many polynucleotide sequences, such as EST sequences, are publicly available and accessible through sequence databases. Some of these sequences are related to SEQ ID NO:1 and may have been publicly available prior to conception of the present invention. Preferably, such related polynucleotides are specifically excluded from the scope of the present invention. Accordingly, preferably excluded from the present invention are one or more polynucleotides comprising a nucleotide sequence described by the general formula of a-b, where a is any integer between 1 to 3112 of SEQ ID NO:1, b is an integer of 15 to 3097, where both a and b correspond to the positions of nucleotide residues shown in SEQ ID NO:1, and where the b is greater than or equal to a+14.

In another embodiment, the polynucleotides of SEQ ID NO:13 (Genbank Accession No. AI245701) and the amino acid sequence(s) encoded by SEQ ID NO:13, as well as any fragments (as defined herein) thereof, are also excluded from the scope of the present invention.

By a polynucleotide having a nucleotide sequence at least, for example, 95% “identical” to a reference nucleotide sequence encoding a FGFR5 polypeptide is intended that the nucleotide sequence of the polynucleotide is identical to the reference sequence except that the polynucleotide sequence may include up to five point mutations per each 100 nucleotides of the reference nucleotide sequence encoding the FGFR5 receptor. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. The reference (query) sequence may be the FGFR5 encoding nucleotide sequence shown in FIGS. 1A-C (SEQ ID NO:1) or any FGFR5 polynucleotide fragment as described herein.

As a practical matter, whether any particular nucleic acid molecule is at least 90%, 95%, 96%, 97%, 98% or 99% identical to, for instance, the encoding nucleotide sequence shown in FIGS. 1A-C (SEQ ID NO:1), or to the nucleotide sequence of the deposited cDNA clone, can be determined conventionally using known computer programs such as the MegAlign program, which is included in the suite of computer applications contained within the DNASTAR program. When using MegAlign or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

In a specific embodiment, the identity between a reference (query) sequence (a sequence of the present invention) and a subject sequence, also referred to as a global sequence alignment, is determined using the FASTDB computer program based on the algorithm of Brutlag et al. (Comp. App. Biosci. 6:237-245 (1990)). Preferred parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the length of the subject nucleotide sequence, whichever is shorter. According to this embodiment, if the subject sequence is shorter than the query sequence because of 5′ or 3′ deletions, not because of internal deletions, a manual correction is made to the results to take into consideration the fact that the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. A determination of whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of this embodiment. Only bases outside the 5′ and 3′ bases of the subject sequence, as displayed by the FASTDB alignment, which are not matched/aligned with the query sequence, are calculated for the purposes of manually adjusting the percent identity score. For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for. No other manual corrections are made for the purposes of this embodiment.

The present application is directed to nucleic acid molecules at least 90%, 95%, 96%, 97%, 98% or 99% identical to the nucleic acid sequences (i.e., polynucleotides) disclosed herein, irrespective of whether they encode a polypeptide having FGFR5 functional activity. This is because even where a particular nucleic acid molecule does not encode a polypeptide having FGFR5 functional activity, one of skill in the art would still know how to use the nucleic acid molecule, for instance, as a hybridization probe or a polymerase chain reaction (PCR) primer. Uses of the nucleic acid molecules of the present invention that do not encode a polypeptide having FGFR5 functional activity include, but are not limited to, inter alia, (1) isolating a FGFR5 receptor gene or allelic or splice variants thereof in a cDNA library; (2) in situ hybridization (e.g., “FISH”) to metaphase chromosomal spreads to provide precise chromosomal location of a FGFR5 receptor gene, as described in Verma et al., Human Chromosomes: A Manual of Basic Techniques, Pergamon Press, New York (1988); and (3) Northern Blot analysis for detecting FGFR5 mRNA expression in specific tissues.

Preferred, however, are nucleic acid molecules having sequences at least 90%, 95%, 96%, 97%, 98% or 99% identical to the nucleic acid sequences disclosed herein, which do, in fact, encode a polypeptide having FGFR5 functional activity. By “a polypeptide having FGFR5 receptor functional activity” is intended polypeptides exhibiting activity similar, but not necessarily identical, to an activity of the FGFR5 receptors of the present invention (either the full-length polypeptide, or the splice variants), as measured, for example, in a particular immunoassay or biological assay. For example, FGFR5 activity can be measured by determining the ability of a FGFR5 polypeptide to bind a FGFR5 ligand (e.g., members of the FGF family) and/or to mediate the activities of members of the fibroblast growth factor family. FGFR5 receptor activity may also be measured by determining the ability of a polypeptide, such as a cognate ligand which is free or expressed on a cell surface, to induce cation flux in cells expressing the polypeptide.

Of course, due to the degeneracy of the genetic code, one of ordinary skill in the art will immediately recognize that a large number of the nucleic acid molecules having a sequence at least 90%, 95%, 96%, 97%, 98%, or 99% identical to the nucleic acid sequence of the deposited cDNA, the nucleic acid sequence shown in FIGS. 1A-C (SEQ ID NO:1), or fragments thereof, will encode polypeptides “having FGFR5 functional activity.” In fact, since degenerate variants of any of these nucleotide sequences all encode the same polypeptide, in many instances, this will be clear to the skilled artisan even without performing the above described comparison assay. It will be further recognized in the art that, for such nucleic acid molecules that are not degenerate variants, a reasonable number will also encode a polypeptide having FGFR5 functional activity. This is because the skilled artisan is fully aware of amino acid substitutions that are either less likely or not likely to significantly effect protein function (e.g., replacing one aliphatic amino acid with a second aliphatic amino acid).

For example, guidance concerning how to make phenotypically silent amino acid substitutions is provided in Bowie et al., “Deciphering the Message in Protein Sequences: Tolerance to Amino Acid Substitutions,” Science 247:1306-1310 (1990), wherein the authors indicate that proteins are surprisingly tolerant of amino acid substitutions.

### Vectors and Host Cells

The present invention also relates to vectors which include the isolated DNA molecules (i.e., polynucleotides) of the present invention, host cells which are genetically engineered with the recombinant vectors, and the production of FGFR5 polypeptides or fragments thereof using these host cells or host cells that have otherwise been genetically engineered using techniques known in the art to express a polypeptide of the invention.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

In one embodiment, the polynucleotide of the invention is operatively associated with an appropriate heterologous regulatory element (e.g., promoter or enhancer), such as the phage lambda PL promoter, the E. coli lac, trp and tac promoters, the SV40 early and late promoters and promoters of retroviral LTRs, to name a few. Other suitable promoters or enhancers will be known to the skilled artisan.

In embodiments in which vectors contain expression constructs, these constructs will further contain sites for transcription initiation, termination and, in the transcribed region, a ribosome binding site for translation. The coding portion of the mature transcripts expressed by the vector expression constructs will preferably include a translation initiating at the beginning and a termination codon (UAA, UGA or UAG) appropriately positioned at the end of the polypeptide to be translated.

As indicated, the expression vectors will preferably include at least one selectable marker. Such markers include dihydrofolate reductase or neomycin resistance for eukaryotic cell culture and tetracycline or ampicillin resistance genes for culturing in E. coli and other bacteria. Representative examples of appropriate heterologous hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS and Bowes melanoma cells; and plant cells. Appropriate culture mediums and conditions for the above-described host cells are known in the art.

Among vectors preferred for use in bacteria include pQE70, pQE60 and pQE-9, available from Qiagen; pHE4, pA2; and P04, pBS vectors, Phagescript vectors, Bluescript vectors, pNH8A, pNH16a, pNH18A, pNH46A, available from Stratagene; and ptrc99a, pKK223-3, pKK233-3, pVR240, pRIT5 available from Pharmacia. Among preferred eukaryotic vectors are pWLNEO, pSV2CAT, pOG44, pXT1 and pSG available from Stratagene; pSVK3, pBPV, pMSG and pSVL available from Pbarmacia. Other suitable vectors will be readily apparent to the skilled artisan.

Selection of appropriate vectors and promoters for expression in a host cell is a well known procedure and the requisite techniques for expression vector construction, introduction of the vector into the host and expression in the host are routine skills in the art.

In addition to encompassing host cells containing the vector constructs discussed herein, the invention also encompasses primary, secondary, and immortalized host cells of vertebrate origin, particularly mammalian origin, that have been engineered to delete or replace endogenous genetic material (e.g., FGFR5 coding sequence), and/or to include genetic material (e.g., heterologous polynucleotide sequences) that is operably associated with FGFR5 polynucleotides of the invention, and which activates, alters, and/or amplifies endogenous FGFR5 polynucleotides. For example, techniques known in the art may be used to operably associate heterologous control regions (e.g., promoter and/or enhancer) and endogenous FGFR5 polynucleotide sequences via homologous recombination (see, e.g., U.S. Pat. No. 5,641,670, issued Jun. 24, 1997; International Publication No. WO 96/29411, published Sep. 26, 1996; International Publication No. WO 94/12650, published Aug. 4, 1994; Koller et al., Proc. Natl. Acad. Sci. USA 86:8932-8935 (1989); and Zijlstra et al., Nature 342:435-438 (1989), the disclosures of each of which are incorporated by reference in their entireties).

The host cell can be a higher eukaryotic cell, such as a mammalian cell (e.g., a human derived cell), or a lower eukaryotic cell, such as a yeast cell, or the host cell can be a prokaryotic cell, such as a bacterial cell. The host strain may be chosen which modulates the expression of the inserted gene sequences, or modifies and processes the gene product in the specific fashion desired. Expression from certain promoters can be elevated in the presence of certain inducers; thus expression of the genetically engineered polypeptide may be controlled. Furthermore, different host cells have characteristics and specific mechanisms for the translational and post-translational processing and modification (e.g., glycosylation, phosphorylation, cleavage) of proteins. Appropriate cell lines can be chosen to ensure the desired modifications and processing of the foreign protein expressed.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986).

The polypeptide may be expressed in a modified form, such as a fusion protein (comprising the polypeptide joined via a peptide bond to a heterologous protein sequence (of a different protein)), and may include not only secretion signals, but also additional heterologous functional regions. Such a fusion protein can be made by ligating polynucleotides of the invention and the desired nucleic acid sequence encoding the desired amino acid sequence to each other, by methods known in the art, in the proper reading frame, and expressing the fusion protein product by methods known in the art. Alternatively, such a fusion protein can be made by protein synthetic techniques, e.g., by use of a peptide synthesizer. Thus, for instance, a region of additional amino acids, particularly charged amino acids, may be added to the N-terminus of the polypeptide to improve stability and persistence in the host cell, during purification, or during subsequent handling and storage. Additionally, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art. A preferred fusion protein comprises a heterologous region from immunoglobulin that is useful to solubilize proteins. For example, EP-A-O 464 533 (Canadian counterpart 2045869) discloses fusion proteins comprising various portions of constant region of immunoglobin molecules together with another human protein or part thereof. In many cases, the Fc part in a fusion protein is thoroughly advantageous for use in therapy and diagnosis and thus results, for example, in improved pharmacokinetic properties (EP-A 0232 262). On the other hand, for some uses it would be desirable to be able to delete the Fc part after the fusion protein has been expressed, detected and purified in the advantageous manner described. This is the case when Fc portion proves to be a hindrance to use in therapy and diagnosis, for example when the fusion protein is to be used as antigen for immunizations. In drug discovery, for example, human proteins, such as, human hIL-5 receptor have been fused with Fc portions for the purpose of high-throughput screening assays to identify antagonists of hIL-5. See, Bennett et al., J. Mol. Recog. 8:52-58 (1995) and Johanson et al., J. Biol. Chem. 270(16):9459-9471 (1995).

FGFR5 polypeptides (including fragments, variants, derivatives, and analogs thereof) can be recovered and purified from recombinant cell cultures by standard methods which include, but are not limited to, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification. Polypeptides of the present invention include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, or alternatively, may be missing the N-terminal methonine, in some cases as a result of host-mediated processes.

In addition, proteins of the invention can be chemically synthesized using techniques known in the art (e.g., see Creighton, 1983, Proteins: Structures and Molecular Principles, W. H. Freeman & Co., N.Y., and Hunkapiller, M., et al., Nature 310:105-111 (1984)). For example, a peptide corresponding to a fragment of the FGFR5 polypeptides of the invention can be synthesized by use of a peptide synthesizer. Furthermore, if desired, nonclassical amino acids or chemical amino acid analogs can be introduced as a substitution or addition into the FGFR5 polypeptide sequence. Non-classical amino acids include, but are not limited to, the D-isomers of the common amino acids, 2,4-diaminobutyric acid, a-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, b-alanine, fluoroamino acids, designer amino acids such as b-methyl amino acids, Ca-methyl amino acids, Na-methyl amino acids, and amino acid analogs in general. Furthermore, the amino acid can be D (dextrorotary) or L (levorotary).

The invention additionally encompasses FGFR5 polypeptides which are differentially modified during or after translation, e.g., by glycosylation, acetylation, phosphorylation, amidation, iodination, derivatization by known protecting/blocking groups, proteolytic cleavage, linkage to an antibody molecule or other cellular ligand, etc. Any of numerous chemical modifications may be carried out by known techniques, including but not limited to, specific chemical cleavage by cyanogen bromide, trypsin, chymotrypsin, papain, V8 protease, NaBH₄, acetylation, formylation, oxidation, reduction, metabolic synthesis in the presence of tunicamycin; etc.

Additional post-translational modifications encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. The polypeptides may also be modified with a detectable label, such as an enzymatic, fluorescent, isotopic or affinity label to allow for detection and isolation of the protein.

Also provided by the invention are chemically modified derivatives of FGFR5 which may provide additional advantages such as increased solubility, stability and circulating time of the polypeptide, or decreased immunogenicity (see U.S. Pat. No. 4,179,337). The chemical moieties for derivitization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

The polyethylene glycol molecules (or other chemical moieties) should be attached to the protein with consideration of effects on functional or antigenic domains of the protein. There are a number of attachment methods available to those skilled in the art, e.g., EP 0 401 384, herein incorporated by reference (coupling PEG to G-CSF), see also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

One may specifically desire proteins chemically modified at the N-terminus. Using polyethylene glycol as an illustration of the present composition, one may select from a variety of polyethylene glycol molecules (by molecular weight, branching, etc.), the proportion of polyethylene glycol molecules to protein (or peptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus modification may be accomplished by reductive alkylation which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

### FGFR5 Polypeptides and Fragments

The invention further provides for the proteins containing polypeptide sequences encoded by the polynucleotides of the invention.

The FGFR5 proteins of the invention may be in monomers or multimers (i.e., dimers, trimers, tetramers, and higher multimers). Accordingly, the present invention relates to monomers and multimers of the FGFR5 proteins of the invention, their preparation, and compositions (preferably, pharmaceutical compositions) containing them. In specific embodiments, the polypeptides of the invention are monomers, dimers, trimers or tetramers. In additional embodiments, the multimers of the invention are at least dimers, at least trimers, or at least tetramers.

Multimers encompassed by the invention may be homomers or heteromers. As used herein, the term homomer, refers to a multimer containing only FGFR5 proteins of the invention (including FGFR5 fragments, variants, and fusion proteins, as described herein). These homomers may contain FGFR5 proteins having identical or different polypeptide sequences. In a specific embodiment, a homomer of the invention is a multimer containing only FGFR5 proteins having an identical polypeptide sequence. In another specific embodiment, a homomer of the invention is a multimer containing FGFR5 proteins having different polypeptide sequences. In specific embodiments, the multimer of the invention is a homodimer (e.g., containing FGFR5 proteins having identical or different polypeptide sequences) or a homotrimer (e.g., containing FGFR5 proteins having identical or different polypeptide sequences). In additional embodiments, the homomeric multimer of the invention is at least a homodimer, at least a homotrimer, or at least a homotetramer.

As used herein, the term heteromer refers to a multimer containing heterologous proteins (i.e., proteins containing only polypeptide sequences that do not correspond to a polypeptide sequences encoded by the FGFR5 gene) in addition to the FGFR5 proteins of the invention. In a specific embodiment, the multimer of the invention is a heterodimer, a heterotrimer, or a heterotetramer. In additional embodiments, the homomeric multimer of the invention is at least a homodimer, at least a homotrimer, or at least a homotetramer.

Multimers of the invention may be the result of hydrophobic, hydrophilic, ionic and/or covalent associations and/or may be indirectly linked, by for example, liposome formation. Thus, in one embodiment, multimers of the invention, such as, for example, homodimers or homotrimers, are formed when proteins of the invention contact one another in solution. In another embodiment, heteromultimers of the invention, such as, for example, heterotrimers or heterotetramers, are formed when proteins of the invention contact antibodies to the polypeptides of the invention (including antibodies to the heterologous polypeptide sequence in a fusion protein of the invention) in solution. In other embodiments, multimers of the invention are formed by covalent associations with and/or between the FGFR5 proteins of the invention. Such covalent associations may involve one or more amino acid residues contained in the polypeptide sequence of the protein ( e.g., the polypeptide sequence recited in SEQ ID NO:2 or the polypeptide encoded by the deposited cDNA clone). In one instance, the covalent associations are cross-linking between cysteine residues located within the polypeptide sequences of the proteins which interact in the native (i.e., naturally occurring) polypeptide. In another instance, the covalent associations are the consequence of chemical or recombinant manipulation. Alternatively, such covalent associations may involve one or more amino acid residues contained in the heterologous polypeptide sequence in a FGFR5 fusion protein. In one example, covalent associations are between the heterologous sequence contained in a fusion protein of the invention (see, e.g., U.S. Pat. No. 5,478,925). In a specific example, the covalent associations are between the heterologous sequence contained in a FGFR5-Fc fusion protein of the invention (as described herein). In another specific example, covalent associations of fusion proteins of the invention are between heterologous polypeptide sequences from another FGFR family ligand/receptor member that is capable of forming covalently associated multimers, such as for example, oseteoprotegerin (see, e.g., International Publication No. WO 98/49305, the contents of which are herein incorporated by reference in its entirety).

The multimers of the invention may be generated using chemical techniques known in the art. For example, proteins desired to be contained in the multimers of the invention may be chemically cross-linked using linker molecules and linker molecule length optimization techniques known in the art (see, e.g., U.S. Pat. No. 5,478,925, which is herein incorporated by reference in its entirety). Additionally, multimers of the invention may be generated using techniques known in the art to form one or more inter-molecule cross-links between the cysteine residues located within the polypeptide sequence of the proteins desired to be contained in the multimer (see, e.g., U.S. Pat. No. 5,478,925, which is herein incorporated by reference in its entirety). Further, proteins of the invention may be routinely modified by the addition of cysteine or biotin to the C terminus or N-terminus of the polypeptide sequence of the protein and techniques known in the art may be applied to generate multimers containing one or more of these modified proteins (see, e.g., U.S. Pat. No. 5,478,925, which is herein incorporated by reference in its entirety). Additionally, techniques known in the art may be applied to generate liposomes containing the protein components desired to be contained in the multimer of the invention (see, e.g., U.S. Pat. No. 5,478,925, which is herein incorporated by reference in its entirety).

Alternatively, multimers of the invention may be generated using genetic engineering techniques known in the art. In one embodiment, proteins contained in multimers of the invention are produced recombinantly using fusion protein technology described herein or otherwise known in the art (see, e.g., U.S. Pat. No. 5,478,925, which is herein incorporated by reference in its entirety). In a specific embodiment, polynucleotides coding for a homodimer of the invention are generated by ligating a polynucleotide sequence encoding a polypeptide of the invention to a sequence encoding a linker polypeptide and then further to a synthetic polynucleotide encoding the translated product of the polypeptide in the reverse orientation from the original C-terminus to the N-terminus (lacking the leader sequence) (see, e.g., U.S. Pat. No. 5,478,925, which is herein incorporated by reference in its entirety). In another embodiment, recombinant techniques described herein or otherwise known in the art are applied to generate recombinant polypeptides of the invention which contain a transmembrane domain and which can be incorporated by membrane reconstitution techniques into liposomes (see, e.g., U.S. Pat. No. 5,478,925, which is herein incorporated by reference in its entirety).

The invention further provides isolated FGFR5 polypeptides having the amino acid sequence encoded by the deposited cDNA (i.e., clone HHERA91), the amino acid sequence depicted in FIGS. 1A-C (SEQ ID NO:2), or a polypeptide comprising a fragment (i.e., portion) of the above polypeptides.

The polypeptides of the invention may be membrane bound or may be in a soluble circulating form. Soluble peptides are defined by amino acid sequence wherein the sequence comprises the polypeptide sequence lacking transmembrane domains.

The polypeptides of the present invention may exist as a membrane bound receptor having a transmembrane region and an intra- and extracellular region or they may exist in soluble form wherein a transmembrane domain is lacking. One example of such a form of the FGFR5 receptor is the FGFR5 polypeptide shown in FIGS. 1A-C (SEQ ID NO:2) which contains, a transmembrane, an intracellular and extracellular domains. Thus, this form of the FGFR5 polypeptide appears to be integrated in the plasma membrane of cells which express this polypeptide.

The polypeptides of the present invention are preferably provided in an isolated form. By “isolated polypeptide”, is intended a polypeptide removed from its native environment. Thus, a polypeptide produced and contained within a recombinant host cell would be considered “isolated” for purposes of the present invention. Also intended as an “isolated polypeptide” are polypeptides that have been purified, partially or substantially, from a recombinant host. For example, recombinantly produced versions of the FGFR5 polypeptides can be substantially purified by the one-step method described in Smith and Johnson, Gene 67:31-40 (1988).

Polypeptide fragments of the present invention include polypeptides comprising or alternatively, consisting of, an amino acid sequence contained in SEQ ID NO:2, encoded by the cDNA contained in the deposited clone, or encoded by nucleic acids which hybridize (e.g., under stringent hybridization conditions) to the nucleotide sequence contained in the deposited clone, or shown in FIGS. 1A-C (SEQ ID NO:1) or the complementary strand thereto (e.g., a polynucleotide encoding a FGFR5 polypeptide fragment described herein). Protein fragments may be “free-standing,” or comprised within a larger polypeptide of which the fragment forms a part or region, most preferably as a single continuous region. Representative examples of polypeptide fragments of the invention, include, for example, fragments that comprise or alternatively, consist of from about amino acid residues: 1 to 50, 51 to 100, 101 to 150, 151 to 172, 173 to 200, 201 to 250, 251 to 300, 301 to 350, 351 to 400, 401 to 450, and/or 451 to 504 of SEQ ID NO:2. Moreover, polypeptide fragments can be at least 10, 20, 30, 40, 50, 60, 70, 80, 90, 100, 110, 120, 130, 140, 150, 175 or 200 amino acids in length

In additional embodiments, the polypeptide fragments of the invention comprise, or alternatively consist, of one or more FGFR5 receptor domains. In particular embodiments, such polypeptide fragments comprise, or alternatively, consist of: (a) a nucleotide sequence encoding the leader sequence of FGFR5 (amino acid residues Met-1 to Ala-24 of FIGS. 1A-C (SEQ ID NO:2)); (b) a nucleotide sequence encoding the extracellular immunoglobulin domain I of FGFR5 (amino acid residues from about Ala-25 to about Leu-117 of FIGS. 1A-C (SEQ ID NO:2)); (c) a nucleotide sequence encoding the acid box domain of FGFR5 (amino acid residues from about Asp-118 to about Gln-143 of FIGS. 1A-C (SEQ ID NO:2)); (d) a nucleotide sequence encoding the extracellular immunoglobulin domain II of FGFR5 (amino acid residues from about Trp-144 to about Ile-239 of FIGS. 1A-C (SEQ ID NO:2)); (e) a nucleotide sequence encoding the extracellular immunoglobulin domain III of FGFR5 (amino acid residues from about Gln-240 to about Pro-357 of FIGS. 1A-C (SEQ ID NO:2)); (f) a nucleotide sequence encoding the extracellular immunoglobulin domain III of FGFR5 (amino acid residues from about Gln-240 to about Pro-357 of FIGS. 1A-C (SEQ ID NO:2)); (g) a nucleotide sequence encoding the membrane proximal domain of FGFR5 (amino acid residues from about Asp-358 to about Thr-373 of FIGS. 1A-C (SEQ ID NO:2)); (h) a nucleotide sequence encoding the transmembrane domain of FGFR5 (amino acid residues from about Ser-374 to about Lys-403 of FIGS. 1A-C (SEQ ID NO:2)); (i) and a nucleotide sequence encoding the intracellular (or juxtamembrane) domain of FGFR5 (amino acid residues from about Lys-404 to about Cys-504 of FIGS. 1A-C (SEQ ID NO:2)); or (j) any combination of polypeptides (a)-(i). Further embodiments of the invention include isolated amino acid molecules comprising a polypeptide having an amino acid sequence at least 80% identical, more preferably at least 85%, 90%, or 95% identical, and still more preferably at least 96%, 97%, 98%, or 99% identical to any of above mentioned polypeptides.

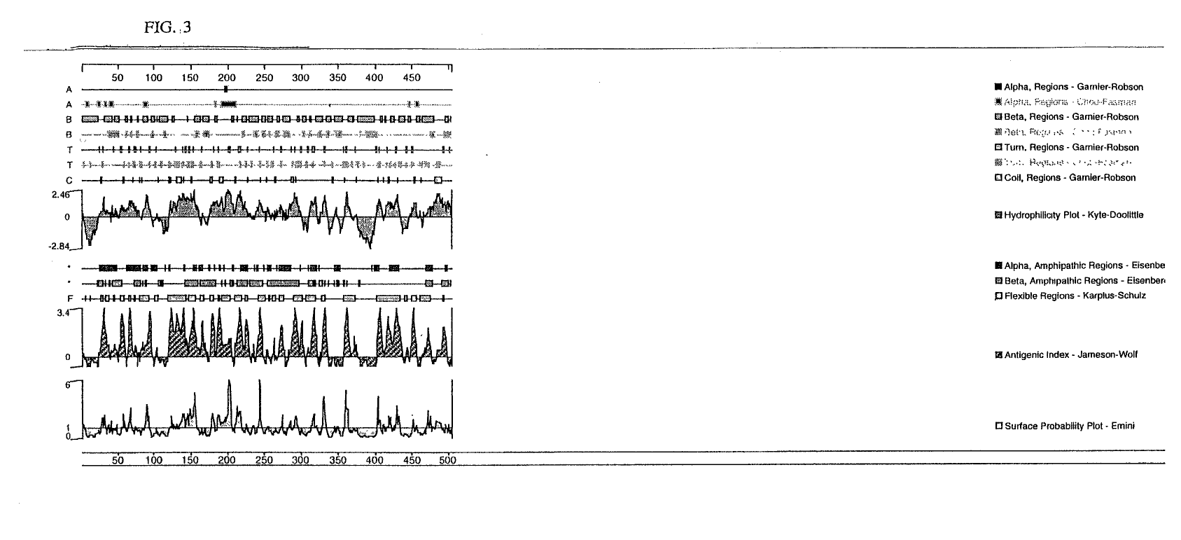

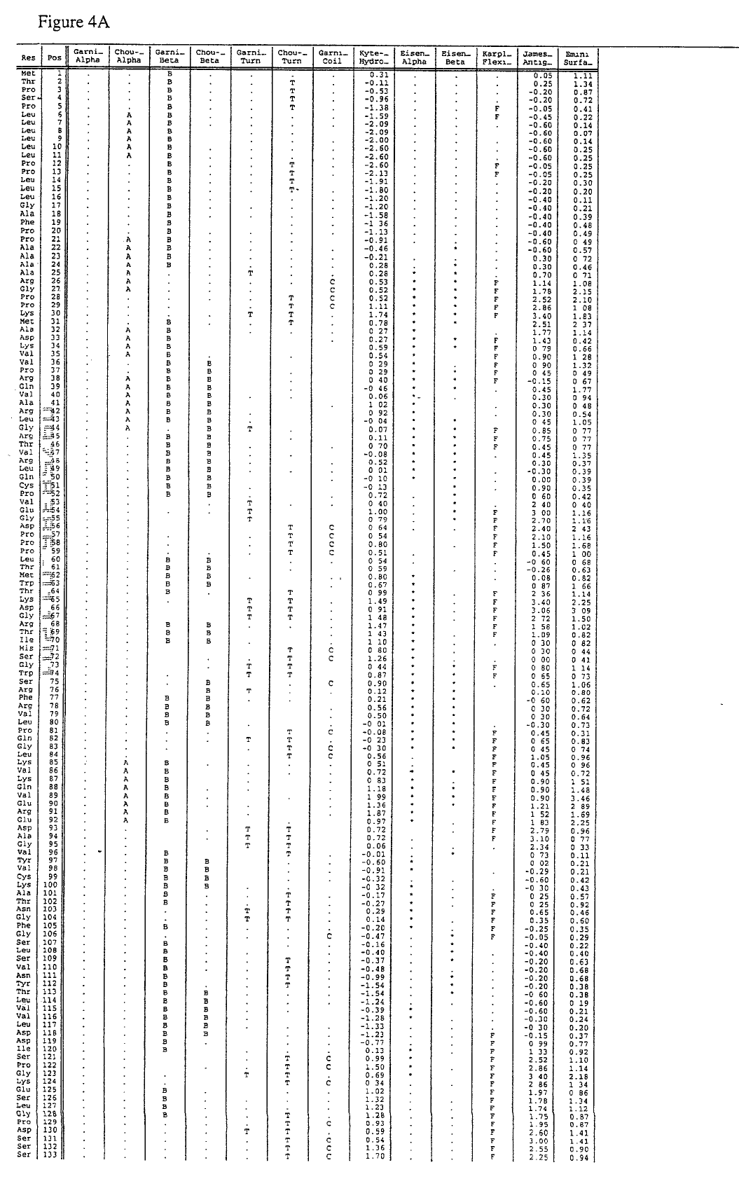

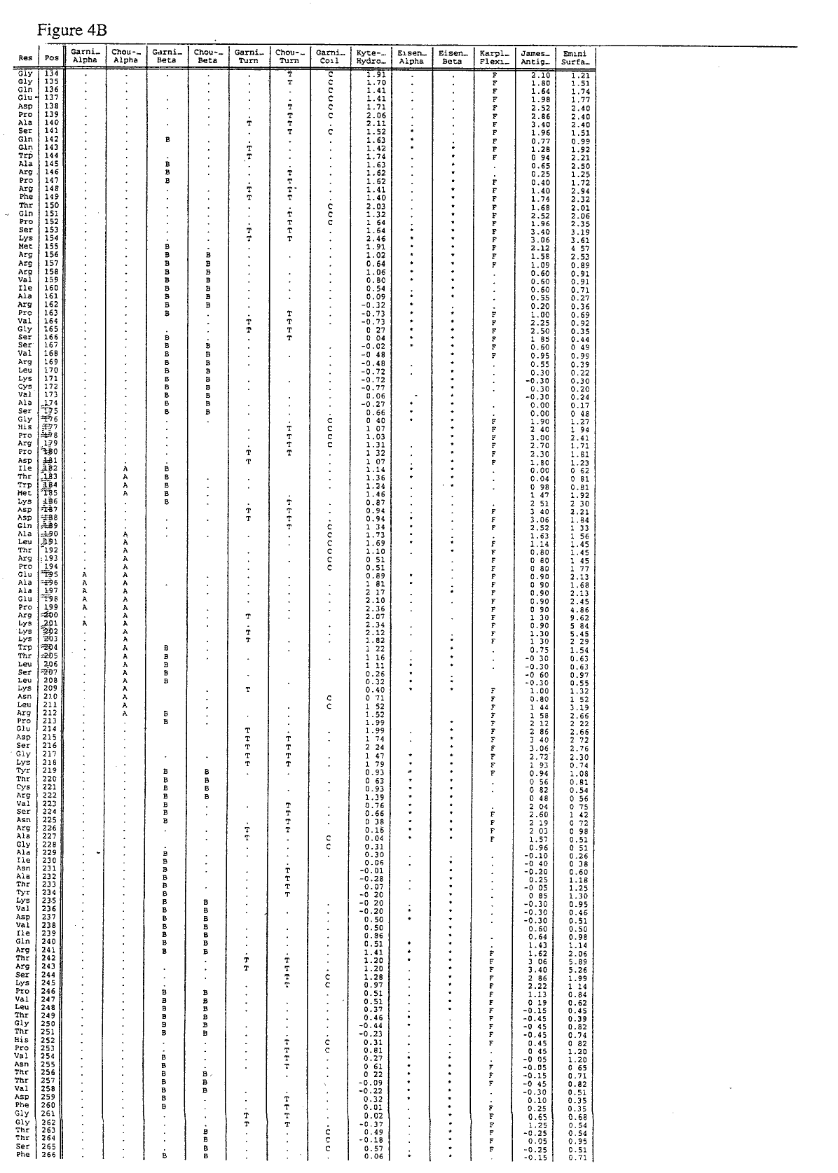

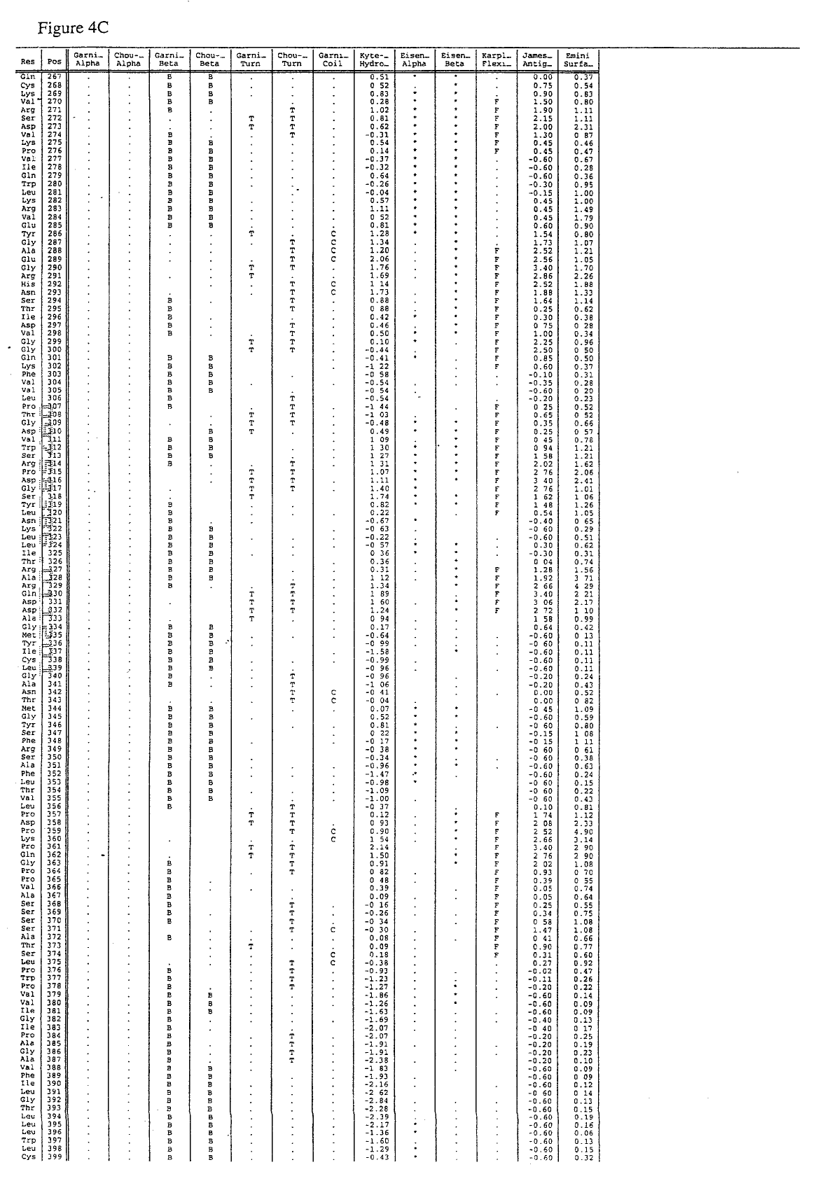

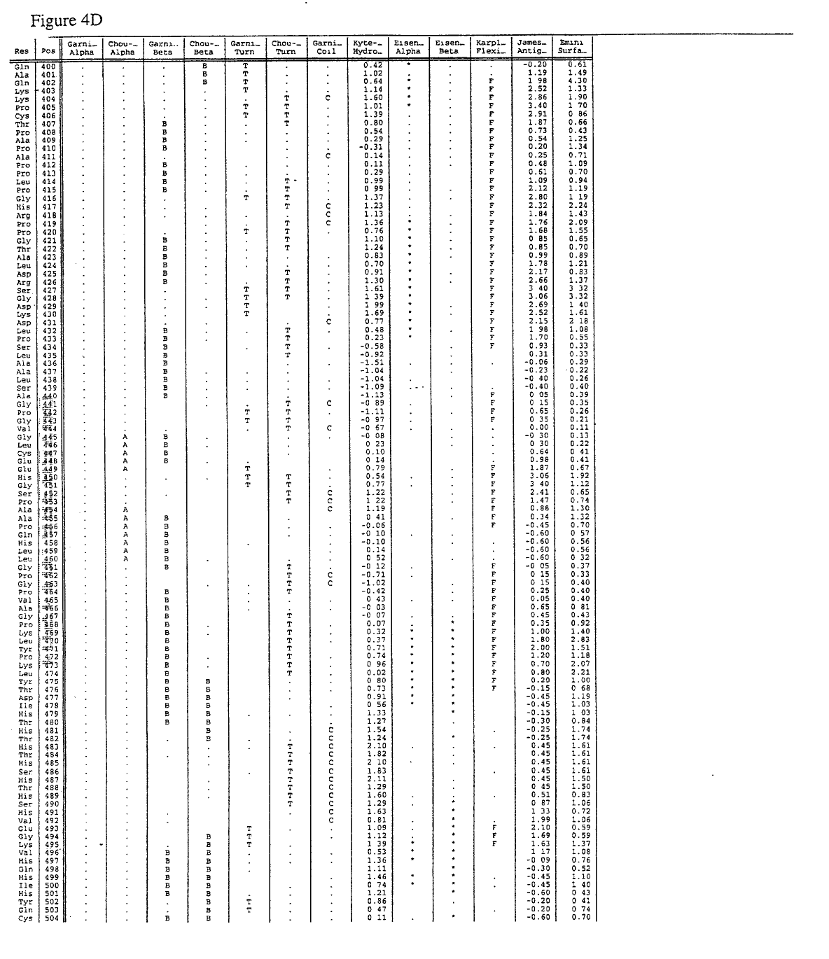

Among the especially preferred fragments of the invention are fragments characterized by structural or functional attributes of FGFR5. Such fragments include amino acid residues that comprise alpha-helix and alpha-helix forming regions (“alpha-regions”), beta-sheet and beta-sheet-forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, surface forming regions, and high antigenic index regions (i.e., containing four or more contiguous amino acids having an antigenic index of greater than or equal to 1.5, as identified using the default parameters of the Jameson-Wolf program) of FGFR5 (SEQ ID NO:2). Certain preferred regions are those set out in FIG. 3 and include, but are not limited to, regions of the aforementioned types identified by analysis of the amino acid sequence depicted in FIGS. 1A-C (SEQ ID NO:2), such preferred regions include; Garnier-Robson predicted alpha-regions, beta-regions, turn-regions, and coil-regions; Chou-Fasman predicted alpha-regions, beta-regions, turn-regions, and coil-regions; Kyte-Doolittle predicted hydrophilic and hydrophobic regions; Eisenberg alpha and beta amphipathic regions; Emini surface-forming regions; and Jameson-Wolf high antigenic index regions, as predicted using the default parameters of these computer programs. Polynucleotides encoding these polypeptides are also encompassed by the invention.

In specific embodiments, polypeptide fragments of the invention comprise, or alternatively consist of, amino acid residues: 48 to 66, 90 to 102, 169 to 186, 212 to 224, 265 to 283, and/or 329 to 341, as depicted in FIGS. 1A-C (SEQ ID NO:2). In further specific embodiments, polypeptide fragments of the invention comprise, or alternatively, consist of, amino acid residues: 51 to 63, 93 to 99, 172 to 184, 215 to 221, 268 to 280, and/or 332 to 338, as depicted in FIGS. 1A-C (SEQ ID NO:2). Polynucleotides encoding these polypeptides are also encompassed by the invention.

The invention also provides polypeptides comprising epitope-bearing portions of the polypeptides of the invention. The epitopes of these polypeptide portions are an immunogenic or antigenic epitopes of the polypeptides described herein. An “immunogenic epitope” is defined as a part of a protein that elicits an antibody response when the whole protein is the immunogen. On the other hand, a region of a protein molecule to which an antibody can bind is defined as an “antigenic epitope.” The number of immunogenic epitopes of a polypeptide generally is less than the number of antigenic epitopes. See, for instance, Geysen et al., Proc. Natl. Acad. Sci. USA 81:3998-4002 (1983).

As to the selection of polypeptides bearing an antigenic epitope (i.e., that contain a region of a polypeptide to which an antibody can bind), it is well known in that art that relatively short synthetic polypeptides that mimic part of a polypeptide sequence are routinely capable of eliciting an antiserum that reacts with the partially mimicked polypeptide. See, for instance, Sutcliffe et al., Science 219:660-666 (1983). Polypeptides capable of eliciting protein-reactive sera are frequently represented in the primary sequence of a polypeptide, can be characterized by a set of simple chemical rules, and are confined neither to immunodominant regions of intact polypeptides (i.e., immunogenic epitopes) nor to the amino or carboxyl terminals.

Antigenic epitope-bearing polypeptides of the invention are therefore useful to raise antibodies, including monoclonal antibodies, that bind specifically to a polypeptide of the invention. See, for instance, Wilson et al., Cell 37:767-778 (1984) at 777. Antigenic epitope-bearing peptides and polypeptides of the invention preferably contain a sequence of at least four, at least seven, more preferably at least nine, and most preferably between at least about 15 to about 30 amino acids contained within the amino acid sequence of a polypeptide of the invention.

Non-limiting examples of antigenic polypeptides that can be used to generate FGFR5 receptor-specific antibodies include: a polypeptide comprising amino acid residues from: Ala-23 to Pro-37; from Gln-39 to Arg-48; from Cys-51 to Pro-59; from Met-62 to Arg-76; from Pro-81 to Tyr-97; from Ala-101 to Gly-104; from Asp-119 to Leu-170; from Gly-176 to Trp-204; from Lys-209 to Gly-228; from Val-238 to Val-247; from Asp-259 to Gly-262; from Cys-268 to Lys-275; from Lys-282 to Lys-302; from Pro-307 to Leu-320; from Thr-326 to Gly-334; from Leu-356 to Leu-375; from Ala-401 to Leu-435; from Ala-440 to Gly-443; from Leu-446 to Ala-455; from Pro-462 to Tyr-475; and from His-483 to Val-496 as depicted in FIGS. 1A-C (SEQ ID NO:2). In further preferred embodiments, polypeptide fragments of the invention compose 2, 3, 4, 5, 6, 7, 8, 9, 10, 15, or all 21 of the above recited FGFR5 antigenic regions. As indicated above, the inventors have determined that the above polypeptide fragments are antigenic regions of the FGFR5 polypeptide.

The epitope-bearing peptides and polypeptides of the invention may be produced by any conventional means. Houghten, R. A. (1985) General method for the rapid solid-phase synthesis of large numbers of peptides: specificity of antigen-antibody interaction at the level of individual amino acids. Proc. Natl. Acad. Sci. USA 82:5131-5135. This “Simultaneous Multiple Peptide Synthesis (SMPS)” process is further described in U.S. Pat. No. 4,631,211 to Houghten et al. (1986).

As one of skill in the art will appreciate, FGFR5 polypeptides of the present invention and the epitope-bearing fragments thereof described above can be combined with parts of the constant domain of immunoglobulins (IgG), resulting in chimeric polypeptides. These fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EPA 394,827; Traunecker et al., Nature 331:84-86 (1988)). Fusion proteins that have a disulfide-linked dimeric structure due to the IgG part can also be more efficient in binding and neutralizing other molecules than the monomeric FGFR5 receptor polypeptides or polypeptide fragments alone (Fountoulakis et al., J. Biochem 270:3958-3964 (1995)).

For many proteins, including the extracellular domain of a membrane associated protein, it is known in the art that one or more amino acids may be deleted from the N-terminus or C-terminus without substantial loss of biological function. However, even if deletion of one or more amino acids from the N-terminus or C-terminus of a protein results in modification or loss of one or more biological functions of the protein, other FGFR5 functional activities may still be retained. For example, in many instances, the ability of the shortened protein to induce and/or bind to antibodies which recognize FGFR5 (preferably antibodies that bind specifically to FGFR5) will be retained irrespective of the size or location of the deletion. Whether a particular polypeptide lacking N-terminal and/or C-terminal residues of a complete protein retains such immunologic activities can readily be determined by routine methods described herein and otherwise known in the art.

In one embodiment, the present invention further provides polypeptides having one or more residues deleted from the amino terminus of the amino acid sequence of the FGFR5 polypeptide depicted in FIGS. 1A-C (SEQ ID NO:2) or encoded by the cDNA of the deposited clone. Particularly, in one embodiment, N-terminal deletions of the FGFR5 polypeptide can be described by the general formula m to 504, where m is an integer from 2 to 504 corresponding to the position of amino acid identified in SEQ ID NO:2 and preferably, corresponds to one of the N-terminal amino acid residues identified in the N-terminal deletions specified herein. In specific embodiments, N-terminal deletions of the FGFR5 polypeptide of the invention comprise, or alternatively, consist of, amino acid residues: T-2 to C-504; P-3 to C-504; S-4 to C-504; P-5 to C-504; L-6 to C-504; L-7 to C-504; L-8 to C-504; L-9 to C-504; L-10 to C-504; L-11 to C-504; P-12 to C-504; P-13 to C-504; L-14 to C-504; L-15 to C-504; L-16 to C-504; G-17 to C-504; A-18 to C-504; F-19 to C-504; P-20 to C-504; P-21 to C-504; A-22 to C-504; A-23 to C-504; A-24 to C-504; A-25 to C-504; R-26 to C-504; G-27 to C-504; P-28 to C-504; P-29 to C-504; K-30 to C-504; M-31 to C-504; A-32 to C-504; D-33 to C-504; K-34 to C-504; V-35 to C-504; V-36 to C-504; A-37 to C-504; R-38 to C-504; Q-39 to C-504; V-40 to C-504; A-41 to C-504; R-42 to C-504; L-43 to C-504; G-44 to C-504; R-45 to C-504; T-46 to C-504; V-47 to C-504; R-48 to C-504; L-49 to C-504; Q-50 to C-504; C-51 to C-504; P-52 to C-504; V-53 to C-504; E-54 to C-504; G-55 to C-504; D-56 to C-504; P-57 to C-504; P-58 to C-504; P-59 to C-504; L-60 to C-504; T-61 to C-504; M-62 to C-504; W-63 to C-504; T-64 to C-504; K-65 to C-504; D-66 to C-504; G-67 to C-504; R-68 to C-504; T-69 to C-504; I-70 to C-504; H-71 to C-504; S-72 to C-504; G-73 to C-504; W-74 to C-504; S-75 to C-504; R-76 to C-504; F-77 to C-504; R-78 to C-504; V-79 to C-504; L-80 to C-504; P-81 to C-504; Q-82 to C-504; G-83 to C-504; L-84 to C-504; K-85 to C-504; V-86 to C-504; K-87 to C-504; Q-88 to C-504; V-89 to C-504; E-90 to C-504; R-91 to C-504; E-92 to C-504; D-93 to C-504; A-94 to C-504; G-95 to C-504; V-96 to C-504; Y-97 to C-504; V-98 to C-504; C-99 to C-504; K-100 to C-504; A-101 to C-504; T-102 to C-504; N-103 to C-504; G-104 to C-504; F-105 to C-504; G-106 to C-504; S-107 to C-504; L-108 to C-504; S-109 to C-504; V-110 to C-504; N-111 to C-504; Y-112 to C-504; T-113 to C-504; L-114 to C-504; V-115 to C-504; V-116 to C-504; L-117 to C-504; D-118 to C-504; D-119 to C-504; I-120 to C-504; S-121 to C-504; P-122 to C-504; G-123 to C-504; K-124 to C-504; E-125 to C-504; S-126 to C-504; L-127 to C-504; G-128 to C-504; P-129 to C-504; D-130 to C-504; S-131 to C-504; S-132 to C-504; S-133 to C-504; G-134 to C-504; G-135 to C-504; Q-136 to C-504; E-137 to C-504; D-138 to C-504; P-139 to C-504; A-140 to C-504; S-141 to C-504; Q-142 to C-504; Q-143 to C-504; W-144 to C-504; A-145 to C-504; R-146 to C-504; P-147 to C-504; R-148 to C-504; F-149 to C-504; T-150 to C-504; Q-151 to C-504; P-152 to C-504; S-153 to C-504; K-154 to C-504; M-155 to C-504; R-156 to C-504; R-157 to C-504; R-158 to C-504; V-159 to C-504; 1-160 to C-504; A-161 to C-504; R-162 to C-504; P-163 to C-504; V-164 to C-504; G-165 to C-504; S-166 to C-504; S-167 to C-504; V-168 to C-504; R-169 to C-504; L-170 to C-504; K-171 to C-504; C-172 to C-504; V-173 to C-504; A-174 to C-504; S-175 to C-504; G-176 to C-504; H-177 to C-504; P-178 to C-504; R-179 to C-504; P-180 to C-504; D-181 to C-504; I-182 to C-504; T-183 to C-504; W-184 to C-504; M -185 to C-504; K-186 to C-504; D-187 to C-504; D-188 to C-504; Q-189 to C-504; A-190 to C-504; L-191 to C-504; T-192 to C-504; R-193 to C-504; P-194 to C-504; E-195 to C-504; A-196 to C-504; A-197 to C-504; E-198 to C-504; P-199 to C-504; R-200 to C-504; K-201 to C-504; K-202 to C-504; K-203 to C-504; W-204 to C-504; T-205 to C-504; L-206 to C-504; S-207 to C-504; L-208 to C-504; K-209 to C-504; N-210 to C-504; L-211 to C-504; R-212 to C-504; P-213 to C-504; E-214 to C-504; D-215 to C-504; S-216 to C-504; G-217 to C-504; K-218 to C-504; Y-219 to C-504; T-220 to C-504; C-221 to C-504; R-222 to C-504; V-223 to C-504; S-224 to C-504; N-225 to C-504; R-226 to C-504; A-227 to C-504; G-228 to C-504; A-229 to C-504; I-230 to C-504; N-231 to C-504; A-232 to C-504; T-233 to C-504; Y-234 to C-504; K-235 to C-504; V-236 to C-504; D-237 to C-504; V-238 to C-504; I-239 to C-504; Q-240 to C-504; R-241 to C-504; T-242 to C-504; R-243 to C-504; S-244 to C-504; K-245 to C-504; P-246 to C-504; V-247 to C-504; L-248 to C-504; T-249 to C-504; G-250 to C-504; T-251 to C-504; H-252 to C-504; P-253 to C-504; V-254 to C-504; N-255 to C-504; T-256 to C-504; T-257 to C-504; V-258 to C-504; D-259 to C-504; F-260 to C-504; G-261 to C-504; G-262 to C-504; T-263 to C-504; T-264 to C-504; S-265 to C-504; F-266 to C-504; Q-267 to C-504; C-268 to C-504; K-269 to C-504; V-270 to C-504; R-271 to C-504; S-272 to C-504; D-273 to C-504; V-274 to C-504; K-275 to C-504; P-276 to C-504; V-277 to C-504; I-278 to C-504; Q-279 to C-504; W-280 to C-504; L-281 to C-504; K-282 to C-504; R-283 to C-504; V-284 to C-504; E-285 to C-504; Y-286 to C-504; G-287 to C-504; A-288 to C-504; E-289 to C-504; G-290 to C-504; R-291 to C-504; H-292 to C-504; N-293 to C-504; S-294 to C-504; T-295 to C-504; I-296 to C-504; D-297 to C-504; V-298 to C-504; G-299 to C-504; G-300 to C-504; Q-301 to C-504; K-302 to C-504; F-303 to C-504; V-304 to C-504; V-305 to C-504; L-306 to C-504; P-307 to C-504; T-308 to C-504; G-309 to C-504; D-310 to C-504; V-311 to C-504; W-312 to C-504; S-313 to C-504; R-314 to C-504; P-315 to C-504; D-316 to C-504; G-917 to C-504; S-318 to C-504; Y-319 to C-504; L-320 to C-504; N-321 to C-504; K-322 to C-504; L-323 to C-504; L-324 to C-504; I-325 to C-504; T-326 to C-504; R-327 to C-504; A-328 to C-504; R-329 to C-504; Q-330 to C-504; D-331 to C-504; D-332 to C-504; A-333 to C-504; G-314 to C-504; M-335 to C-504; Y-336 to C-504; I-337 to C-504; C-338 to C-504; L-339 to C-504; G-340 to C-504; A-341 to C-504; N-342 to C-504; T-343 to C-504; M-344 to C-504; G-345 to C-504; Y-346 to C-504; S-347 to C-504; F-348 to C-504; R-349 to C-504; S-350 to C-504; A-351 to C-504; F-352 to C-504; L-353 to C-504; T-354 to C-504; V-355 to C-504; L-356 to C-504; P-357 to C-504; D-358 to C-504; P-359 to C-504; K-360 to C-504; P-361 to C-504; Q-362 to C-504; G-363 to C-504; P-364 to C-504; P-365 to C-504; V-366 to C-504; A-367 to C-504; S-368 to C-504; S-369 to C-504; S-370 to C-504; S-371 to C-504; A-372 to C-504; T-373 to C-504; S-374 to C-504; L-375 to C-504; P-376 to C-504; W-377 to C-504; P-378 to C-504; V-379 to C-504; V-380 to C-504; I-381 to C-504; G-382 to C-504; I-383 to C-504; P-384 to C-504; A-385 to C-504; G-386 to C-504; A-387 to C-504; V-388 to C-504; F-389 to C-504; I-390 to C-504; L-391 to C-504; G-392 to C-504; T-393 to C-504; L-394 to C-504; L-395 to C-504; L-396 to C-504; W-397 to C-504; L-398 to C-504; C-399 to C-504; Q-400 to C-504; A-401 to C-504; Q-402 to C-504; K-403 to C-504; K-404 to C-504; P-405 to C-504; C-406 to C-504; T-407 to C-504; P-408 to C-504; A-409 to C-504; P-410 to C-504; A-411 to C-504; P-412 to C-504; P-413 to C-504; L-414 to C-504; P-415 to C-504; G-416 to C-504; H-417 to C-504; R-418 to C-504; P-419 to C-504; P-420 to C-504; G-421 to C-504; T-422 to C-504; A-423 to C-504; L-424 to C-504; D-425 to C-504; R-426 to C-504; T-427 to C-504; G-428 to C-504; D-429 to C-504; K-430 to C-504; D-431 to C-504; L-432 to C-504; P-433 to C-504; S-434 to C-504; L-435 to C-504; A-436 to C-504; A-437 to C-504; L-438 to C-504; S-439 to C-504; A-440 to C-504; G-441 to C-504; P-442 to C-504; G-443 to C-504; V-444 to C-504; G-445 to C-504; L-446 to C-504; C-447 to C-504; E-448 to C-504; E-449 to C-504; H-450 to C-504; G-451 to C-504; S-452 to C-504; P-453 to C-504; A-454 to C-504; A-455 to C-504; P-456 to C-504; Q-457 to C-504; H-458 to C-504; L-459 to C-504; L-460 to C-504; G-461 to C-504; P-462 to C-504; G-463 to C-504; P-464 to C-504; V-465 to C-504; A-466 to C-504; C-467 to C-504; P-468 to C-504; K-469 to C-504; L-470 to C-504; Y-471 to C-504; P-472 to C-504; K-473 to C-504; L-474 to C-504; Y-475 to C-504; T-476 to C-504; D-477 to C-504; I-478 to C-504; H-479 to C-504; T-480 to C-504; H-481 to C-504; T-482 to C-504; H-483 to C-504; T-484 to C-504; H-485 to C-504; S-486 to C-504; H-487 to C-504; T-488 to C-504; H-489 to C-504; S-490 to C-504; H-491 to C-504; V-492 to C-504; E-493 to C-504; G494 to C-504; K-495 to C-504; V-496 to C-504; H-497 to C-504; Q-498 to C-504; H-499 to C-504; of SEQ ID NO:2. Polynucleotides encoding these polypeptides are also encompassed by the invention.

In another embodiment, the present invention further provides polypeptides having one or more residues deleted from the amino terminus of the amino acid sequence of the extracellular portion of the FGFR5 polypeptide depicted in FIGS. 1A-C (SEQ ID NO:2) or encoded by the cDNA of the deposited clone. Particularly, in one embodiment, N-terminal deletions of the extracellular portion of the FGFR5 polypeptide can be described by the general formula m to 373, where m is an integer from 2 to 373 corresponding to the position of amino acid identified in SEQ ID NO:2, and preferably, corresponds to one of the N-terminal amino acid residues identified in the N-terminal deletions specified herein. In specific embodiments, N-terminal deletions of the extracellular portion of the FGFR5 polypeptide of the present invention comprise, or alternatively, consist of, amino acid residues: T-2 to T-373; P-3 to T-373; S-4 to T-373; P-5 to T-373; L-6 to T-373; L-7 to T-373; L-8 to T-373; L-9 to T-373; L-10 to T-373; L-11 to T-373; P-12 to T-373; P-13 to T-373; L-14 to T-373; L-15 to T-373; L-16 to T-373; G-17 to T-373; A-18 to T-373; F-19 to T-373; P-20 to T-373; P-21 to T-373; A-22 to T-373; A-23 to T-373; A-24 to T-373; A-25 to T-373; R-26 to T-373; G-27 to T-373; P-28 to T-373; P-29 to T-373; K-30 to T-373; M-31 to T-373; A-32 to T-373; D-33 to T-373; K-34 to T-373; V-35 to T-373; V-36 to T-373; P-37 to T-373; R-38 to T-373; Q-39 to T-373; V-40 to T-373; A-41 to T-373; R-42 to T-373; L-43 to T-373; G-44 to T-373; R-45 to T-373; T-46 to T-373; V-47 to T-373; R-48 to T-373; L-49 to T-373; Q-50 to T-373; C-51 to T-373; P-52 to T-373; V-53 to T-373; E-54 to T-373; G-55 to T-373; D-56 to T-373; P-57 to T-373; P-58 to T-373; P-59 to T-373; L-60 to T-373; T-61 to T-373; M-62 to T-373; W-63 to T-373; T-64 to T-373; K-65 to T-373; D-66 to T-373; G-67 to T-373; R-68 to T-373; T-69 to T-373; I-70 to T-373; H-71 to T-373; S-72 to T-373; G-73 to T-373; W-74 to T-373; S-75 to T-373; R-76 to T-373; F-77 to T-373; R-78 to T-373; V-79 to T-373; L-80 to T-373; P-81 to T-373; Q-82 to T-373; G-83 to T-373; L-84 to T-373; K-85 to T-373; V-86 to T-373; K-87 to T-373; Q-88 to T-373; V-89 to T-373; E-90 to T-373; R-91 to T-373; E-92 to T-373; D-93 to T-373; A-94 to T-373; G-95 to T-373; V-96 to T-373; Y-97 to T-373; V-98 to T-373; C-99 to T-373; K-100 to T-373; A-101 to T-373; T-102 to T-373; N-103 to T-373; G-104 to T-373; F-105 to T-373; G-106 to T-373; S-107 to T-373; L-108 to T-373; S-109 to T-373; V-110 to T-373; N-111 to T-373; Y-112 to T-373; T-113 to T-373; L-114 to T-373; V-115 to T-373; V-116 to T-373; L-117 to T-373; D-118 to T-373; D-119 to T-373; I-120 to T-373; S-121 to T-373; P-122 to T-373; G-123 to T-373; K-124 to T-373; E-125 to T-373; S-126 to T-373; L-127 to T-373; G-128 to T-373; P-129 to T-373; D-130 to T-373; S-131 to T-373; S-132 to T-373; S-133 to T-373; G-134 to T-373; G-135 to T-373; Q-136 to T-373; E-137 to T-373; D-138 to T-373; P-139 to T-373; A-140 to T-373; S-141 to T-373; Q-142 to T-373; Q-143 to T-373; W-144 to T-373; A-145 to T-373; R-146 to T-373; P-147 to T-373; R-148 to T-373; F-149 to T-373; T-150 to T-373; Q-151 to T-373; P-152 to T-373; S-153 to T-373; K-154 to T-373; M-155 to T-373; R-156 to T-373; R-157 to T-373; R-158 to T-373; V-159 to T-373; I-160 to T-373; A-161 to T-373; R-162 to T-373; P-163 to T-373; V-164 to T-373; G-165 to T-373; S-166 to T-373; S-167 to T-373; V-168 to T-373; R-169 to T-373; L-170 to T-373; K-171 to T-373; C-172 to T-373; V-173 to T-373; A-174 to T-373; S-175 to T-373; G-176 to T-373; H-177 to T-373; P-178 to T-373; R-179 to T-373; P-180 to T-373; D-181 to T-373; I-182 to T-373; T-183 to T-373; W-184 to T-373; MS-15 to T-373; K-186 to T-373; D-187 to T-373; D-178 to T-373; Q-189 to T-373; A-190 to T-373; L-191 to T-373; T-192 to T-373; R-193 to T-373; P-194 to T-373; E-195 to T-373; A-196 to T-373; A-197 to T-373; E-198 to T-373; P-199 to T-373; R-200 to T-373; K-201 to T-373; K-202 to T-373; K-203 to T-373; W-204 to T-373; T-205 to T-373; L-206 to T-373; S-207 to T-373; L-208 to T-373; K-209 to T-373; N-219 to T-373; L-211 to T-373; R-212 to T-373; P-213 to T-373; E-214 to T-373; D-215 to T-373; S-216 to T-373; G-217 to T-373; K-218 to T-373; Y-219 to T-373; T-220 to T-373; C-221 to T-373; R-222 to T-373; V-223 to T-373; S-224 to T-373; N-225 to T-373; R-226 to T-373; A-227 to T-373; G-228 to T-373; A-229 to T-373; I-230 to T-373; N-231 to T-373; A-232 to T-373; T-233 to T-373; Y-234 to T-373; K-235 to T-373; V-236 to T-373; D-237 to T-373; V-238 to T-373; I-239 to T-373; Q-240 to T-373; R-241 to T-373; T-242 to T-373; R-243 to T-373; S-244 to T-373; K-245 to T-373; P-246 to T-373; V-247 to T-373; L-248 to T-373; T-249 to T-373; G-250 to T-373; T-251 to T-373; H-252 to T-373; P-253 to T-373; V-254 to T-373; N-255 to T-373; T-256 to T-373; T-257 to T-373; V-258 to T-373; D-259 to T-373; F-260 to T-373; G-261 to T-373; G-262 to T-373; T-263 to T-373; T-264 to T-373; S-265 to T-373; F-266 to T-373; Q-267 to T-373; C-268 to T-373; K-269 to T-373; V-270 to T-373; R-271 to T-373; S-272 to T-373; D-273 to T-373; V-274 to T-373; K-275 to T-373; P-276 to T-373; V-277 to T-373; I-278 to T-373; Q-279 to T-373; W-280 to T-373; L-281 to T-373; K-282 to T-373; R-283 to T-373; V-284 to T-373; E-285 to T-373; Y-286 to T-373; G-287 to T-373; A-288 to T-373; E-289 to T-373; G-290 to T-373; R-291 to T-373; H-292 to T-373; N-293 to T-373; S-294 to T-373; T-295 to T-373; 1-296 to T-373; D-297 to T-373; V-298 to T-373; G-299 to T-373; G-300 to T-373; Q-301 to T-373; K-302 to T-373; F-303 to T-373; V-304 to T-373; V-305 to T-373; L-306 to T-373; P-307 to T-373; T-308 to T-373; G-309 to T-373; D-310 to T-373; V-311 to T-373; W-312 to T-373; S-313 to T-373; R-314 to T-373; P-315 to T-373; D-316 to T-373; G-317 to T-373; S-318 to T-373; Y-319 to T-373; L-320 to T-373; N-321 to T-373; K-322 to T-373; L-323 to T-373; L-324 to T-373; I-325 to T-373; T-326 to T-373; R-327 to T-373; A-328 to T-373; R-329 to T-373; Q-330 to T-373; D-331 to T-373; D-332 to T-373; A-333 to T-373; G-334 to T-373; M-335 to T-373; Y-336 to T-373; I-337 to T-373; C-338 to T-373; L-339 to T-373; G-340 to T-373; A-341 to T-373; N-342 to T-373; T-343 to T-373; M-344 to T-373; G-345 to T-373; Y-346 to T-373; S-347 to T-373; F-348 to T-373; R-349 to T-373; S-350 to T-373; A-351 to T-373; F-352 to T-373; L-353 to T-373; T-354 to T-373; V-355 to T-373; L-356 to T-373; P-357 to T-373; D-358 to T-373; P-359 to T-373; K-360 to T-373; P-361 to T-373; Q-362 to T-373; G-361 to T-373; P-364 to T-373; P-365 to T-373; V-366 to T-373; A-367 to T-373; S-368 to T-373; of SEQ ID NO:2.

Further embodiments of the invention are directed to C-terminal deletions of the FGFR5 polypeptide described by the general formula 1 to n, where n is an integer from I-503 corresponding to the position of amino acid residue identified in SEQ ED NO:2 and preferably, corresponds to one of the C-terminal amino acid residues identified in the C-terminal deletions specified herein. In specific embodiments, C terminal deletions of the FGFR5 polypeptide of the invention comprise, or alternatively, consist of, amino acid residues: M-1 to Q-503; M-1 to Y-502; M-1 to H-501; M-1 to I-500; M-1 to H-499; M-1 to Q-498; M-1 to H-497; M-1 to V-496; M-1 to K-4L95; M-1 to G-494; M-1 to E-493; M-1 to V-492; M-1 to H-491; M-1 to S-490; M-1 to H-489; M-1 to T-488; M-1 to H-487; M-1 to S-486; M-1 to H-485; M-1 to T-484; M-1 to H-483; M-1 to T-482; M-1 to H-481; M-1 to T-480; M-1 to H-479; M-1 to I-478; M-1 to D-477; M-1 to T-476; M-1 to Y-475; M-1 to L-474; M-1 to K-473; M-1 to P-472; M-1 to Y-471; M-1 to L-470; M-1 to K-469; M-1 to P-468; M-1 to G-467; M-1 to A-466; M-1 to V-465; M-1 to P-464; M-1 to G-463; M-1 to P-462; M-1 to G-461; M-1 to L-460; M-1 to L-459; M-1 to H-458; M-1 to Q-457; M-1 to P-456; M-1 to A-455; M-1 to A-454; M-1 to P-453; M-1 to S-452; M-1 to G-451; M-1 to H-450; M-1 to E-449; M-1 to E-448; M-1 to C-447; M-1 to L-446; M-1 to G-445; M-1 to V-444; M-1 to G-443; M-1 to P-442; M-1 to G-441; M-1 to A-440; M-1 to S-439; M-1 to L-438; M-1 to A437; M-1 to A-436; M-1 to L-435; M-1 to S-434; M-1 to P-433; M-1 to L-432; M-1 to D-431; M-1 to K-430; M-1 to D-429; M-1 to G-428; M-1 to S-427; M-1 to R-426; M-1 to D-425; M-1 to L-424; M-1 to A-423; M-1 to T-422; M-1 to G-421; M-1 to P-420; M-1 to P-419; M-1 to R-418; M-1 to H-417; M-1 to G-416; M-1 to P-415; M-1 to L-414; M-1 to P-413; M-1 to P-412; M-1 to A-411; M-1 to P-410; M-1 to A-409; M-1 to P-408; M-1 to T-407; M-1 to C-406; M-1 to P-405; M-1 to K-404; M-1 to K-403; M-1 to Q-402; M-1 to A-401; M-1 to Q-400; M-1 to C-399; M-1 to L-398; M-1 to W-397; M-1 to L-396; M-1 to L-395; M-1 to L-394; M-1 to T-393; M-1 to G-392; M-1 to L-391; M-1 to I-390; M-1 to F-389; M-1 to V-388; M-1 to A-387; M-1 to G-386; M-1 to A-385; M-1 to P-384; M-1 to 1-383; M-1 to G-382; M-1 to I-381; M-1 to V-380; M-1 to V-379; M-1 to P-378; M-1 to W-377; M-1 to P-376; M-1 to L-375; M-1 to S-374; M-1 to T-373; M-1 to A-372; M-1 to S-371; M-1 to S-370; M-1 to S-369; M-1 to S-368; M-1 to A-367; M-1 to V-366; M-1 to P-365; M-1 to P-364; M-1 to G-363; M-1 to Q-362; M-1 to P-361; M-1 to K-360; M-1 to P-359; M-1 to D-358; M-1 to P-357; M-1 to L-356; M-1 to V-355; M-1 to T-354; M-1 to L-353; M-1 to F-352; M-1 to A-351; M-1 to S-350; M-1 to R-349; M-1 to F-348; M-1 to S-347; M-1 to Y-346; M-1 to G-345; M-1 to M-344; M-1 to T-343; M-1 to N-342; M-1 to A-341; M-1 to G-340; M-1 to L-339; M-1 to C-338; M-1 to 1-337; M-1 to Y-336; M-1 to M-335; M-1 to G-334; M-1 to A-333; M-1 to D-332; M-1 to D-331; M-1 to Q-330; M-1 to R-329; M-1 to A-328; M-1 to R-327; M-1 to T-326; M-1 to I-325; M-1 to L-324; M-1 to L-323; M-1 to K-322; M-1 to N-321; M-1 to L-320; M-1 to Y-319; M-1 to S-318; M-1 to G-317; M-1 to D-316; M-1 to P-315; M-1 to R-314; M-1 to S-313; M-1 to W-312; M-1 to V-311; M-1 to D-310; M-1 to G-309; M-1 to T-308; M-1 to P-307; M-1 to L-306; M-1 to V-305; M-1 to V-304; M-1 to F-303; M-1 to K-302; M-1 to Q-301; M-1 to G-300; M-1 to G-299; M-1 to V-298; M-1 to D-297; M-1 to 1-296; M-1 to T-295; M-1 to S-294; M-1 to N-293; M-1 to H-292; M-1 to R-291; M-1 to G-290; M-1 to E-289; M-1 to A-288; M-1 to G-287; M-1 to Y-286; M-1 to E-285; M-1 to V-284; M-1 to R-283; M-1 to K-282; M-1 to L-281; M-1 to W-280; M-1 to Q-279; M-1 to I-278; M-1 to V-277; M-1 to P-276; M-1 to K-275; M-1 to V-274; M-1 to D-273; M-1 to S-272; M-1 to R-271; M-1 to V-270; M-1 to K-269; M-1 to C-268; M-1 to Q-267; M-1 to F-266; M-1 to S-265; M-1 to T-264; M-1 to T-263; M-1 to G-262; M-1 to G-261; M-1 to F-260; M-1 to D-259; M-1 to V-258; M-1 to T-257; M-1 to T-256; M-1 to N-255; M-1 to V-254; M-1 to P-253; M-1 to H-252; M-1 to T-251; M-1 to G-250; M-1 to T-249; M-1 to L-248; M-1 to V-247; M-1 to P-246; M-1 to K-245; M-1 to S-244; M-1 to R-243; M-1 to T-242; M-1 to R-241; M-1 to Q-240; M-1 to I-239; M-1 to V-238; M-1 to D-237; M-1 to V-236; M-1 to K-235; M-1 to Y-234; M-1 to T-233; M-1 to A-232; M-l to N-231; M-1 to I-230; M-1 to A-229; M-1 to G-228; M-1 to A-227; M-1 to R-226; M-1 to N-225; M-1 to S-224; M-1 to V-223; M-1 to R-222; M-1 to C-221; M-1 to T-220; M -1 to Y-219; M-1 to K-218; M-1 to G-217; M-1 to S-216; M-1 to D-215; M-1 to E-214; M-1 to P-213; M-1 to R-212; M-1 to L-211; M-1 to N-210; M-1 to K-209; M-1 to L-208; M-1 to S-207; M-1 to L-206; M-1 to T-205; M-1 to W-204; M-1 to K-203; M-1 to K-202; M-1 to K-201; M-1 to R-200; M-1 to P-199; M-1 to E-198; M-1 to A-197; M-1 to A-196; M-1 to E-195; M-1 to P-194; M-1 to R-193; M-1 to T-192; M-1 to L-191; M-1 to A-190; M-1 to Q-189; M-1 to D-188; M-1 to D-187; M-1 to K-186; M-1 to M-185; M-1 to W-184; M-1 to T-183; M-1 to I-182; M-1 to D-181; M-1 to P-180; M-1 to R-179; M-1 to P-178; M-1 to H-177; M-1 to G-176; M-1 to S-175; M-1 to A-174; M-1 to V-173; M-1 to C-172; M-1 to K-171; M-1 to L-170; M-1 to R-169; M-1 to V-168; M-1 to S-167; M-1 to S-166; M-1 to G-165; M-1 to V-164; M-1 to P-163; M-1 to R-162; M-1 to A-161; M-1 to 1-160; M-1 to V-159; M-1 to R-158; M-1 to R-157; M-1 to R-156; M-1 to M-155; M-1 to K-154; M-1 to S-153; M-1 to P-152; M-1 to Q-151; M-1 to T-150; M-1 to F-149; M-1 to R-148; M-1 to P-147; M-1 to R-146; M-1 to A-145; M-1 to W-144; M-1 to Q-143; M-1 to Q-142; M-1 to S-141; M-1 to A-140; M-1 to P-139; M-1 to D-138; M-1 to E-137; M-1 to Q-136; M-1 to G-135; M-1 to G-134; M-1 to S-133; M-1 to S-132; M-1 to S-131; M-1 to D-130; M-1 to P-129; M-1 to G-128; M-1 to L-127; M-1 to S-126; M-1 to E-125; M-1 to K-124; M-1 to G-123; M-1 to P-122; M-1 to S-121; M-1 to I-120; M-1 to D-119; M-1 to D-118; M-1 to L-117; M-1 to V-116; M-1 to V-115; M-1 to L-114; M-1 to T-113; M-1 to Y-112; M-1 to N-111; M-1 to V-110; M-1 to S-109; M-1 to L-108; M-1 to S-107; M-1 to G-106; M-1 to F-105; M-1 to G-104; M-1 to N-103; M-1 to T-102; M-1 to A-101; M-1 to K-100; M-1 to C-99; M-1 to V-98; M-1 to Y-97; M-1 to V-96; M-1 to G-95; M-1 to A-94; M-1 to D-93; M-1 to E-92; M-1 to R-91; M-1 to E-90; M-1 to V-89; M-1 to Q-88; M-1 to K-87; M-1 to V-86; M-1 to K-85; M-1 to L-84; M-1 to G-83; M-1 to Q-82; M-1 to P-81; M-1 to L-80; M-1 to V-79; M-1 to R-78; M-1 to F-77; M-1 to R-76; M-1 to S-75; M-1 to W-74; M-1 to G-73; M-1 to S-72; M-1 to H-71; M-1 to I-70; M-1 to T-69; M-1 to R-68; M-1 to G-67; M-1 to D-66; M-1 to K-65; M-1 to T-64; M-1 to W-63; M-1 to M-62; M-1 to T-61; M-1 to L-60; M-1 to P-59; M-1 to P-58; M-1 to P-57; M-1 to D-56; M-1 to G-55; M-1 to E-54; M-1 to V-53; M-1 to P-52; M-1 to C-51; M-1 to Q-50; M-1 to L-49; M-1 to R-48; M-1 to V-47; M-1 to T-46; M-1 to R-45; M-1 to G-44; M-1 to L-43; M-1 to R-42; M-1 to A-41; M-1 to V-40; M-1 to Q-39; M-1 to R-38; M-1 to P-37; M-1 to V-36; M-1 to V-35; M-1 to K-34; M-1 to D-33; M-1 to A-32; M-1 to M-31; M-1 to K-30; M-1 to P-29; M-1 to P-28; M-1 to G-27; M-1 to R-26; M-1 to A-25; M-1 to A-24; M-1 to A-23; M-1 to A-22; M-1 to P-21; M-1 to P-20; M-1 to F-19; M-1 to A-18; M-1 to G-17; M-1 to L-16; M-1 to L-15; M-1 to L-14; M-1 to P-13; M-1 to P-12; M-1 to L-11; M-1 to L-10; M-1 to L-9; M-1 to L-8; M-1 to L-7; of SEQ ID NO:2. Polynucleotides encoding these polypeptides are also encompassed by the invention.

Further embodiments of the invention are directed to C-terminal deletions of the extracellular portion of the FGFR5 polypeptide described by the general formula 1 to n, where n is an integer from 1-372 corresponding to the position of amino acid residue identified in SEQ ID NO:2 and preferably, corresponds to one of the C-terminal amino acid residues identified in the C-terminal deletions specified herein. In specific embodiments, C-terminal deletions of the extracellular portion of the FGFR5 polypeptide of the invention comprise, or alternatively, consist of, amino acid residues: A-25 to A-372; A-25 to S-371; A-25 to S-370; A-25 to S-369; A-25 to S-368; A-25 to A-367; A-25 to V-366; A-25 to P-365; A-25 to P-364; A-25 to G-363; A-25 to Q-362; A-25 to P-361; A-25 to K-360; A-25 to P-359; A-25 to D-358; A-25 to P-357; A-25 to L-356; A-25 to V-355; A-25 to T-354; A-25 to L-353; A-25 to F-352; A-25 to A-351; A-25 to S-350; A-25 to R-349; A-25 to F-348; A-25 to S-347; A-25 to Y-346; A-25 to G-345; A-25 to M-344; A-25 to T-343; A-25 to N-342; A-25 to A-341; A-25 to G-340; A-25 to L-339; A-25 to C-338; A-25 to I-337; A-25 to Y-336; A-25 to M-335; A-25 to G-334; A-25 to A-333; A-25 to D-332; A-25 to D-331; A-25 to Q-330; A-25 to R-329; A-25 to A-328; A-25 to R-327; A-25 to T-326; A-25 to I-325; A-25 to L-324; A-25 to L-323; A-25 to K-322; A-25 to N-321; A-25 to L-320; A-25 to Y-319; A-25 to S-318; A-25 to G-317; A-25 to D-316; A-25 to P-315; A-25 to R-314; A-25 to S-313; A-25 to W-312; A-25 to V-311; A-25 to D-310; A-25 to G-309; A-25 to T-308; A-25 to P-307; A-25 to L-306; A-25 to V-305; A-25 to V-304; A-25 to F-303; A-25 to K-302; A-25 to Q-301; A-25 to G-300; A-25 to G-299; A-25 to V-298; A-25 to D-297; A-25 to I-296; A-25 to T-295; A-25 to S-294; A-25 to N-293; A-25 to H-292; A-25 to R-291; A-25 to G-290; A-25 to E-289; A-25 to A-288; A-25 to G-287; A-25 to Y-286; A-25 to E-285; A-25 to V-284; A-25 to R-283; A-25 to K-282; A-25 to L-281; A-25 to W-280; A-25 to Q-279; A-25 to 1-278; A-25 to V-277; A-25 to P-276; A-25 to K-275; A-25 to V-274; A-25 to D-273; A-25 to S-272; A-25 to R-271; A-25 to V-270; A-25 to K-269; A-25 to C-268; A-25 to Q-267; A-25 to F-266; A-25 to S-265; A-25 to T-264; A-25 to T-263; A-25 to G-262; A-25 to G-261; A-25 to F-260; A-25 to D-259; A-25 to V-258; A-25 to T-257; A-25 to T-256; A-25 to N-255; A-25 to V-254; A-25 to P-253; A-25 to H-252; A-25 to T-251; A-25 to G-250; A-25 to T-249; A-25 to L-248; A-25 to V-247; A-25 to P-246; A-25 to K-245; A-25 to S-244; A-25 to R-243; A-25 to T-242; A-25 to R-241; A-25 to Q-240; A-25 to 1-239; A-25 to V-238; A-25 to D-237; A-25 to V-236; A-25 to K-235; A-25 to Y-234; A-25 to T-233; A-25 to A-232; A-25 to N-231; A-25 to 1-230; A-25 to A-229; A-25 to G-228; A-25 to A-227; A-25 to R-226; A-25 to N-225; A-25 to S-224; A-25 to V-223; A-25 to R-222; A-25 to C-221; A-25 to T-220; A-25 to Y-219; A-25 to K-218; A-25 to G-217; A-25 to S-216; A-25 to D-215; A-25 to E-214; A-25 to P-213; A-25 to R-212; A-25 to L-211; A-25 to N-210; A-25 to K-209; A-25 to L-208; A-25 to S-207; A-25 to L-206; A-25 to T-205; A-25 to W-204; A-25 to K-203; A-25 to K-202; A-25 to K-201; A-25 to R-200; A-25 to P-199; A-25 to E-198; A-25 to A-197; A-25 to A-196; A-25 to E-195; A-25 to P-194; A-25 to R-193; A-25 to T-192; A-25 to L-191; A-25 to A-190; A-25 to Q-189; A-25 to D-188; A-25 to D-187; A-25 to K-186; A-25 to M-185; A-25 to W-184; A-25 to T-183; A-25 to I-182; A-25 to D-181; A-25 to P-180; A-25 to R-179; A-25 to P-178; A-25 to H-177; A-25 to G-176; A-25 to S-175; A-25 to A-174; A-25 to V-173; A-25 to C-172; A-25 to K-171; A-25 to L-170; A-25 to R-169; A-25 to V-168; A-25 to S-167; A-25 to S-166; A-25 to G-165; A-25 to V-164; A-25 to P-163; A-25 to R-162; A-25 to A-161; A-25 to I-160; A-25 to V-159; A-25 to R-158; A-25 to R-157; A-25 to R-156; A-25 to M-155; A-25 to K-154; A-25 to S-153; A-25 to P-152; A-25 to Q-151; A-25 to T-150; A-25 to F-149; A-25 to R-148; A-25 to P-147; A-25 to R-146; A-25 to A-145; A-25 to W-144; A-25 to Q-143; A-25 to Q-142; A-25 to S-141; A-25 to A-140; A-25 to P-139; A-25 to D-138; A-25 to E-i37; A-25 to Q-136; A-25 to G-135; A-25 to G-134; A-25 to S-133; A-25 to S-132; A-25 to S-131; A-25 to D -130; A-25 to P-129; A-25 to G-128; A-25 to L-127; A-25 to S-126; A-25 to E-125; A-25 to K-124; A-25 to G-123; A-25 to P-122; A-25 to S-121; A-25 to I-120; A-25 to D-119; A-25 to D-118; A-25 to L-117; A-25 to V-116; A-25 to V-115; A-25 to L-114; A-25 to T-113; A-25 to Y-112; A-25 to N-111; A-25 to V-110; A-25 to S-109; A-25 to L-108; A-25 to S-107; A-25 to G-106; A-25 to F-105; A-25 to G-104; A-25 to N-103; A-25 to T-102; A-25 to A-101; A-25 to K-100; A-25 to C-99; A-25 to V-98; A-25 to Y-97; A-25 to V-96; A-25 to G-95; A-25 to A-94; A-25 to D-93; A-25 to E-92; A-25 to R-91; A-25 to E-90; A-25 to V-89; A-25 to Q-88; A-25 to K-87; A-25 to V-86; A-25 to K-85; A-25 to L-84; A-25 to G-83; A-25 to Q-82; A-25 to P-81; A-25 to L-80; A-25 to V-79; A-25 to R-78; A-25 to F-77; A-25 to R-76; A-25 to S-75; A-25 to W-74; A-25 to G-73; A-25 to S-72; A-25 to H-71; A-25 to I-70; A-25 to T-69; A-25 to R-68; A-25 to G-67; A-25 to D-66; A-25 to K-65; A-25 to T-64; A-25 to W-63; A-25 to M-62; A-25 to T-61; A-25 to L-60; A-25 to P-59; A-25 to P-58; A-25 to P-57; A-25 to D-56; A-25 to G-55; A-25 to E-54; A-25 to V-53; A-25 to P-52; A-25 to C-51; A-25 to Q-50; A-25 to L-49; A-25 to R-48; A-25 to V-47; A-25 to T46; A-25 to R45; A-25 to G-44; A-25 to L-43; A-25 to R-42; A-25 to A-41; A-25 to V-40; A-25 to Q-39; A-25 to R-38; A-25 to P-37; A-25 to V-36; A-25 to V-35; A-25 to K-34; A-25 to D-33; A-25 to A-32; A-25 to M-31; of SEQ ID NO:2.

Further embodiments of the invention are directed to polypeptide fragments comprising, or alternatively, consisting of, amino acids described by the general formula m to n, where m and n are integers corresponding to any one of the amino acid residues specified above for these symbols, respectively. Polynucleotides encoding such polypeptides are also provided.

Further embodiments of the invention include isolated amino acid molecules comprising a polypeptide having an amino acid sequence at least 80% identical, more preferably at least 85%, 90%, or 95% identical, and still more preferably at least 96%, 97%, 98%, or 99% identical to N and/or C terminal FGFR5 deletion fragments described supra.

It will be recognized in the art that some amino acid sequences of the FGFR5 receptors can be varied without significant effect to the structure or function of the protein. If such differences in sequence are contemplated, it should be remembered that there will be critical areas on the protein which determine activity. Thus, the invention further includes variations of the FGFR5 receptors which show substantial FGFR5 receptor activity or which include regions of FGFR5 proteins such as the polypeptide portions discussed below. Such mutants include deletions, insertions, inversions, repeats, and type substitutions. As indicated above, guidance concerning which amino acid changes are likely to be phenotypically silent can be found in Bowie et al., “Deciphering the Message in Protein Sequences: Tolerance to Amino Acid Substitutions,” Science 247:1306-1310 (1990).

Thus, the fragment, derivative or analog of the polypeptide of FIGS. 1A-C (SEQ ID NO:2), or that are encoded by the deposited cDNA, may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the FGFR5 polypeptide is fused with another compound, such as a compound to increase the half-life of the polypeptide (for example, polyethylene glycol), or (iv) one in which the additional amino acids are fused to the full length polypeptide, such as an IgG Fc fusion region peptide or leader or secretory sequence or a sequence which is employed for purification of the FGFR5 polypeptide or a proprotein sequence. Such fragments, derivatives and analogs are deemed to be within the scope of those skilled in the art from the teachings herein.

Of particular interest are substitutions of charged amino acids with another charged amino acid and with neutral or negatively charged amino acids. The latter results in polypeptides with reduced positive charge to improve the characteristics of the FGFR5 polypeptides. The prevention of aggregation is highly desirable. Aggregation of polypeptides not only results in a loss of activity but can also be problematic when preparing pharmaceutical formulations, because they can be immunogenic. (Pinckard et al., Clin. Exp. Immunol. 2:331-340 (1967); Robbins et al., Diabetes 36:838-845 (1987); Cleland et al., Crit. Rev. Therapeutic Drug Carrier Systems 10:307-377 (1993)).

The replacement of amino acids can also change the selectivity of binding to cell surface receptors. For example, Ostade et al. (Nature 361:266-268 (1993)) describes certain mutations resulting in selective binding of TNF-a to only one of the two known types of TNF receptors. Thus, the FGFR5 receptor of the present invention may include one or more amino acid substitutions, deletions or additions, either from natural mutations or human manipulation.

As indicated, changes are preferably of a minor nature, such as conservative amino acid substitutions that do not significantly affect the folding or activity of the protein (see Table 1).

TABLE 1
Conservative Amino Acid Substitutions.

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

In specific embodiments, the number of substitutions, additions or deletions in the amino acid sequence of FIGS. 1A-C (SEQ ID NO:2) and/or any of the polypeptide fragments described herein (e.g., the extracellular domains or intracellular domain) is 100, 90, 80, 75, 70, 60, 50, 40, 35, 30, 25, 20, 15, 10, 9, 8, 7, 6, 5, 4, 3, 2, 1 or 150-100, 100-50, 50-20, 20-10, 5-10, 1-5, 1-3 or 1-2.

Amino acids in the FGFR5 polypeptides of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as receptor binding in vitro. Sites that are critical for ligand-receptor binding can also be determined by structural analysis such as crystallization, nuclear magnetic resonance or photoaffinity labeling (Smith et al., J. Mol. Biol. 224:899-904 (1992) and de Vos et al., Science 255:306-312 (1992)).

The polypeptides of the present invention also include the polypeptide encoded by the deposited cDNA; the polypeptide of FIGS. 1A-C (SEQ ID NO:2); the polypeptide sequence of any of the FGFR5 domains described herein; the polypeptide sequence of FIGS. 1A-C (SEQ ID NO:2) minus a portion, or all of, one or more of the extracellular immunoglobulin domains, acid-box domain, membrane proximal domain, transmembrane domain, and intracellular domain of the FGFR5 receptor shown in FIGS. 1A-C (SEQ ID NO:2); and polypeptides which are at least 80% identical, more preferably at least 85%, 90% or 95% identical, still more preferably at least 96%, 97%, 98% or 99% identical to the polypeptides described above, and also include portions of such polypeptides with at least 30 amino acids and more preferably at least 50 amino acids.

By a polypeptide having an amino acid sequence at least, for example, 95% “identical” to a reference amino acid sequence of a FGFR5 polypeptide is intended that the amino acid sequence of the polypeptide is identical to the reference sequence except that the polypeptide sequence may include up to five amino acid alterations per each 100 amino acids of the reference amino acid of the FGFR5 receptor. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequence shown in FIGS. 1A-C (SEQ ID NO:2), the amino acid sequence encoded by the deposited cDNA clone, or fragments thereof, can be determined conventionally using known computer programs such the MegAlign program, which is included in the suite of computer applications contained within the DNASTAR program. When using Megilign or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

In a specific embodiment, the identity between a reference (query) sequence (a sequence of the present invention) and a subject sequence, also referred to as a global sequence alignment, is determined using the FASTDB computer program based on the algorithm of Brutlag et al. (Comp. App. Biosci. 6:237-245 (1990)). Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. According to this embodiment, if the subject sequence is shorter than the query sequence due to N- or C-terminal deletions, not because of internal deletions, a manual correction is made to the results to take into consideration the fact that the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. A determination of whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of this embodiment. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence. For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are made for the purposes of this embodiment.

Polynucleotides encoding polypeptides that are 80%, 85%, 90%, 95%, 96%, 97%, 98%, or 99% identical to an FGFR5 polypeptide described herein are also provided.

The polypeptides of the present invention have uses which include, but are not limited to, molecular weight marker(s) on SDS-PAGE gels or on molecular sieve gel filtration columns using methods well known to those of skill in the art.

### Detection of Disease States

Cells which express the FGFR5 polypeptides and which are believed to have a potent cellular response to fibroblast growth factor receptor family ligand include, for example, endothelial cells, mesenchymal cells, and tissues of the immune, nervous, and endocrine systems. By “a cellular response to a fibroblast growth factor receptor family ligand” is intended any genotypic, phenotypic, and/or morphologic change to a cell, cell line, tissue, tissue culture or patient that is induced by a fibroblast growth factor family receptor ligand. As indicated, such cellular responses include not only normal physiological responses to fibroblast growth factor receptor family ligands (e.g., cellular proliferation), but also diseases associated with aberrant cell growth, and aberrant cell secretion, activation, survival, migration and differentiation. In addition, Northern blots have revealed two abundantly expressed transcripts of 4 and 5-6 kb mRNA, in pancreas, heart, brain, liver, fetal liver, kidney, and skeletal muscle.

Thus, it is believed that certain tissues in mammals with certain diseases or disorders (e.g,. diseases or disorders associated with defects in the ability to effect or transduce the activities of FGFs), such as for example, diseases associated with increased or decreased cell survival, secretion, activation, migration, differentiation, and proliferation; diseases associated with the defects of wound healing, mucositis, defects of angiogenesis, immune dysfunction, endocrine dysfunction, and insulin secretion disorders; express significantly altered (e.g., enhanced or decreased) levels of the FGFR5 mRNA and polypeptide when compared to a corresponding “standard” mammal, i.e., a mammal of the same species not having the disease. Diseases associated with increased cell survival, include, for example, cancers (such as follicular lymphomas, carcinomas with p53 mutations, and hormone-dependent tumors including, but not limited to colon cancer, cardiac tumors, pancreatic cancer, melanoma, retinoblastoma, glioblastoma, lung cancer, intestinal cancer, testicular cancer, stomach cancer, neuroblastoma, myxoma, myoma, lymphoma, endothelioma, osteoblastoma, osteoclastoma, osteosarcoma, chondrosarcoma, adenoma, breast cancer, prostate cancer, Kaposi's sarcoma and ovarian cancer); autoimmune disorders (such as, multiple sclerosis, Sjogren's syndrome, Hashimoto's thyroiditis, biliary cirrhosis, Behcet's disease, Crohn's disease, polymyositis, systemic lupus erythematosus and immune-related glomerulonephritis and rheumatoid arthritis) and viral infections (such as herpes viruses, pox viruses and adenoviruses), inflammation, graft v. host disease, acute graft rejection, and chronic graft rejection.). Diseases associated with decreased cell survival, include, for example, ischemic injury (such as that caused by myocardial infarction, stroke and reperfusion injury), and disorders associated with damage to vascularized tissues (such as atherosclerosis). In preferred embodiments, FGFR5 polynucleotides, polypeptides, and/or antagonists of the invention are used to inhibit growth, progression, and/or metasis of cancers, in particular those listed above.

Additional diseases or conditions associated with increased cell survival include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Further, it is believed that altered levels of the FGFR5 polypeptide can be detected in certain body fluids (e.g., lymph, sera, plasma, urine, and spinal fluid) from mammals with the disease or disorder when compared to sera from mammals of the same species not having the disease or disorder. Thus, the invention provides a diagnostic method useful during diagnosis, which involves assaying the expression level of the gene encoding the FGFR5 polypeptide in mammalian cells or body fluid and comparing the gene expression level with a standard FGFR5 gene expression level, whereby an increase or decrease in the gene expression level over the standard is indicative of the disease or disorder.

By “assaying” the expression level of the gene encoding the FGFR5 polypeptide” is intended qualitatively or quantitatively measuring or estimating the level of the FGFR5 polypeptide or the level of the mRNA encoding the FGFR5 polypeptide in a first biological sample either directly (e.g., by determining or estimating absolute polypeptide or mRNA level) or relatively (e.g., by comparing to the FGFR5 polypeptide level or mRNA level in a second biological sample). Preferably, the FGFR5 receptor protein level or mRNA level in the first biological sample is measured or estimated and compared to a standard FGFR5 receptor protein level or mRNA level, the standard being taken from a second biological sample obtained from an individual not having the disease state. As will be appreciated in the art, once a standard FGFR5 receptor protein level or mRNA level is known, it can be used repeatedly as a standard for comparison.

By “biological sample” is intended any biological sample obtained from an individual, cell line, tissue culture, or other source which contains FGFR5 receptor protein or mRNA. Biological samples include mammalian body fluids (such as lymph, sera, plasma, urine, synovial fluid and spinal fluid), and pancreas, heart, brain, liver, skeletal muscle, kidney, and fetal liver, and other tissues. Methods for obtaining tissue biopsies and body fluids from mammals are well known in the art. Where the biological sample is to include mRNA, a tissue biopsy is the preferred source.

Where a diagnosis has already been made according to conventional methods, the present invention is useful as a prognostic indicator, whereby patients exhibiting altered FGFR5 gene expression will experience a worse clinical outcome relative to patients expressing the gene at a normal level.

Nucleic acids for diagnosis may be obtained from a biological sample of a subject, such as from blood, urine, saliva, tissue biopsy or autopsy material, using techniques known in the art. The genomic DNA may be used directly for detection or may be amplified enzymatically by using PCR or other amplification techniques prior to analysis. RNA or cDNA may also be used in similar fashion. Deletions and insertions can be detected by a change in size of the amplified product in comparison to the normal genotype. Point mutations can be identified by hybridizing amplified DNA to labeled FGFR5 nucleotide sequences. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing (see, e.g., Myers et al., Science 230:1242 (1985)). Sequence changes at specific locations may also be revealed by nuclease protection assays, such as RNase and S1 protection or the chemical cleavage method (see Cotton et al., Proc. Natl. Acad. Sci. USA 85:4397-4401 (1985)). In another embodiment, an array of oligonucleotides probes comprising FGFR5 polynucleotide sequences or fragments thereof, can be constructed to conduct efficient screening of e.g., genetic mutations. Array technology methods are well known and have general applicability and can be used to address a variety of questions in molecular genetics including gene expression, genetic linkage, and genetic variability (see for example, Chee et al., Science 274:610-613 (1996)).

The diagnostic assays offer a process for diagnosing or determining a susceptibility to specific diseases through detection of mutation in the FGFR5 gene by the methods described herein or otherwise known in the art.

In addition, specific diseases can be diagnosed by methods comprising determining from a sample derived from a subject an abnormally decreased or increased level of FGFR5 polypeptide or mRNA. Decreased or increased expression can be measured at the RNA level using any of the methods well known in the art, which include, but are not limited to, Northern blot analysis, (Harada et al., Cell 63:303-312 (1990)), S1 nuclease mapping (Fijita et al., Cell 49:357-367 (1987)), RNAse protection, the polymerase chain reaction (PCR), reverse transcription in combination with the polymerase chain reaction (RT-PCR) (Makino et al., Technique 2:295-301 (1990), reverse transcription in combination with the ligase chain reaction (RT-LCR) and other hybridization methods.

Assaying FGFR5 polypeptide levels in a biological sample can be by any techniques known in the art, which include, but are not limited to, radioimmunoassays, competitive-binding assays, Western Blot analysis and enzyme linked immunosorbent assays (ELISAs) and other antibody-based techniques. For example, FGFR5 polypeptide expression in tissues can be studied with classical immunohistological methods (Jalkanen et al., J. Cell. Biol. 101:976-985 (1985); Jalkanen et al., J. Cell. Biol. 105:3087-3096 (1987)).

Suitable labels are known in the art and include enzyme labels, such as, Glucose oxidase, and radioisotopes, such as iodine (¹²⁵I, ¹²¹I), carbon (¹⁴C), sulfur (³⁵S), tritium (³H), indium (¹²In), and technetium (^{99m}Tc), and fluorescent labels, such as fluorescein and rhodamine, and biotin.

### Antibodies

The present invention further relates to antibodies and T-cell antigen receptors (TCR) which specifically bind the polypeptides of the present invention. The antibodies of the present invention include IgG (including IgG1, IgG2, IgG3, and IgG4), IgA (including IgA1 and IgA2), IgD, IgE, or IgM, and IgY. As used herein, the term “antibody” (Ab) is meant to include whole antibodies, including single-chain whole antibodies, and antigen-binding fragments thereof. Most preferably the antibodies are human antigen binding antibody fragments of the present invention include, but are not limited to, Fab, Fab′ and F(ab′)2, Fd, single-chain Fvs (scFv), single-chain antibodies, disulfide-linked Fvs (sdFv) and fragments comprising either a V_{L} or V_{H} domain. The antibodies may be from any animal origin including birds and mammals. Preferably, the antibodies are human, murine, rabbit, goat, guinea pig, camel, horse, or chicken.

Antigen-binding antibody fragments, including single-chain antibodies, may comprise the variable region(s) alone or in combination with the entire or partial of the following: hinge region, CH1, CH2, and CH3 domains. Also included in the invention are any combinations of variable region(s) and hinge region, CH1, CH2, and CH3 domains. The present invention further includes chimeric, humanized, and human monoclonal and polyclonal antibodies which specifically bind the polypeptides of the present invention. The present invention further includes antibodies which are anti-idiotypic to the antibodies of the present invention.

The antibodies of the present invention may be monospecific, bispecific, trispecific or of greater multispecificity. Multispecific antibodies may be specific for different epitopes of a polypeptide of the present invention or may be specific for both a polypeptide of the present invention as well as for heterologous compositions, such as a heterologous polypeptide or solid support material. See, e.g., WO 93/17715; WO 92/08802; WO 91/00360; WO 92/05793; Tutt, A. et al. (1991) J. Immunol. 147:60-69; U.S. Pat. Nos. 5,573,920, 4,474,893, 5,601,819, 4,714,681, 4,925,648; Kostelny, S. A. et al. (1992) J. Immunol. 148:1547-1553.

Antibodies of the present invention may be described or specified in terms of the epitope(s) or portion(s) of a polypeptide of the present invention which are recognized or specifically bound by the antibody. The epitope(s) or polypeptide portion(s) may be specified as described herein, e.g., by N-terminal and C-terminal positions, by size in contiguous amino acid residues, or listed in the Tables and Figures. Antibodies which specifically bind any epitope or polypeptide of the present invention may also be excluded. Therefore, the present invention includes antibodies that specifically bind polypeptides of the present invention, and allows for the exclusion of the same.

Antibodies of the present invention may also be described or specified in terms of their cross-reactivity. Antibodies that do not bind any other analog, ortholog, or hormolog of the polypeptides of the present invention are included. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present invention are also included in the present invention. Further included in the present invention are antibodies which only bind polypeptides encoded by polynucleotides which hybridize to a polynucleotide of the present invention under stringent hybridization conditions (as described herein). Antibodies of the present invention may also be described or specified in terms of their binding affinity. Preferred binding affinities include those with a dissociation constant or Kd less than 5×10⁻⁶M, 10⁻⁶M, 5×10⁻⁷M, 10⁻⁷M, 5×10⁻⁸M, 10⁻⁸M, 5×10^{- 9}M, 10⁻⁹M, 5×10⁻¹⁰M, 10⁻¹⁰M, 5×10⁻¹¹M, 10⁻¹¹M, 5×10⁻¹²M, 10⁻¹²M, 5×10⁻³M, 10⁻³M, 5×10⁻¹⁴M, 10⁻¹⁴M, 5×10⁻¹⁵M, and 10⁻¹⁵M.

Antibodies of the present invention have uses that include, but are not limited to, methods known in the art to purify, detect, and target the polypeptides of the present invention including both in vitro and in vivo diagnostic and therapeutic methods. For example, the antibodies have use in immunoassays for qualitatively and quantitatively measuring levels of the polypeptides of the present invention in biological samples. See, e.g., Harlow et al., ANTIBODIES: A LABORATORY MANUAL, (Cold Spring Harbor Laboratory Press, 2nd ed. 1988) (incorporated by reference in the entirety).

The antibodies of the present invention may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, or toxins. See, e.g., WO 92/08495; WO 91/14438; WO 89/12624; U.S. Pat. No. 5,314,995; and EP 0 396 387.

The antibodies of the present invention may be prepared by any suitable method known in the art. For example, a polypeptide of the present invention or an antigenic fragment thereof can be administered to an animal in order to induce the production of sera containing polyclonal antibodies. Monoclonal antibodies can be prepared using a wide of techniques known in the art including the use of hybridoma and recombinant technology. See, e.g., Harlow et al., ANTIBODIES: A LABORATORY MANUAL, (Cold Spring Harbor Laboratory Press, 2nd ed. 1988); Hammerling, et al., in: MONOCLONAL ANTIBODIES AND T-CELL HYBRIDOMAS 563-681 (Elsevier, N.Y., 1981) (said references incorporated by reference in their entireties). Fab and F(ab′)2 fragments may be produced by proteolytic cleavage, using enzymes such as papain (to produce Fab fragments) or pepsin (to produce F(ab′)2 fragments).

Alternatively, antibodies of the present invention can be produced through the application of recombinant DNA technology or through synthetic chemistry using methods known in the art. For example, the antibodies of the present invention can be prepared using various phage display methods known in the art. In phage display methods, functional antibody domains are displayed on the surface of a phage particle which carries polynucleotide sequences encoding them. Phage with a desired binding property are selected from a repertoire or combinatorial antibody library (e.g. human or murine) by selecting directly with antigen, typically antigen bound or captured to a solid surface or bead. Phage used in these methods are typically filamentous phage including fd and M13 with Fab, Fv or disulfide stabilized Fv antibody domains recombinantly fused to either the phage gene III or gene VIII protein. Examples of phage display methods that can be used to make the antibodies of the present invention include those disclosed in Brinkman U. et al. (1995) J. Immunol. Methods 182:41-50; Ames, R. S. et al. (1995) J. Immunol. Methods 184:177-186; Kettleborough, C. A. et al. (1994) Eur. J. Immunol. 24:952-958; Persic, L. et al. (1997) Gene 187 9-18; Burton, D. R. et al. (1994) Advances in Immunology 57:191-280; PCT/GB91/01134; WO 90/02809; WO 91/10737; WO 92/01047; WO 92/18619; WO 93/11236; WO 95/15982; WO 95/20401; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743 (said references incorporated by reference in their entireties).

As described in the above references, after phage selection, the antibody coding regions from the phage can be isolated and used to generate whole antibodies, including human antibodies, or any other desired antigen binding fragment, and expressed in any desired host including mammalian cells, insect cells, plant cells, yeast, and bacteria. For example, techniques to recombinantly produce Fab, Fab′ and F(ab′)2 fragments can also be employed using methods known in the art such as those disclosed in WO 92/22324; Mullinax, R. L. et al. (1992) BioTechniques 12(6):864-869; and Sawai, H. et al. (1995) AJRI 34:26-34; and Better, M. et al. (1988) Science 240:1041-1043 (said references incorporated by reference in their entireties).

Examples of techniques which can be used to produce single-chain Fvs and antibodies include those described in U.S. Pat. Nos. 4,946,778 and 5,258,498; Huston et al. (1991) Methods in Enzymology 203:46-88; Shu, L. et al. (1993) PNAS 90:7995-7999; and Skerra, A. et al. (1988) Science 240:1038-1040. For some uses, including in vivo use of antibodies in humans and in vitro detection assays, it may be preferable to use chimeric, humanized, or human antibodies. Methods for producing chimeric antibodies are known in the art. See e.g., Morrison, Science 229:1202 (1985); Oi et al., BioTechniques 4:214 (1986); Gillies, S. D. et al. (1989) J. Immunol. Methods 125:191-202; and U.S. Pat. No. 5,807,715. Antibodies can be humanized using a variety of techniques including CDR-grafting (EP 0 239 400; WO 91/09967; U.S. Pat. Nos. 5,530,101; and 5,585,089), veneering or resurfacing (EP 0 592 106; EP 0 519 596; Padlan E. A., (1991) Molecular Immunology 28(4/5):489-498; Studnicka G. M. et al. (1994) Protein Engineering 7(6):805-814; Roguska M.A. et al. (1994) PNAS 91:969-973), and chain shuffling (U.S. Pat. No. 5,565,332). Human antibodies can be made by a variety of methods known in the art including phage display methods described above. See also, U.S. Pat. Nos. 4,444,887, 4,716,111, 5,545,806, and 5,814,318; and WO 98/46645 (said references incorporated by reference in their entireties).

Further included in the present invention are antibodies recombinantly fused or chemically conjugated (including both covalently and non-covalently conjugations) to a polypeptide of the present invention. The antibodies may be specific for antigens other than polypeptides of the present invention. For example, antibodies may be used to target the polypeptides of the present invention to particular cell types, either in vitro or in vivo, by fusing or conjugating the polypeptides of the present invention to antibodies specific for particular cell surface receptors. Antibodies fused or conjugated to the polypeptides of the present invention may also be used in in vitro immunoassays and purification methods using methods known in the art. See e.g., Harbor et al. supra and WO 93/21232; EP 0 439 095; Naramura, M. et al. (1994) Immunol. Lett. 39:91-99; U.S. Pat. No. 5,474,981; Gillies, S. O. et al. (1992) PNAS 89:1428-1432; Fell, H. P. et al. (1991) J. Immunol. 146:2446-2452 (said references incorporated by reference in their entireties).

The present invention further includes compositions comprising the polypeptides of the present invention fused or conjugated to antibody domains other than the variable regions. For example, the polypeptides of the present invention may be fused or conjugated to an antibody Fc region, or portion thereof. The antibody portion fused to a polypeptide of the present invention may comprise the hinge region, CH1 domain, CH2 domain, and CH3 domain or any combination of whole domains or portions thereof. The polypeptides of the present invention may be fused or conjugated to the above antibody portions to increase the in vivo half life of the polypeptides or for use in immunoassays using methods known in the art. The polypeptides may also be fused or conjugated to the above antibody portions to form multimers. For example, Fc portions fused to the polypeptides of the present invention can form dimers through disulfide bonding between the Fc portions. Higher multimeric forms can be made by fusing the polypeptides to portions of IgA and IgM. Methods for fusing or conjugating the polypeptides of the present invention to antibody portions are known in the art. See e.g., U.S. Pat. Nos. 5,336,603, 5,622,929, 5,359,046, 5,349,053, 5,447,851, 5,112,946; EP 0 307 434, EP 0 367 166; WO 96/04388, WO 91/06570; Ashkenazi, A. et al. (1991) PNAS 88:10535-10539; Zheng, X. X. et al. (1995) J. Immunol. 154:5590-5600; and Vil, H. et al. (1992) PNAS 89:11337-11341 (said references incorporated by reference in their entireties).

The invention further relates to antibodies that act as agonists or antagonists of the polypeptides of the present invention. Antibodies which act as agonists or antagonists of the polypeptides of the present invention include, for example, antibodies which disrupt receptor/ligand interactions with the polypeptides of the invention either partially or fully. For example, the present invention includes antibodies that disrupt the ability of the proteins of the invention to binds FGFR5 ligand(s). In another example, the present invention includes antibodies which allow the proteins of the invention to multimerize, but disrupts the ability of the proteins of the invention to bind one or more FGFR5 ligand(s). In yet another example, the present invention includes antibodies which allow the proteins of the invention to bind FGFR5 ligand(s), but blocks biological activity associated with the FGFR5 receptor.

Antibodies which act as agonists or antagonists of the polypeptides of the present invention also include, both receptor-specific antibodies and ligand-specific antibodies. Included are receptor-specific antibodies that do not prevent ligand binding but prevent receptor activation. Receptor activation (i.e., signaling) may be determined by techniques described herein or otherwise known in the art. Also included are receptor-specific antibodies which both prevent ligand binding and receptor activation. Likewise, included are neutralizing antibodies which bind the ligand and prevent binding of the ligand to the receptor, as well as antibodies which bind the ligand, thereby preventing receptor activation, but do not prevent the ligand from binding the receptor. Further included are antibodies that activate the receptor. These antibodies may act as agonists for either all or less than all of the biological activities affected by ligand-mediated receptor activation. The antibodies may be specified as agonists or antagonists for biological activities comprising specific activities disclosed herein. The above antibody agonists can be made using methods known in the art. See e.g., WO 96/40281; U.S. Pat. No. 5,811,097; Deng, B. et al., Blood 92(6):1981-1988 (1998); Chen, Z. et al., Cancer Res. 58(16):3668-3678 (1998); Harrop, J. A. et al., J. Immunol. 161(4):1786-1794 (1998); Zhu, Z. et al., Cancer Res. 58(15):3209-3214 (1998); Yoon, D.Y. et al., J. Immunol. 160(7):3170-3179 (1998); Prat, M. et al., J. Cell. Sci. 111(Pt2):237-247 (1998); Pitard, V. et al., J. Immunol. Methods 205(2):177-190 (1997); Liautard, J. et al., Cytokinde 9(4):233-241 (1997); Carlson, N. G. et al., J. Biol. Chem. 272(17):11295-11301 (1997); Taryman, R. E. et al., Neuron 14(4):755-762 (1995); Muller, Y. A. et al., Structure 6(9):1153-1167 (1998); Bartunek, P. et al., Cytokine 8(1):14-20 (1996)(said references incorporated by reference in their entireties).

As discussed above, antibodies to the FGFR5 protein of the invention can, in turn, be utilized to generate anti-idiotype antibodies that “mimic” FGFR5 using techniques well known to those skilled in the art. (See, e.g., Greenspan & Bona, FASEB J. 7(5):437-444; (1989) and Nissinoff, J. Immunol. 147(8):2429-2438 (1991)). For example, antibodies which bind to FGFR5 and competitively inhibit FGFR5 multimerization and/or binding to ligand can be used to generate anti-idiotypes that “mimic” the FGFR5 mutimerization and/or binding domain and, as a consequence, bind to and neutralize FGFR5 and/or its ligand. Such neutralizing anti-idiotypes or Fab fragments of such anti-idiotypes can be used in therapeutic regimens to neutralize FGFR5 ligand. For example, such anti-idiotypic antibodies can be used to bind FGFR5, or to bind FGFR5 ligands/receptors, and thereby block FGFR5 biological activity.

### Agonists and Antagonists of FGFR5

In one embodiment, the present invention is directed to a method for identifying compounds that interact with (e.g., bind to) FGFR5 polypeptides (including, but not limited to full length FGFR5, and the extracellular and/or intracellular domain of FGFR5). Compounds identified may be useful, for example, in modulating the activity of FGFR5 gene products; in elaborating the biological function of FGFR5; in screens for identifying compounds that disrupt normal FGFR5 interactions; or may in themselves disrupt such interactions and therefore may have uses which include, for example, as analgesic agents, regulators of hematopoiesis, or as regulators of immune response.

The principle of the assays used to identify compounds that bind to FGFR5 involves preparing a reaction mixture of FGFR5 and the test compound under conditions and for a time sufficient to allow the two components to interact and bind, thus forming a complex which can be removed and/or detected in the reaction mixture. The FGFR5 polypeptide species used can vary depending upon the goal of the screening assay. For example, where agonists of the natural ligand are sought, the full length FGFR5, or a soluble truncated FGFR5 (e.g., containing one or more extracellular and/or intracellular domains, but in which the transmembrane domain is deleted from the molecule, a peptide corresponding to a FGFR5 extracellular domain or a protein containing a FGFR5 extracellular domain fused to a polypeptide that affords advantages in the assay system (e.g., labeling, isolation of the resulting complex, etc.) can be utilized. Where compounds that interact with the FGFR5 intracellular domain are sought to be identified, peptides corresponding to the FGFR5 intracellular domain and fusion proteins containing a FGFR5 intracellular domain can be used.

The compounds that may be screened in accordance with the invention include, but are not limited to, soluble peptides, including but not limited to those found in random peptide libraries; (see, e.g., Lam et al., Nature 354:82-84 (1991); Houghten, R. et al., Nature 354:84-86 (1991)), cell or tissue lysates, and biological samples (e.g,. cells, tissue, sera and lymph). Such compounds may also be found in random peptide expression libraries, and genomic or cDNA expression libraries, or combinatorial chemistry-derived molecular libraries made of D- and/or L-configuration amino acids; phosphopeptides (including, but not limited to members of random or partially degenerate, directed phosphopeptide libraries; see, e.g., Songyang et al., Cell 72:767-778 (1993)); antibodies (including, but not limited to, polyclonal, monoclonal, humanized, anti-idiotypic, chimeric or single chain antibodies, and FAb, F(ab′)2 and FAb expression library fragments, and epitope-binding fragments thereof); and small organic or inorganic molecules.

Numerous experimental methods may be used to select and detect polypeptides that bind with FGFR5, including, but not limited to, protein affinity chromatography, affinity blotting, immunoprecipitation, cross-linking, and library based methods such as protein probing, phage display and the two-hybrid system. See generally, Phizicky et al., Microbiol. Rev. 59:94-123 (1995). Once isolated, such an FGFR5-binding polypeptide can be identified and can, in turn, be used, in conjunction with standard techniques, to identify polypeptides with which it interacts. For example, at least a portion of the amino acid sequence of a polypeptide that interacts with FGFR5 can be ascertained using techniques well known to those of skill in the art, such as via the Edman degradation technique. (See, e.g., Creighton, 1983, “Proteins: Structures and Molecular Principles”, W. H. Freeman & Co., N.Y., pp.34-49). The amino acid sequence obtained may be used as a guide for the generation of oligonucleotide mixtures that can be used to screen for gene sequences encoding such polypeptides. Screening may be accomplished, for example, by standard hybridization or PCR techniques. Techniques for the generation of oligonucleotide mixtures and the screening are well-known. (See, e.g., Sambrook et al., Molecular Cloning: A Laboratory Manual, 2nd Edit., (1989), Cold Spring Harbor Laboratory Press; and PCR Protocols: A Guide to Methods and Applications, 1990, Innis, M. et al., eds. Academic Press, Inc., New York).

Additionally, methods may be employed which result in the simultaneous identification of genes which encode polypeptides interacting with FGFR5. These methods include, for example, probing expression libraries, in a manner similar to the well known technique of antibody probing of λgt11 libraries, using labeled FGFR5 polypeptide, such as a FGFR5 fusion protein wherein a FGFR5 domain is fused to a marker (e.g., an enzyme, fluor, luminescent protein, or dye), or an Ig-Fc domain. For example, the two-hybrid system may be used to detect interaction between FGFR5 and candidate proteins for which genes encoding the candidate polypeptides are available by constructing the appropriate hybrids and testing for reporter gene activity. If an interaction is detected using the two-hybrid method, deletions can be made in the DNA encoding the candidate interacting polypeptide or the FGFR5 polypeptide to identify a minimal domain for interaction. Alternatively, the two-hybrid system can be used to screen available organismal and/or mammalian tissue specific libraries of activation domain hybrids to identify polypeptides that bind to a FGFR5 polypeptide. These screens result in the immediate availability of the cloned gene for any new polypeptide identified. In addition, since multiple clones that encode overlapping regions of protein are often identified, the minimal domain for interaction may be readily apparent from the initial screen.

Assays may also be used that identify compounds which bind to FGFR5 gene regulatory sequences (e.g., promoter or enhancer sequences) and which may modulate FGFR5 gene expression. See e.g., Platt, J. Biol. Chem. 269:28558-28562 (1994), which is incorporated herein by reference in its entirety.

The screening assays can be conducted in a variety of ways. For example, one method to conduct such an assay would involve anchoring the FGFR5 polypeptide (e.g., fusion protein) or the test substance onto a solid phase and detecting FGFR5/test compound complexes anchored on the solid phase at the end of the reaction. In one embodiment of such a method, the FGFR5 reactant may be anchored onto a solid surface, and the test compound, which is not anchored, may be labeled, either directly or indirectly.

In practice, microtiter plates may conveniently be utilized as the solid phase. The anchored component may be immobilized by non-covalent or covalent attachments. Non-covalent attachment may be accomplished by simply coating the solid surface with a solution of the polypeptide and drying. Alternatively, an immobilized antibody, preferably a monoclonal antibody, specific for the polypeptide to be immobilized may be used to anchor the polypeptide to the solid surface. The surfaces may be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for FGFR5 polypeptide or fusion protein or the test compound to anchor any complexes formed in solution, and a labeled antibody specific for the other component of the possible complex to detect anchored complexes.

Alternatively, cell-based systems can be used to identify compounds which may interact with FGFR5 and/or act to ameliorate symptoms or disorders associated with aberrant FGFR5 concentrations or compositions. Such cell systems can include, for example, recombinant or non-recombinant cells, such as cell lines, which express FGFR5. For example, pancreas, heart, brain, liver, skeletal muscle, kidney, and fetal liver cells, or cell lines derived from pancreas, heart, brain, liver, skeletal muscle, kidney, and fetal liver can be used. In addition, expression host cells (e.g., COS cells, CHO cells, fibroblasts) genetically engineered to express a functional FGFR5 and to respond to activation by an FGF liand (e.g., as measured by a chemical or phenotypic change, induction of another host cell gene, change in ion flux (e.g., Ca⁺⁺), hydolysis of polyphosphoinositides , etc.), can be used as an end point in the assay.

In utilizing such cell systems, cells may be exposed to a test compound, at a sufficient concentration and for a time sufficient to elicit a response in the exposed cells. After exposure, the cells can be assayed to measure alterations in the expression of the FGFR5 gene, for example, by assaying cell lysates for FGFR5 mRNA transcripts (e.g., by Northern analysis), or for FGFR5 protein expressed in the cell; compounds which regulate or modulate expression of the FGFR5 gene are good candidates as therapeutics.

Cellular responses that may be assayed according to this embodiment, include, but are not limited to alterations in the expression of the FGFR5 gene, e.g., by assaying cell lysates for FGFR5 mRNA transcripts (e.g., by Northern analysis) or for FGFR5 expressed in the cell; compounds which regulate or modulate expression of the FGFR5 gene are good candidates as therapeutics. Additionally, activity of the FGFR5 signal transduction pathway itself (e.g., cation flux, such as calcium flux) can be routinely assayed using techniques known in the art (see, e.g,. Caterina et al., Nature 389:816-824 (1997), the contents of which are herein incorporated by reference in its entirety). Additional cellular activities associated with the FGFR5 signal transduction pathway that may be routinely assayed include changes in intracellular pH levels (Tsuda et al., FEBS Lett. 187:43-46(1985), increased hydolysis of polyphosphoinositides (Brown et al., FEBS Lett. 247:227-231(1989), increased phosphorylation of cellular proteins (Huang and Huang, J. Biol. Chem., 261:9568-9571(1986), Pelech et al., Proc. Natl. Acad. Sci. U.S.A., 83:5968-5972(1986), Coughlin et al., J. Biol. Chem., 263:988-993(1988)), and increased transcription of a subset of cellular genes, including c-myc and c-fos (Kruijer et al., Nature(London) 312:711-716(1984), Muller et al., Nature(London) 312:716-720(1984), Stumpo and Blackshear, Proc. Natl. Acad. Sci. U.S.A. 83:9453-9457(1986)), which are incorporated herein by reference.

In another embodiment, the FGFR5 signal transduction pathway can be assayed. For example, after exposure, the cell lysates can be assayed for the presence of hydolysis (hydrolytic activity) of polyphosphoinositides, as compared to lysates derived from unexposed control cells. The ability of a test compound to inhibit the hydolysis (hydrolytic activity) of polyphosphoinositides in these assay systems indicates that the test compound inhibits signal transduction initiated by FGFR5 activation. In yet another approach, ion flux, such as calcium ion flux, can be measured as an end point for FGFR5 stimulated signal transduction.

In another embodiment, the present invention is directed to a method for inhibiting an activity (e.g., hydolysis of polyphosphoinositides), of FGFR5 induced by a FGFR5 ligand, which involves administering to a cell which expresses a FGFR5 polypeptide, an effective amount of a FGFR5 receptor ligand, analog or an antagonist capable of decreasing FGFR5 mediated signaling. Preferably, FGFR5 receptor mediated signaling is decreased to treat a disease wherein increased tissue proliferation is exhibited. An antagonist can include soluble forms of the FGFR5 and antibodies directed against the FGFR5 polypeptides which block FGFR5 receptor mediated signaling. Preferably, FGFR5 receptor mediated signaling is decreased to treat a disease, or to decrease survival, secretion, proliferation, migration and/or differentiation of cells.

In an additional embodiment, the present invention is directed to a method for increasing an activity (e.g., hydolysis of polyphosphoinositides), induced by a FGFR5 ligand (e.g., members of the FGF family), which involves administering to a cell which expresses a FGFR5 polypeptide an effective amount of an agonist capable of increasing FGFR5 receptor mediated signaling. Preferably, FGFR5 receptor mediated signaling is increased to treat a disease wherein decreased fibroblast cell growth is exhibited. Agonists of the present invention include monoclonal antibodies directed against the FGFR5 polypeptides which stimulate FGFR5 receptor mediated signaling. Preferably, FGFR5 receptor mediated signaling is increased to treat a disease, and to increase survival, secretion, proliferation, migration, and/or differentiation of cells.

By “agonist” is intended naturally occurring and synthetic compounds capable of eliciting or enhancing fibroblast cell growth mediated by FGFR5 polypeptides. Such agonists include agents which increase expression of FGFR5 receptors or increase the sensitivity of the expressed receptor. By “antagonist” is intended naturally occurring and synthetic compounds capable of inhibiting or decreasing FGFR5 mediated fibroblast cell growth. Such antagonists include agents which decrease expression of FGFR5 receptors or decrease the sensitivity of the expressed receptor. Whether any candidate “agonist” or “antagonist” of the present invention can enhance or inhibit a FGFR5 mediated cellular response, such as, for example, hydolysis of polyphosphoinositides, and cell proliferation,. survival, and differentiation can be determined using art-known ligand/receptor cellular response assays, and ion flux assays, including those described herein.

Thus, the present invention also provides a screening method for identifying compounds capable of enhancing or inhibiting a cellular response induced by FGFR5 receptors. The method involves contacting cells which express FGFR5 polypeptides with the candidate compound in the presence of a FGFR5 ligand (e.g., a member of the FGF family), assaying a cellular response (e.g., hydolysis of polyphosphoinositides), and comparing the cellular response to a standard cellular response, the standard being assayed when contact is made between the FGFR5 ligand and FGFR5, an increased cellular response over the standard indicates that the compound is an agonist of the FGFR5-mediated signaling pathway and a decreased cellular response over the standard indicates that the compound is an antagonist of the FGFR5-mediated signaling pathway. By “assaying a cellular response” is intended qualitatively or quantitatively measuring a cellular response to a candidate compound and/or a FGFR5 (e.g., hydolysis of polyphosphoinositides). By the invention, a cell expressing a FGFR5 polypeptide can be contacted with either an endogenous or exogenously administered FGFR5 ligand.

One such screening technique involves the use of cells which express the receptor (for example, transfected kidney-derived HEK293 cells) in a system which measures tyrosine phosphorylation changes caused by receptor activation, for example, as described Caterina et al., Nature, 389:816-824 (1997). For example, compounds may be contacted with a cell which expresses the FGFR5 polypeptide of the present invention and tyrosine phosphorylation may be measured to determine whether the potential compound activates (i.e., leads to elevated hydolysis activity of polyphosphoinositides) or inhibits the receptor.

Another method involves screening for compounds which inhibit activation of the receptor polypeptide of the present invention (i.e., antagonists) by determining inhibition of binding of labeled ligand to cells which have the receptor on the surface thereof. Such a method involves transfecting a eukaryotic cell with DNA encoding the FGFR5 polypeptide such that the cell -expresses the receptor on its surface and contacting the cell with a compound in the presence of a labeled form of a FGFR5 ligand (e.g., a member of the FGF family). The ligand can be labeled, e.g., by radioactivity. The amount of labeled ligand bound to the receptors is measured, e.g., by measuring radioactivity of the FGFR5 polypeptide. If the compound binds to the receptor as determined by a reduction of labeled ligand which binds to the receptors, the binding of labeled ligand to the FGFR5 polypeptide is inhibited.

Soluble forms of the polypeptides of the present invention may be utilized in the ligand binding assay described above. These forms of the FGFR5 receptor are contacted with ligands in the extracellular medium after they are secreted. A determination is then made as to whether the secreted protein will bind to FGFR5 receptor ligands.

Agonists according to the present invention include compounds such as, for example, fibroblast growth factor receptor ligand peptide fragments. Preferred agonists include FGFR5 polypeptide fragments of the invention and/or polyclonal and monoclonal antibodies raised against a FGFR5 polypeptide, or a fragment thereof.

FGFR5 polypeptides and polynucleotides and compounds identified as FGFR5 agonists or antagonists using assays described herein or otherwise known in the art, have uses which include, but are not limited to, treating diseases, regulating hematopoiesis, regulating immune responses, regulating cell survival, activation, secretion, migration and differentiation, regulating mucositis, regulating angiogenesis, regulating endocrine function, regulating insulin secretion, and in furthering our understanding of the effects of fibroblast growth factors and their receptors on the differentiation and proliferation of any number of tissues mentioned previously.

### Gene Therapy Methods

Another aspect of the present invention is to gene therapy methods for treating disorders, diseases and conditions. The gene therapy methods relate to the introduction of nucleic acid (DNA, RNA and antisense DNA or RNA) sequences into an animal to achieve expression of the FGFR5 polypeptide of the present invention. This method requires a polynucleotide which codes for a FGFR5 polypeptide operatively linked to a promoter and any other genetic elements necessary for the expression of the polypeptide by the target tissue. Such gene therapy and delivery techniques are known in the art, see, for example, WO90/11092, which is herein incorporated by reference.

Thus, for example, cells from a patient may be engineered with a polynucleotide (DNA or RNA) comprising a promoter operably linked to a FGFR5 polynucleotide ex vivo, with the engineered cells then being provided to a patient to be treated with the polypeptide. Such methods are well-known in the art. For example, see Belldegrun, A., et al., J. Natl. Cancer Inst. 85: 207-216 (1993); Ferrantini, M. et al., Cancer Research 53: 1107-1112 (1993); Ferrantini, M. et al., J. Immunology 153: 4604-4615 (1994); Kaido, T., et al., Int. J. Cancer 60: 221-229 (1995); Ogura, H., et al., Cancer Research 50: 5102-5106 (1990); Santodonato, L., et al., Human Gene Therapy 7:1-10 (1996); Santodonato, L., et al., Gene Therapy 4:1246-1255 (1997); and Zhang, J.-F. et al., Cancer Gene Therapy, 3: 31-38 (1996)), which are herein incorporated by reference. In one embodiment, the cells which are engineered are arterial cells. The arterial cells may be reintroduced into the patient through direct injection to the artery, the tissues surrounding the artery, or through catheter injection.

As discussed in more detail below, the FGFR5 polynucleotide constructs can be delivered by any method that delivers injectable materials to the cells of an animal, such as, injection into the interstitial space of tissues (heart, muscle, skin, lung, liver, and the like). The FGFR5 polynucleotide constructs may be delivered in a pharmaceutically acceptable liquid or aqueous carrier.

In one embodiment, the FGFR5 polynucleotide is delivered as a naked polynucleotide. The term “naked” polynucleotide, DNA or RNA refers to sequences that are free from any delivery vehicle that acts to assist, promote or facilitate entry into the cell, including viral sequences, viral particles, liposome formulations, lipofectin or precipitating agents and the like. However, the FGFR5 polynucleotides can also be delivered in liposome formulations and lipofectin formulations and the like can be prepared by methods well known to those skilled in the art. Such methods are described, for example, in U.S. Pat. Nos. 5,593,972, 5,589,466, and 5,580,859, which are herein incorporated by reference.

The FGFR5 polynucleotide vector constructs used in the gene therapy method are preferably constructs that will not integrate into the host genome nor will they contain sequences that allow for replication. Appropriate vectors include pWLNEO, pSV2CAT, pOG44, pXT1 and pSG available from Stratagene; pSVK3, pBPV, pMSG and pSVL available from Pharmacia; and pEF1/V5, pcDNA3.1, and pRc/CMV2 available from Invitrogen. Other suitable vectors will be readily apparent to the skilled artisan.

Any strong promoter known to those skilled in the art can be used for driving the expression of FGFR5 DNA. Suitable promoters include adenoviral promoters, such as the adenoviral major late promoter; or heterologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs; the b-actin promoter; and human growth hormone promoters. The promoter also may be the native promoter for FGFR5.

Unlike other gene therapy techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The FGFR5 polynucleotide construct can be delivered to the interstitial space of tissues within the an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular, fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked acid sequence injection, an effective dosage amount of DNA or RNA will be in the range of from about 0.05 mg/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and may depend on the condition being treated and the route of administration.

The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked FGFR5 DNA constructs can be delivered to arteries during angioplasty by the catheter used in the procedure.

The naked polynucleotides are delivered by any method known in the art, including, but not limited to, direct needle injection at the delivery site, intravenous injection, topical administration, catheter infusion, and so-called “gene guns”. These delivery methods are known in the art.

As is evidenced in the Examples, naked FGFR5 nucleic acid sequences can be administered in vivo results in the successful expression of FGFR5 polypeptide in the femoral arteries of rabbits.

The constructs may also be delivered with delivery vehicles such as viral sequences, viral particles, liposome formulations, lipofectin, precipitating agents, etc. Such methods of delivery are known in the art.

In certain embodiments, the FGFR5 polynucleotide constructs are complexed in a liposome preparation. Liposomal preparations for use in the instant invention include cationic (positively charged), anionic (negatively charged) and neutral preparations. However, cationic liposomes are particularly preferred because a tight charge complex can be formed between the cationic liposome and the polyanionic nucleic acid. Cationic liposomes have been shown to mediate intracellular delivery of plasmid DNA (Felgner et al., Proc. Natl. Acad. Sci. USA (1987) 84:7413-7416, which is herein incorporated by reference); mRNA (Malone et al., Proc. Natl. Acad. Sci. USA (1989) 86:6077-6081, which is herein incorporated by reference); and purified transcription factors (Debs et al., J. Biol. Chem. (1990) 265:10189-10192, which is herein incorporated by reference), in functional form.

Cationic liposomes are readily available. For example, N[1-2,3-dioleyloxy)propyl]-N,N,N-triethylammonium (DOTMA) liposomes are particularly useful and are available under the trademark Lipofectin, from GIBCO BRL, Grand Island, N.Y. (See, also, Felgner et al., Proc. Natl Acad. Sci. USA (1987) 84:7413-7416, which is herein incorporated by reference). Other commercially available liposomes include transfectace (DDAB/DOPE) and DOTAP/DOPE (Boehringer).

Other cationic liposomes can be prepared from readily available materials using techniques well known in the art. See, e.g. PCT Publication No. WO 90/11092 (which is herein incorporated by reference) for a description of the synthesis of DOTAP (1,2-bis(oleoyloxy)-3-(trimethylammonio)propane) liposomes. Preparation of DOTMA liposomes is explained in the literature, see, e.g., P. Felgner et al., Proc. Natl. Acad. Sci. USA 84:7413-7417, which is herein incorporated by reference. Similar methods can be used to prepare liposomes from other cationic lipid materials.

Similarly, anionic and neutral liposomes are readily available, such as from Avanti Polar Lipids (Birmingham, Ala.), or can be easily prepared using readily available materials. Such materials include phosphatidyl, choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

For example, commercially dioleoylphosphatidyl choline (DOPC), dioleoylphosphatidyl glycerol (DOPG), and dioleoylphosphatidyl ethanolamine (DOPE) can be used in various combinations to make conventional liposomes, with or without the addition of cholesterol. Thus, for example, DOPG/DOPC vesicles can be prepared by drying 50 mg each of DOPG and DOPC under a stream of nitrogen gas into a sonication vial. The sample is placed under a vacuum pump overnight and is hydrated the following day with deionized water. The sample is then sonicated for 2 hours in a capped vial, using a Heat Systems model 350 sonicator equipped with an inverted cup (bath type) probe at the maximum setting while the bath is circulated at 15EC. Alternatively, negatively charged vesicles can be prepared without sonication to produce multilamellar vesicles or by extrusion through nucleopore membranes to produce unilamellar vesicles of discrete size. Other methods are known and available to those of skill in the art.

The liposomes can comprise multilamellar vesicles (MLVs), small unilamellar vesicles (SUVs), or large unilamellar vesicles (LUVs), with SUVs being preferred. The various liposome-nucleic acid complexes are prepared using methods well known in the art. See, e.g., Straubinger et al., Methods of Immunology (1983), 101:512-527, which is herein incorporated by reference. For example, MLVs containing nucleic acid can be prepared by depositing a thin film of phospholipid on the walls of a glass tube and subsequently hydrating with a solution of the material to be encapsulated. SUVs are prepared by extended sonication of MLVs to produce a homogeneous population of unilamellar liposomes. The material to be entrapped is added to a suspension of preformed MLVs and then sonicated. When using liposomes containing cationic lipids, the dried lipid film is resuspended in an appropriate solution such as sterile water or an isotonic buffer solution such as 10 mM Tris/NaCl, sonicated, and then the preformed liposomes are mixed directly with the DNA. The liposome and DNA form a very stable complex due to binding of the positively charged liposomes to the cationic DNA. SUVs find use with small nucleic acid fragments. LUVs are prepared by a number of methods, well known in the art. Commonly used methods include Ca²⁺-EDTA chelation (Papahadjopoulos et al., Biochim. Biophys. Acta (1975) 394:483; Wilson et al., Cell (1979) 17:77); ether injection (Deamer, D. and Bangham, A., Biochim. Biophys. Acta (1976) 443:629; Ostro et al., Biochem. Biophys. Res. Commun. (1977) 76:836; Fraley et al., Proc. Natl. Acad. Sci. USA (1979) 76:3348); detergent dialysis (Enoch, H. and Strittmatter, P., Proc. Natl. Acad. Sci. USA (1979) 76:145); and reverse-phase evaporation (REV) (Fraley et al., J. Biol. Chem. (1980) 255:10431; Szoka, F. and Papahadjopoulos, D., Proc. Natl. Acad. Sci. USA (1978) 75:145; Schaefer-Ridder et al., Science (1982) 215:166), which are herein incorporated by reference.

Generally, the ratio of DNA to liposomes will be from about 10:1 to about 1:10. Preferably, the ration will be from about 5:1 to about 1:5. More preferably, the ration will be about 3:1 to about 1:3. Still more preferably, the ratio will be about 1:1.

U.S. Pat. No. 5,676,954 (which is herein incorporated by reference) reports on the injection of genetic material, complexed with cationic liposomes carriers, into mice. U.S. Pat. Nos. 4,897,355, 4,946,787, 5,049,386, 5,459,127, 5,589,466, 5,693,622, 5,580,859, 5,703,055, and international publication no. WO 94/9469 (which are herein incorporated by reference) provide cationic lipids for use in transfecting DNA into cells and mammals. U.S. Pat. Nos. 5,589,466, 5,693,622, 5,580,859, 5,703,055, and international publication no. WO 94/9469 (which are herein incorporated by reference) provide methods for delivering DNA-cationic lipid complexes to mammals.

In certain embodiments, cells are be engineered, ex vivo or in vivo, using a retroviral particle containing RNA which comprises a sequence encoding FGFR5. Retroviruses from which the retroviral plasmid vectors may be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus.

The retroviral plasmid vector is employed to transduce packaging cell lines to form producer cell lines. Examples of packaging cells which may be transfected include, but are not limited to, the PE501, PA317, R-2, R-AM, PA12, T19-14X, VT-19-17-H2, RCRE, RCRIP, GP+E-86, GP+envAm12, and DAN cell lines as described in Miller, Human Gene Therapy 1:5-14 (1990), which is incorporated herein by reference in its entirety. The vector may transduce the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host.

The producer cell line generates infectious retroviral vector particles which include polynucleotide encoding FGFR5. Such retroviral vector particles then may be employed, to transduce eukaryotic cells, either in vitro or in vivo. The transduced eukaryotic cells will express FGFR5.

In certain other embodiments, cells are engineered, ex vivo or in vivo, with FGFR5 polynucleotide contained in an adenovirus vector. Adenovirus can be manipulated such that it encodes and expresses FGFR5, and at the same time is inactivated in terms of its ability to replicate in a normal lytic viral life cycle. Adenovirus expression is achieved without integration of the viral DNA into the host cell chromosome, thereby alleviating concerns about insertional mutagenesis. Furthermore, adenoviruses have been used as live enteric vaccines for many years with an excellent safety profile (Schwartz, A. R. et al. (1974) Am. Rev. Respir. Dis. 109:233-238). Finally, adenovirus mediated gene transfer has been demonstrated in a number of instances including transfer of alpha-1-antitrypsin and CFTR to the lungs of cotton rats (Rosenfeld, M. A. et al. (1991) Science 252:431-434; Rosenfeld et al., (1992) Cell 68:143-155). Furthermore, extensive studies to attempt to establish adenovirus as a causative agent in human cancer were uniformly negative (Green, M. et al. (1979) Proc. Natl. Acad. Sci. USA 76:6606).

Suitable adenoviral vectors useful in the present invention are described, for example, in Kozarsky and Wilson, Curr. Opin. Genet. Devel. 3:499-503 (1993); Rosenfeld et al., Cell 68:143-155 (1992); Engelhardt et al., Human Genet. Ther. 4:759-769 (1993); Yang et al., Nature Genet. 7:362-369 (1994); Wilson et al., Nature 365:691-692 (1993); and U.S. Pat. No. 5,652,224, which are herein incorporated by reference. For example, the adenovirus vector Ad2 is useful and can be grown in human 293 cells. These cells contain the E1 region of adenovirus and constitutively express Ela and Elb, which complement the defective adenoviruses by providing the products of the genes deleted from the vector. In addition to Ad2, other varieties of adenovirus (e.g., Ad3, Ad5, and Ad7) are also useful in the-present invention.

Preferably, the adenoviruses used in the present invention are replication deficient. Replication deficient adenoviruses require the aid of a helper virus and/or packaging cell line to form infectious particles. The resulting virus is capable of infecting cells and can express a polynucleotide of interest which is operably linked to a promoter, for example, the HARP promoter of the present invention, but cannot replicate in most cells. Replication deficient adenoviruses may be deleted in one or more of all or a portion of the following genes: E1a, E1b, E3, E4, E2a, or L1 through L5.

In certain other embodiments, the cells are engineered, ex vivo or in vivo, using an adeno-associated virus (AAV). AAVs are naturally occurring defective viruses that require helper viruses to produce infectious particles (Muzyczka, N., Curr. Topics in Microbiol. Immunol. 158:97 (1992)). It is also one of the few viruses that may integrate its DNA into non-dividing cells. Vectors containing as little as 300 base pairs of AAV can be packaged and can integrate, but space for exogenous DNA is limited to about 4.5 kb. Methods for producing and using such AAVs are known in the art. See, for example, U.S. Pat. Nos. 5,139,941, 5,173,414, 5,354,678, 5,436,146, 5,474,935, 5,478,745, and 5,589,377.

For example, an appropriate AAV vector for use in the present invention will include all the sequences necessary for DNA replication, encapsidation, and host-cell integration. The FGFR5 polynucleotide construct is inserted into the AAV vector using standard cloning methods, such as those found in Sambrook et al., Molecular Cloning: A Laboratory Manual, Cold Spring Harbor Press (1989). The recombinant AAV vector is then transfected into packaging cells which are infected with a helper virus, using any standard technique, including lipofection, electroporation, calcium phosphate precipitation, etc. Appropriate helper viruses include adenoviruses, cytomegaloviruses, vaccinia viruses, or herpes viruses. Once the packaging cells are transfected and infected, they will produce infectious AAV viral particles which contain the FGFR5 polynucleotide construct. These viral particles are then used to transduce eukaryotic cells, either ex vivo or in vivo. The transduced cells will contain the FGFR5 polynucleotide construct integrated into its genome, and will express FGFR5.

Another method of gene therapy involves operably associating heterologous control regions and endogenous polynucleotide sequences (e.g. encoding FGFR5) via homologous recombination (see, e.g., U.S. Pat. No. 5,641,670, issued Jun. 24, 1997; International Publication No. WO 96/29411, published Sep. 26, 1996; International Publication No. WO 94/12650, published Aug. 4, 1994; Koller et al., Proc. Natl. Acad. Sci. USA 86:8932-8935 (1989); and Zijlstra et al., Nature 342:435-438 (1989). This method involves the activation of a gene which is present in the target cells, but which is not normally expressed in the cells, or is expressed at a lower level than desired.

Polynucleotide constructs are made, using standard techniques known in the art, which contain the promoter with targeting sequences flanking the promoter. Suitable promoters are described herein. The targeting sequence is sufficiently complementary to an endogenous sequence to permit homologous recombination of the promoter-targeting sequence with the endogenous sequence. The targeting sequence will be sufficiently near the 5′ end of the FGFR5 desired endogenous polynucleotide sequence so the promoter will be operably linked to the endogenous sequence upon homologous recombination.

The promoter and the targeting sequences can be amplified using PCR. Preferably, the amplified promoter contains distinct restriction enzyme sites on the 5′ and 3′ ends. Preferably, the 3′ end of the first targeting sequence contains the same restriction enzyme site as the 5′ end of the amplified promoter and the 5′ end of the second targeting sequence contains the same restriction site as the 3′ end of the amplified promoter. The amplified promoter and targeting sequences are digested and ligated together.

The promoter-targeting sequence construct is delivered to the cells, either as naked polynucleotide, or in conjunction with transfection-facilitating agents, such as liposomes, viral sequences, viral particles, whole viruses, lipofection, precipitating agents, etc., described in more detail above. The P promoter-targeting sequence can be delivered by any method, included direct needle injection, intravenous injection, topical administration, catheter infusion, particle accelerators, etc. The methods are described in more detail below.

The promoter-targeting sequence construct is taken up by cells. Homologous recombination between the construct and the endogenous sequence takes place, such that an endogenous FGFR5 sequence is placed under the control of the promoter. The promoter then drives the expression of the endogenous FGFR5 sequence.

The polynucleotides encoding FGFR5 may be administered along with other polynucleotides encoding other angiongenic proteins. Angiogenic proteins include, but are not limited to, acidic and basic fibroblast growth factors, VEGF-1, epidermal growth factor alpha and beta, platelet-derived endothelial cell growth factor, platelet-derived growth factor, tumor necrosis factor alpha, hepatocyte growth factor, insulin like growth factor, colony stimulating factor, macrophage colony stimulating factor, granulocyte/macrophage colony stimulating factor, and nitric oxide synthase.

Preferably, the polynucleotide encoding FGFR5 contains a secretory signal sequence that facilitates secretion of the protein. Typically, the signal sequence is positioned in the coding region of the polynucleotide to be expressed towards or at the 5′ end of the coding region. The signal sequence may be homologous or heterologous to the polynucleotide of interest and may be homologous or heterologous to the cells to be transfected. Additionally, the signal sequence may be chemically synthesized using methods known in the art.

Any mode of administration of any of the above-described polynucleotides constructs can be used so long as the mode results in the expression of one or more molecules in an amount sufficient to provide a therapeutic effect. This includes direct needle injection, systemic injection, catheter infusion, biolistic injectors, particle accelerators (i.e., “gene guns”), gelfoam sponge depots, other commercially available depot materials, osmotic pumps (e.g., Alza minipumps), oral or suppositorial solid (tablet or pill) pharmaceutical formulations, and decanting or topical applications during surgery. For example, direct injection of naked calcium phosphate-precipitated plasmid into rat liver and rat spleen or a protein-coated plasmid into the portal vein has resulted in gene expression of the foreign gene in the rat livers (Kaneda et al., Science 243:375 (1989)).

A preferred method of local administration is by direct injection. Preferably, a recombinant molecule of the present invention complexed with a delivery vehicle is administered by direct injection into or locally within the area of arteries. Administration of a composition locally within the area of arteries refers to injecting the composition centimeters and preferably, millimeters within arteries.

Another method of local administration is to contact a polynucleotide construct of the present invention in or around a surgical wound. For example, a patient can undergo surgery and the polynucleotide construct can be coated on the surface of tissue inside the wound or the construct can be injected into areas of tissue inside the wound.

Therapeutic compositions useful in systemic administration, include recombinant molecules of the present invention complexed to a targeted delivery vehicle of the present invention. Suitable delivery vehicles for use with systemic administration comprise liposomes comprising ligands for targeting the vehicle to a particular site.

Preferred methods of systemic administration, include intravenous injection, aerosol, oral and percutaneous (topical) delivery. Intravenous injections can be performed using methods standard in the art. Aerosol delivery can also be performed using methods standard in the art (see, for example, Stribling et al., Proc. Natl. Acad. Sci. USA 189:11277-11281, 1992, which is incorporated herein by reference). Oral delivery can be performed by complexing a polynucleotide construct of the present invention to a carrier capable of withstanding degradation by digestive enzymes in the gut of an animal. Examples of such carriers, include plastic capsules or tablets, such as those known in the art. Topical delivery can be performed by mixing a polynucleotide construct of the present invention with a lipophilic reagent (e.g., DMSO) that is capable of passing into the skin.

Determining an effective amount of substance to be delivered can depend upon a number of factors including, for example, the chemical structure and biological activity of the substance, the age and weight of the animal, the precise condition requiring treatment and its severity, and the route of administration. The frequency of treatments depends upon a number of factors, such as the amount of polynucleotide constructs administered per dose, as well as the health and history of the subject. The precise amount, number of doses, and timing of doses will be determined by the attending physician or veterinarian. Therapeutic compositions of the present invention can be administered to any animal, preferably to mammals and birds. Preferred mammals include humans, dogs, cats, mice, rats, rabbits sheep, cattle, horses and pigs, with humans being particularly preferred.

### Prophylactic and Therapeutic Methods

It is to be understood that although the following discussion is specifically directed to human patients, the teachings are also applicable to any animal that expresses FGFR5.

As noted above, FGFR5 is structurally related to members of the fibroblast growth factor receptor family and is most highly homologous to FGFR4, which has been shown to be involved in the binding of fibroblast growth factors, and mediate the effects of these FGFs in a tissue specific manner. Thus, it is likely that FGFR5 is active in modulating growth regulatory activities (e.g., cell survival, secretion, differentiation and/or cell proliferation) and cellular profileration. Further, FGFR5, like FGFR4, is likely to be involved in the development of skeletal muscle and endodermal cells. Additionally, the expression profile of FGFR5 suggests that it plays a role in a broader variety of cell types than observed for FGFR4. Particularly, FGFR5 is expressed in cardiac tissue in addition to skeletal muscle cells, cells of the pancreas, cells and tissue of the immune system, and cells and tissue of the renal system. Thus FGFR5 plays a role in regulating the binding of fibroblast growth factors in a tissue specific manner, and this binding is likely to result in activation, survival, proliferation, migration, and differentiation, as well as the regulation of the growth of the previously mentioned cells, as well as cells from other tissues. Thus FGFR5 is likely to play a role in influencing various diseases or medical conditions, including, but not limited to, mucositis; wound healing, angiogenesis, immune function, endocrine function, and insulin secretion. Additionally, FGFR5 appears to be expressed in other cell populations (e.g., brain cells and kidney cells) and thus FGFR5 likely plays a role in the regulation and/or proliferation of these cells, thereby regulating their survival, differentiation, and morphology. Accordingly, it is likely that FGFR5 plays a role in other physiological or disease conditions, including, cancer, angiogenesis, wound healing, fibrosis, and tissue regeneration.

Any method which neutralizes or enhances FGFR5 mediated signaling can be used to modulate growth regulatory activities (e.g., cell proliferation), and other activities mediated by FGFR5 signaling, such as, for example, tissue vascularization, cellular proliferation, host defense, inflammation, immune surveillance, arthritis, mucositis, immune dysfunction, cancer, angiogenesis, wound healing, organ rejection, fibrosis, and tissue regeneration.

FGFR5 polynucleotides or polypeptides (including FGFR5 fragments, variants, derivatives, and analogs, and FGFR5 agonists and antagonists as described herein) are useful in treating disorders associated with wound healing, angiogenesis, inflammation, mucositis, immune function, endocrine function, insulin secretion, and ischemia. Additionally, these compounds may be useful in treating or preventing cell death (e.g., of tissues damaged by severe wounds).

FGFR5 polynucleotides or polypeptides (including FGFR5 fragments, variants, derivatives, and analogs, and FGFR5 agonists or FGFR5 antagonists as described herein) are useful in treating or detecting disorders involving the growth of cells, including, for example, endothelial and mesenchymal cells, or disorders involving angiogenesis. FGFR5 polynucleotides or polypeptides, or agonists of these, could be used to stimulate the regrowth of the tissues suffering from insults such as wounds. Alternatively, antagonists of FGFR5 could be used to inhibit cell growth and proliferation of neoplastic cells and tissues, and thereby inhibit or prevent abnormal cellular growth or proliferation, such as those seen with tumors.

FGFR5 polynucleotides or polypeptides (including FGFR5 fragments, variants, derivatives, and analogs, and FGFR5 agonists and antagonists as described herein) are useful to differentiate, proliferate, and attract cells, leading to the regeneration of tissues. (See, Science 276:59-87 (1997). The regeneration of tissues could be used to repair, replace, or protect tissue damaged by congenital defects, trauma (wounds, burns, incisions, or ulcers), age, disease (e.g. osteoporosis, osteocarthritis, periodontal disease, liver failure), surgery, including cosmetic plastic surgery, fibrosis, reperfusion injury, or systemic cytokine damage. Tissues that may be regenerated using the present invention include organs (e.g., pancreas, liver, intestine, kidney, skin, endothelium), muscle (smooth, skeletal or cardiac), vascular (including vascular endothelium), nervous, hematopoietic, and skeletal (bone, cartilage, tendon, and ligament) tissue. Preferably, regeneration occurs without or decreased scarring. Regeneration also may include angiogenesis. Additionally, these compounds can be used to treat or prevent cell death (e.g., hematopoietic cell death) during processes of inflammation or tissue injury.

Moreover, FGFR5 polypeptides or polynucleotides (including FGFR5 fragments, variants, derivatives, and analogs, and FGFR5 agonists and antagonists as described herein) are useful to modulate mucositis (the destruction of the mucosal tissues). Furthermore, by increasing fibroblast growth factor activity, FGFR5 polynucleotides or polypeptides could be used to stimulate the proliferation, or prolong the life of, mucosal tissues, and thus reduce or eliminate the effects of mucositis brought on by any number of agents, such as, for example, chemotherapy agents.

FGFR5 polynucleotides or polypeptides (including FGFR5 fragments, variants, derivatives, and analogs, and FGFR5 agonists or FGFR5 antagonists as described herein) are useful in treating or detecting disorders involving the growth and vascularization of endothelial tissues. FGFR5 polynucleotides or polypeptides, or agonists of these, could be used to stimulate the revascularization of ischemic tissues damaged from conditions such as thrombosis, arteriosclerosis, and other cardiovascular conditions. Antagonists of FGFR5 polynucleotides or polypeptides could be used to prevent hyper-vascular diseases.

FGFR5 polypeptides or polynucleotides (including FGFR5 fragments, variants, derivatives, and analogs, and FGFR5 agonists and antagonists as described herein) are useful to treat or detect hyperproliferative disorders, including neoplasms. FGFR5 polypeptides or polynucleotides and/or FGFR5 agonists or antagonists, may inhibit the proliferation of the disorder through direct or indirect interactions. Alternatively, FGFR5 polypeptides or polynucleotides and/or FGFR5 agonists or antagonists may proliferate other cells which can inhibit the hyperproliferative disorder. For example, by increasing an immune response, particularly increasing antigenic qualities of the hyperproliferative disorder or by proliferating, differentiating, or mobilizing T-cells, hyperproliferative disorders can be treated. This immune response may be increased by either enhancing an existing immune response, or by initiating a new immune response. Alternatively, decreasing an immune response may also be a method of treating hyperproliferative disorders, such as a chemotherapeutic agent.

Examples of hyperproliferative disorders that can be treated or detected by FGFR5 polynucleotides or polypeptides and/or FGFR5 agonists or antagonists include, but are not limited to, neoplasms located in the: pancreas, kidney, skeletal muscle, heart, liver, brain, skin, soft tissue, blood, abdomen, bone, lung, digestive system, breast, prostate, peritoneum, endocrine glands (adrenal, parathyroid, pituitary, testicles, ovary, thymus, thyroid), eye, head and neck, nervous (central and peripheral), lymphatic system, hematopoietic tissue, pelvic, spleen, thoracic, and urogenital.

Similarly, other hyperproliferative disorders can also be treated or detected by FGFR5 polynucleotides or polypeptides and/or FGFR5 agonists or antagonists. Examples of such hyperproliferative disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

FGFR5 polynucleotides or polypeptides (including FGFR5 fragments, variants, derivatives, and analogs, and FGFR5 agonists and FGFR5 antagonists as described herein) are useful to treat and/or prevent organ rejection or graft-versus-host disease (GVHD). Organ rejection occurs by host immune cell destruction of the transplanted tissue through an immune response. Similarly, an immune response is also involved in GVHD, but, in this case, the foreign transplanted immune cells destroy the host tissues. The administration of FGFR5 polypeptides or polynucleotides and/or FGFR5 agonists or antagonists that inhibits an immune response, particularly the proliferation, differentiation, or chemotaxis of T-cells, may be an effective therapy in preventing organ rejection or GVHD.

FGFR5 polypeptides or polynucleotides (including FGFR5 fragments, variants, derivatives, and analogs, and FGFR5 agonists and antagonists as described herein) are useful in treating deficiencies or disorders of the immune system, by activating or inhibiting the proliferation, differentiation, or mobilization (chemotaxis) of immune cells. Immune cells develop through a process called hematopoiesis, producing myeloid (platelets, red blood cells, neutrophils, and macrophages) and lymphoid (B and T lymphocytes) cells from pluripotent stem cells. The etiology of these immune deficiencies or disorders may be genetic, somatic, such as cancer or some autoimmune disorders, acquired (e.g., by chemotherapy or toxins), or infectious. Moreover, FGFR5 polynucleotides or polypeptides can be used as a marker or detector of a particular immune system disease or disorder.

Similarly, FGFR5 polypeptides or polynucleotides (including FGFR5 fragments, variants, derivatives, and analogs, and FGFR5 agonists and FGFR5 antagonists as described herein) are useful to modulate inflammation. For example, FGFR5 polypeptides or polynucleotides and/or FGFR5 agonists and antagonists of the invention may inhibit the proliferation and differentiation of cells involved in an inflammatory response or alternatively may be involved in killing of hematopoietic cells during processes of inflammation or tissue injury. These molecules can be used to treat inflammatory conditions, both chronic and acute conditions, including ischemia-reperfusion injury, arthritis, and/or nephritis. Additionally, these molecules may be used to treat or prevent killing of hematopoietic cells and/or other cells during processes of inflammation or tissue injury.

FGFR5 polypeptides or polynucleotides (including FGFR5 fragments, variants, derivatives, and analogs, and FGFR5 agonists and antagonists as described herein) are useful to treat or detect infectious agents. For example, by increasing the immune response, particularly increasing the proliferation and differentiation of B and/or T cells, infectious diseases may be treated. The immune response may be increased by either enhancing an existing immune response, or by initiating a new immune response. Alternatively, FGFR5 polypeptides or polynucleotides and/or FGFR5 agonists or antagonists may also directly inhibit the infectious agent, without necessarily eliciting an immune response.

Moreover, FGFR5 polynucleotides or polypeptides (including FGFR5 fragments, variants, derivatives, and analogs, and FGFR5 agonists and antagonists as described herein) are useful to increase regeneration of tissues difficult to heal. For example, increased tendon/ligament regeneration would quicken recovery time after damage. FGFR5 polynucleotides or polypeptides and/or FGFR5 agonists or antagonists of the present invention could also be used prophylactically in an effort to avoid damage. Specific diseases that could be treated include tendinitis, carpal tunnel syndrome, and other tendon or ligament defects. A further example of tissue regeneration of non-healing wounds includes pressure ulcers, ulcers associated with vascular insufficiency, surgical, and traumatic wounds.

Given the activities modulated by FGFR5, it is readily apparent that a substantially altered (increased or decreased) level of expression of FGFR5 in an individual compared to the standard or “normal” level produces pathological conditions such as those described above. It will also be appreciated by one of ordinary skill that the FGFR5 agonists of the invention will exert modulating activities on any of its target cells. Therefore, it will be appreciated that conditions caused by a decrease in the standard or normal level of FGFR5 mediated activity in an individual, can be treated by administration of FGFR5 polypeptide or an agonist thereof.

Thus, in one embodiment, the present invention is directed to a method for enhancing (i.e., increasing) FGFR5 mediated activity (e.g., cellular growth and proliferation) which involves administering to an individual in need of an increased level of FGFR5 mediated activity, a therapeutically effective amount of FGFR5 polypeptide, fragment, variant, derivative, or analog, or an agonist capable of increasing FGFR5 mediated activity. In specific embodiments, FGFR5 mediated signaling is increased to treat a disease or condition wherein decreased cell survival, secretion, proliferation, migration, and/or differentiation is exhibited.

In another embodiment, the present invention is directed to a method for suppressing (i.e., decreasing) FGFR5 mediated activity (e.g., cellular growth and proliferation), which involves administering to an individual in need of a decreased level of FGFR5 mediated activity, a therapeutically effective amount of FGFR5 polypeptide, fragment, variant, derivative, or analog or an antagonist capable of decreasing FGFR5 mediated activity. In specific embodiments, FGFR5 mediated signaling is decreased to treat a disease or condition wherein increased cell survival, secretion, proliferation, migration and/or differentiation is exhibited.

In addition to treating diseases associated with elevated or decreased levels of FGFR5 mediated activity, the invention encompasses methods of administering FGFR5 agonists or antagonists to elevate or reduce FGFR5 mediated biological activity, respectively.

For treating abnormal conditions related to an under-expression of FGFR5 and its activity, or in which elevated or decreased levels of FGFR5 are desired, several approaches are available. One approach comprises administering to an individual in need of an increased level of FGFR5 mediated activity in the body, a therapeutically effective amount of an isolated FGFR5 polypeptide, fragment, variant, derivative or analog of the invention, or a compound which activates FGFR5, i.e., an agonist as described above, optionally in combination with a pharmaceutically acceptable carrier. Alternatively, gene therapy may be employed to effect the endogenous production of FGFR5 by the relevant cells in the subject. For example, a polynucleotide of the invention may be engineered for expression in a replication defective retroviral vector using techniques known in the art. The retroviral expression construct may then be isolated and introduced into a packaging cell transduced with a retroviral plasmid vector containing RNA encoding a polypeptide of the present invention such that the packaging cell now produces infectious viral particles containing the gene of interest. These producer cells may be administered to a subject for engineering cells in vivo and expression of the polypeptide in vivo. For an overview of gene therapy, see Chapter 20, Gene Therapy and other Molecular Genetic-based Therapeutic Approaches, (and references cited therein) in Human Molecular Genetics, T Strachan and A P Read, BIOS Scientific Publishers Ltd (1996).

Further, treatment can be administered, for example, in the form of gene replacement therapy. Specifically, one or more copies of a FGFR5 nucleotide sequence of the invention that directs the production of a FGFR5 gene product exhibiting normal function, may be inserted into the appropriate cells within a patient or animal subject, using vectors which include, but are not limited to, adenovirus, adeno-associated virus, retrovirus and herpesvirus vectors, in addition to other particles that introduce DNA into cells, such as liposomes and gene activated matrices. Because the FGFR5 gene is expressed in hematopoietic tissue, including liver/fetal liver, etc, such gene replacement techniques should be capable of delivering FGFR5 gene sequence to these cells within patients, or, alternatively, should involve direct administration of such FGFR5 polynucleotide sequences to the site of the cells in which the FGFR5 gene sequences are to be expressed. Alternatively, targeted homologous recombination can be utilized to correct the defective endogenous FGFR5 gene and/or regulatory sequences thereof (e.g., promoter and enhancer sequences), or alternatively, to “turn on” other dormant FGFR5 activity in the appropriate tissue or cell type.

Additional methods which may be utilized to increase the overall level of FGFR5 expression and/or FGFR5 activity include the introduction of appropriate FGFR5-expressing cells, preferably autologous cells, into a patient at positions and in numbers which are sufficient to ameliorate the symptoms of abnormalities in cell growth regulation, cell signaling, and other FGFR5 mediated activities. Such cells may be either recombinant or non-recombinant. Among the cells which can be administered to increase the overall level of FGFR5 gene expression in a patient are normal cells, which express the FGFR5 gene. Cell-based gene therapy techniques are well known to those skilled in the art, see, e.g., Anderson et al., U.S. Pat. No. 5,399,349; and Mulligan & Wilson, U.S. Pat. No. 5,460,959.

Thus, one embodiment of the invention comprises administering to in individual in need of an increased level of FGFR5 mediated activity compound that stimulates FGFR5 mediated activity (agonist), such as for example, an antibody or FGFR5 fragment, variant, derivative or analog of the invention, along with a pharmaceutically acceptable carrier in an amount effective to enhance (i.e., increase) FGFR5 mediated activity.

If the activity of FGFR5 is in excess, several approaches are available to reduce or inhibit FGFR5 activity using molecules derived from the polypeptide and polynucleotide sequences described above. Accordingly, a further aspect of the invention is related to a method for treating an individual in need of a decreased level of FGFR5 mediated activity in the body comprising, administering to such an individual a composition comprising a therapeutically effective amount of a FGFR5 polypeptide, fragment, variant, derivative or analog of the invention which acts as a FGFR5 antagonist or FGFR5 antagonist identified using the methods described herein, optionally, in combination with a pharmaceutically acceptable carrier. Preferably, FGFR5 activity is decreased to treat a disease wherein increased cell survival, secretion, proliferation, migration, and/or differentiation is exhibited. Polypeptides, derivatives, variants and analogs of the invention and other compounds which function as antagonists of FGFR5 can routinely be identified using the assays described infra and other techniques known in the art. Preferred antagonists for use in the present invention are FGFR5-specific antibodies.

In another approach, FGFR5 activity can be reduced or inhibited by decreasing the level of FGFR5 gene expression. In one embodiment, this is accomplished through the use of antisense sequences, either internally generated or separately administered (see, for example, O'Connor, J. Neurochem. (1991) 56:560 in Oligodeoxynucleotides as Antisense Inhibitors of Gene Expression, CRC Press, Boca Raton, Fla. (1988). Antisense technology can be used to control gene expression through antisense DNA or RNA or through triple-helix formation. Antisense techniques are discussed, for example, in Okano, J. Neurochem. 56:560 (1991); Oligodeoxynucleotides as Antisense Inhibitors of Gene Expression, CRC Press, Boca Raton, Fla. (1988). Triple helix formation is discussed in, for instance, Lee et al., Nucleic Acids Research 6:3073 (1979); Cooney et al., Science 241:456 (1988); and Dervan et al., Science 251:1360 (1991). The methods are based on binding of a polynucleotide to a complementary DNA or RNA. For example, the 5′ coding portion of a polynucleotide that encodes FGFR5 polypeptide of the present invention may be used to design an antisense RNA oligonucleotide of from about 10 to 40 base pairs in length. A DNA oligonucleotide is designed to be complementary to a region of the gene involved in transcription thereby preventing transcription and the production of the FGFR5 polypeptide. The antisense RNA oligonucleotide hybridizes to the mRNA in vivo and blocks translation of the mRNA molecule into polypeptide.

In one embodiment, the FGFR5 antisense nucleic acid of the invention is produced intracellularly by transcription from an exogenous sequence. For example, a vector or a portion thereof, is transcribed, producing an antisense nucleic acid (RNA) of the invention. Such a vector would contain a sequence encoding the FGFR5 antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others know in the art, used for replication and expression in vertebrate cells. Expression of the sequence encoding FGFR5, or fragments thereof, can be by any promoter known in the art to act in vertebrate, preferably human cells. Such promoters can be inducible or constitutive. Such promoters include, but are not limited to, the SV40 early promoter region (Bernoist and Chambon, Nature 29:304-310 (1981), the promoter contained in the 3′ long terminal repeat of Rous sarcoma virus (Yamamoto et al., Cell 22:787-797 (1980)), the herpes thymidine promoter (Wagner et al., Proc. Natl. Acad. Sci. U.S.A. 78:1441-1445 (1981)), the regulatory sequences of the metallothionein gene (Brinster et al., Nature 296:39-42 (1982)), etc.

The antisense nucleic acids of the invention comprise a sequence complementary to at least a portion of an RNA transcript of a FGFR5 gene. However, absolute complementarity, although preferred, is not required. A sequence “complementary to at least a portion of an RNA,” referred to herein, means a sequence having sufficient complementarity to be able to hybridize with the RNA, forming a stable duplex; in the case of double stranded FGFR5 antisense nucleic acids, a single strand of the duplex DNA may thus be tested, or triplex formation may be assayed. The ability to hybridize will depend on both the degree of complementarity and the length of the antisense nucleic acid. Generally, the larger the hybridizing nucleic acid, the more base mismatches with a FGFR5 RNA it may contain and still form a stable duplex (or triplex as the case may be). One skilled in the art can ascertain a tolerable degree of mismatch by use of standard procedures to determine the melting point of the hybridized complex.

Potential FGFR5 antagonists according to the invention also include catalytic RNA, or a ribozyme (See, e.g., PCT International Publication WO 90/11364, published Oct. 4, 1990; Sarver et al., Science 247:1222-1225 (1990). While ribozymes that cleave mRNA at site specific recognition sequences can be used to destroy FGFR5 mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, Nature 334:585-591 (1988). There are numerous potential hammerhead ribozyme cleavage sites within the nucleotide sequence of FGFR5 (FIGS. 1A-C; SEQ ID NO:1). Preferably, the ribozyme is engineered so that the cleavage recognition site is located near the 5′ end of the FGFR5 mRNA; i.e., to increase efficiency and minimize the intracellular accumulation of non-functional mRNA transcripts. DNA constructs encoding the ribozyme may be introduced into the cell in the same manner as described above for the introduction of antisense encoding DNA. Since ribozymes, unlike antisense molecules are catalytic, a lower intracellular concentration is required for efficiency.

Endogenous FGFR5 gene expression can also be reduced by inactivating or “knocking out” the FGFR5 gene or its promoter using targeted homologous recombination (e.g., see Smithies et al., Nature 317:330-234 (1985); Thomas et al., Cell 51:503-512 (1987); Thompson et al., Cell 5:313-321(1989); each of which is incorporated by reference herein in its entirety). Such approach can be adapted for use in humans provided the recombinant DNA constructs are directly administered or targeted to the required site in vivo using appropriate viral vectors.

Alternatively, endogenous FGFR5 gene expression can be reduced by targeted deoxyribonucleotide sequences complementary to the regulatory region of the FGFR5 gene (i.e., the FGFR5 promoter and/or enhancers) to form triple helical structures that prevent transcription of the FGFR5 gene in target cells in the body, see generally, Helene et al., Ann, N.Y. Acad. Sci. 660:27-36 (1992); Helene, C., Anticancer Drug Des., 6(6):569-584 (1991); and Maher, L. J., Bioassays 14(12):807-815 (1992)).

Thus, one embodiment of the invention comprises administering to an individual in need of a decreased level of FGFR5 mediated activity, a FGFR5 inhibitor compound (antagonist), such as for example, an antibody or FGFR5 fragment, variant, derivative or analog of the invention, along with a pharmaceutically acceptable carrier in an amount effective to suppress (i.e., lower) FGFR5 mediated activity.

### Transgenics/“Knock-outs”

The proteins of the invention can also be expressed in transgenic animals. Animals of any species, including, but not limited to, mice, rats, rabbits, hamsters, guinea pigs, pigs, micro-pigs, goats, sheep, cows and non-human primates, e.g., baboons, monkeys, and chimpanzees may be used to generate transgenic animals. In a specific embodiment, techniques described herein or otherwise known in the art, are used to express polypeptides of the invention in humans, as part of a gene therapy protocol.

Any technique known in the art may be used to introduce the transgene (i.e., nucleic acids of the invention) into animals to produce the founder lines of transgenic animals. Such techniques include, but are not limited to, pronuclear microinjection (Paterson et al., Appl. Microbiol. Biotechnol. 40:691-698 (1994); Carver et al., Biotechnology (NY) 11: 1263-1270 (1993); Wright et al., Biotechnology (NY) 9:830-834 (1991); and Hoppe et al., U.S. Pat. No. 4,873,191-(1989)); retrovirus mediated gene transfer into germ lines (Van der Putten et al., Proc. Natl. Acad. Sci., USA 82:6148-6152 (1985)), blastocysts or embryos; gene targeting in embryonic stem cells (Thompson et al., Cell 56:313-321 (1989)); electroporation of cells or embryos (Lo, Mol Cell. Biol. 3:1803-1814 (1983)); introduction of the polynucleotides of the invention using a gene gun (see, e.g., Ulmer et al., Science 259:1745 (1993); introducing nucleic acid constructs into embryonic pleuripotent stem cells and transferring the stem cells back into the blastocyst, and sperm-mediated gene transfer (Lavitrano et al., Cell 57:717-723 (1989); etc. For a review of such techniques, see Gordon, “Transgenic Animals,” Intl. Rev. Cytol. 115:171-229 (1989), which is incorporated by reference herein in its entirety. Further, the contents of each of the documents recited in this paragraph is herein incorporated by reference in its entirety.

Any technique known in the art may be used to produce transgenic clones containing polynucleotides of the invention, for example, nuclear transfer into enucleated oocytes of nuclei from cultured embryonic, fetal, or adult cells induced to quiescence (Campell et al., Nature 380:64-66 (1996); Wilmut et al., Nature 385:810-813 (1997)), each of which is herein incorporated by reference in its entirety).

The present invention provides for transgenic animals that carry the transgene in all their cells, as well as animals which carry the transgene in some, but not all their cells, i.e., mosaic animals or chimeric animals. The transgene may be integrated as a single transgene or as multiple copies such as in concatamers, e.g., head-to-head tandems or head-to-tail tandems. The transgene may also be selectively introduced into and activated in a particular cell type by following, for example, the teaching of Lasko et al. (Lasko et al., Proc. Natl. Acad. Sci. USA 89:6232-6236 (1992)). The regulatory sequences required for such a cell-type specific activation will depend upon the particular cell type of interest, and will be apparent to those of skill in the art. When it is desired that the polynucleotide transgene be integrated into the chromosomal site of the endogenous gene, gene targeting is preferred. Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene. The transgene may also be selectively introduced into a particular cell type, thus inactivating the endogenous gene in only that cell type, by following, for example, the teaching of Gu et al. (Gu et al., Science 265:103-106 (1994)). The regulatory sequences required for such a cell-type specific inactivation will depend upon the particular cell type of interest, and will be apparent to those of skill in the art. The contents of each of the documents recited in this paragraph is herein incorporated by reference in its entirety.

Once transgenic animals have been generated, the expression of the recombinant gene may be assayed utilizing standard techniques. Initial screening may be accomplished by Southern blot analysis or PCR techniques to analyze animal tissues to verify that integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and reverse transcriptase-PCR (rt-PCR). Samples of transgenic gene-expressing tissue may also be evaluated immunocytochemically or immunohistochemically using antibodies specific for the transgene product.

Once the founder animals are produced, they may be bred, inbred, outbred, or crossbred to produce colonies of the particular animal. Examples of such breeding strategies include, but are not limited to: outbreeding of founder animals with more than one integration site in order to establish separate lines; inbreeding of separate lines in order to produce compound transgenics that express the transgene at higher levels because of the effects of additive expression of each transgene; crossing of heterozygous transgenic animals to produce animals homozygous for a given integration site in order to both augment expression and eliminate the need for screening of animals by DNA analysis; crossing of separate homozygous lines to produce compound heterozygous or homozygous lines; and breeding to place the transgene on a distinct background that is appropriate for an experimental model of interest.

Transgenic and “knock-out” animals of the invention have uses which include, but are not limited to, animal model systems useful in elaborating the biological function of FGFR5 polypeptides, studying conditions and/or disorders associated with aberrant FGFR5 expression, and in screening for compounds effective in ameliorating such conditions and/or disorders.

In further embodiments of the invention, cells that are genetically engineered to express the proteins of the invention, or alternatively, that are genetically engineered not to express the proteins of the invention (e.g., knockouts) are administered to a patient in vivo. Such cells may be obtained from the patient (i.e., animal, including human) or an MHC compatible donor and can include, but are not limited to fibroblasts, bone marrow cells, blood-cells (e.g., lymphocytes), adipocytes, muscle cells, endothelial cells, etc. The cells are genetically engineered in vitro using recombinant DNA techniques to introduce the coding sequence of polypeptides of the invention into the cells, or alternatively, to disrupt the coding sequence and/or endogenous regulatory sequence associated with the polypeptides of the invention, e.g., by transduction (using viral vectors, and preferably vectors that integrate the transgene into the cell genome) or transfection procedures, including, but not limited to, the use of plasmids, cosmids, YACs, naked DNA, electroporation, liposomes, etc. The coding sequence of the polypeptides of the invention can be placed under the control of a strong constitutive or inducible promoter or promoter/enhancer to achieve expression, and preferably secretion, of the polypeptides of the invention. The engineered cells which express and preferably secrete the polypeptides of the invention can be introduced into the patient systemically, e.g., in the circulation, or intraperitoneally. Alternatively, the cells can be incorporated into a matrix and implanted in the body, e.g., genetically engineered fibroblasts can be implanted as part of a skin graft; genetically engineered endothelial cells can be implanted as part of a lymphatic or vascular graft. (See, for example, Anderson et al. U.S. Pat. No. 5,399,349; and Mulligan & Wilson, U.S. Pat. No. 5,460,959, each of which is incorporated by reference herein in its entirety).

When the cells to be administered are non-autologous or non-MHC compatible cells, they can be administered using well known techniques which prevent the development of a host immune response against the introduced cells. For example, the cells may be introduced in an encapsulated form which, while allowing for an exchange of components with the immediate extracellular environment, does not allow the introduced cells to be recognized by the host immune system.

### Formulation and Administration

It will be appreciated that conditions caused by a decrease in the standard or normal level of FGFR5 mediated activity in an individual, can be treated by administration of FGFR5 polypeptide or fragment, variant, derivative, or analog of the invention or an agonist thereof. Thus, the invention further provides a method of treating an individual in need of an increased level of FGFR5 mediated activity comprising administering to such an individual a pharmaceutical composition comprising an effective amount of an isolated FGFR5 polynucleotide or polypeptide; or fragment, variant, derivative, or analog of the invention, such as for example, the full length form of the FGFR5 encoding polynucleotide, effective to increase the FGFR5 mediated activity level in such an individual.

It will be appreciated that conditions caused by a decrease in the standard or normal level of FGFR5 mediated activity in an individual, can be treated by administration of FGFR5 polypeptide or fragment, variant, derivative, or analog of the invention or an agonist thereof. Thus, the invention further provides a method of treating an individual in need of an increased level of FGFR5 mediated activity comprising administering to such an individual a pharmaceutical composition comprising an effective amount of an isolated FGFR5 polynucleotide or polypeptide; or fragment, variant, derivative, or analog of the invention, such as for example, the full length form of the FGFR5 encoding polynucleotide, effective to increase the FGFR5 mediated activity level in such an individual.

The dosage range required depends on the choice of peptide, the route of administration, the nature of the formulation, the nature of the subject's condition, and the judgment of the attending practitioner. As a general proposition, the total pharmaceutically effective amount of FGFR5 polypeptide administered parenterally per dose will be in the range of about 1 μg/kg/day to 10 mg/kg/day of patient body weight, although, as noted above, this will be subject to therapeutic discretion. More preferably, this dose is at least 0.01 mg/kg/day, and most preferably for humans this dose is in the range of 0.1-100 mg/kg of subject, or between about 0.01 and 1 mg/kg/day. If given continuously, the FGFR5 polypeptide is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

Pharmaceutical compositions containing the FGFR5 polypeptides and polynucleotides of the invention (including fragments, variants, derivatives or analogs), and FGFR5 agonists and antagonists may be routinely formulated in combination with a pharmaceutically acceptable carrier. By “pharmaceutically acceptable carrier” is meant a non-toxic solid, semisolid or liquid filler, diluent, encapsulating material or formulation auxiliary of any type. In a specific embodiment, “pharmaceutically acceptable” means approved by a regulatory agency of the federal or a state government or listed in the U.S. Pharmacopeia or other generally recognized pharmacopeia for use in animals, and more particularly humans. Nonlimiting examples of suitable pharmaceutical carriers according to this embodiment are provided in “Remington's Pharmaceutical Sciences” by E. W. Martin, and include sterile liquids, such as water, saline, buffered saline, glycerol, ethanol, and oils, including those of petroleum, animal, vegetable or synthetic origin, such as peanut oil, soybean oil, mineral oil, sesame oil and the like. Formulation should suit the mode of administration, and is well within the skill of the art. For example, water is a preferred carrier when the pharmaceutical composition is administered intravenously. Saline solutions and aqueous dextrose and glycerol solutions can be employed as liquid carriers, particularly for injectable solutions. The invention additionally relates to pharmaceutical packs and kits comprising one or more containers filled with one or more of the ingredients of the aforementioned compositions of the invention.

Polypeptides and other compounds of the present invention may be administered alone or in conjunction with other compounds, such as therapeutic compounds. The pharmaceutical composition of the invention may be administered orally, rectally, parenterally, intracistemally, intravaginally, intraperitoneally, topically (as by powders, ointments, drops or transdermal patch), bucally, or as an oral or nasal spray. Preferred forms of systemic administration of the pharmaceutical compositions include parenteral injection, typically by intravenous injection. Other injection routes, such as subcutaneous, intramuscular, intrasternal, intraarticular or intraperitoneal, can be used. Alternative means for systemic administration include transmucosal and transdermal administration using penetrants such as bile salts or fusidic acids or other detergents. In addition, if properly formulated in enteric or encapsulated formulations, oral administration may also be possible. Administration of these compounds may also be topical and/or localized, in the form of salves, pastes, gels and the like.

Polypeptides used in treatment can also be generated endogenously in the subject, in treatment modalities often referred to as “gene therapy” as described above. Thus, for example, cells from a subject may be engineered with a polynucleotide, such as a DNA or RNA, to encode a polypeptide ex vivo, and for example, by the use of a retroviral plasmid vector. The cells are then introduced into the subject.

### Gene Mapping

The nucleic acid molecules of the present invention are also valuable for chromosome identification. The sequence is specifically targeted to and can hybridize with a particular location on an individual human chromosome. Moreover, there is a current need for identifying particular sites on the chromosome. Few chromosome marking reagents based on actual sequence data (repeat polymorphisms) are presently available for marking chromosomal location. The mapping of DNAs to chromosomes according to the present invention is an important first step in correlating those sequences with genes associated with disease.

In certain preferred embodiments in this regard, the cDNA herein disclosed is used to clone genomic DNA of a FGFR5 gene. This can be accomplished using a variety of well known techniques and libraries, which generally are available commercially. The genomic DNA then is used for in situ chromosome mapping using well known techniques for this purpose.

In addition, in some cases, sequences can be mapped to chromosomes by preparing PCR primers (preferably 15-25 bp) from the cDNA. Computer analysis of the 3′ untranslated region of the gene is used to rapidly select primers that do not span more than one exon in the genomic DNA, thus complicating the amplification process. These primers are then used for PCR screening of somatic cell hybrids containing individual human chromosomes. Fluorescence in situ hybridization (“FISH”) of a cDNA clone to a metaphase chromosomal spread can be used to provide a precise chromosomal location in one step. This technique can be used with probes from the cDNA as short as 50 or 60 bp. For a review of this technique, see Verma et al., Human Chromosomes: A Manual Of Basic Techniques, Pergamon Press, New York (1988).

Once a sequence has been mapped to a precise chromosomal location, the physical position of the sequence on the chromosome can be correlated with genetic map data. Such data are found, for example, in V. McKusick, Mendelian Inheritance In Man, available on-line through Johns Hopkins University, Welch Medical Library. The relationship between genes and diseases that have been mapped to the same chromosomal region are then identified through linkage analysis (coinheritance of physically adjacent genes).

Next, it is necessary to determine the differences in the cDNA or genomic sequence between affected and unaffected individuals. If a mutation is observed in some or all of the affected individuals but not in any normal individuals, then the mutation is likely to be the causative agent of the disease.

Having generally described the invention, the same will be more readily understood by reference to the following examples, which are provided by way of illustration and are not intended as limiting. Thus, the present invention is not to be limited in scope by the specific embodiments described herein, which are intended as single illustrations of individual aspects of the invention.

### EXAMPLES

### Example 1

### Isolation of the FGFR5 cDNA Clone From the Deposited Sample

The cDNA for FGFR5 is inserted into the EcoRI and Xho I multiple cloning site of pCMVSport 3.0 (Life Technologies). pCMVSport 3.0 contains an ampicillin resistance gene and may be transformed into E. coli strain DH10B, available from Life Technologies. (See, for instance, Gruber, C. E., et al., Focus 15:59-(1993).)

Two approaches can be used to isolate FGFR5 from the deposited sample. First, a specific polynucleotide of SEQ ID NO: 1 with 30-40 nucleotides is synthesized using an Applied Biosystems DNA synthesizer according to the sequence reported. The oligonucleotide is labeled, for instance, with ³²P-g-ATP using T4 polynucleotide kinase and purified according to routine methods. (E.g., Maniatis et al., Molecular Cloning: A Laboratory Manual, Cold Spring Harbor Press, Cold Spring, N.Y. (1982).) The plasmid mixture is transformed into a suitable host (such as XL-1 Blue (Stratagene)) using techniques known to those of skill in the art, such as those provided by the vector supplier or in related publications or patents. The transformants are plated on 1.5% agar plates (containing the appropriate selection agent, e.g., ampicillin) to a density of about 150 transformants (colonies) per plate. These plates are screened using Nylon membranes according to routine methods for bacterial colony screening (e.g., Sambrook et al., Molecular Cloning: A Laboratory Manual, 2nd Edit., (1989), Cold Spring Harbor Laboratory Press, pages 1.93 to 1.104), or other techniques known to those of skill in the art.

Alternatively, two primers of 17-20 nucleotides derived from both ends of the SEQ ID NO:1 (i.e., within the region of SEQ ID NO:1 bounded by the 5′ NT and the 3′ NT of the clone) are synthesized and used to amplify the FGFR5 cDNA using the deposited cDNA plasmid as a template. The polymerase chain reaction is carried out under routine conditions, for instance, in 25 ul of reaction mixture with 0.5 ug of the above cDNA template. A convenient reaction mixture is 1.5-5 mM MgCl₂, 0.01% (w/v) gelatin, 20 uM each of dATP, dCTP, dGTP, dTTP, 25 pmol of each primer and 0.25 Unit of Taq polymerase. Thirty five cycles of PCR (denaturation at 94 degree C. for 1 min; annealing at 55 degree C. for 1 min; elongation at 72 degree C. for 1 min) are performed with a Perkin-Elmer Cetus automated thermal cycler. The amplified product is analyzed by agarose gel electrophoresis and the DNA band with expected molecular weight is excised and purified. The PCR product is verified to be the selected sequence by subcloning and sequencing the DNA product.

Several methods are available for the identification of the 5′ or 3′ non-coding portions of the FGFR5 gene which may not be present in the deposited clone. These methods include but are not limited to, filter probing, clone enrichment using specific probes, and protocols similar or identical to 5′ and 3′ “RACE” protocols which are well known in the art. For instance, a method similar to 5′ RACE is available for generating the missing 5′ end of a desired full-length transcript. (Fromont-Racine et al., Nucleic Acids Res. 21(7):1683-1684 (1993).)

Briefly, a specific RNA oligonucleotide is ligated to the 5′ ends of a population of RNA presumably containing full-length gene RNA transcripts. A primer set containing a primer specific to the ligated RNA oligonucleotide and a primer specific to a known sequence of the FGFR5 gene of interest is used to PCR amplify the 5′ portion of the FGFR5 full-length gene. This amplified product may then be sequenced and used to generate the full length gene.

This above method starts with total RNA isolated from the desired source, although poly-A+ RNA can be used. The RNA preparation can then be treated with phosphatase if necessary to eliminate 5′ phosphate groups on degraded or damaged RNA which may interfere with the later RNA ligase step. The phosphatase should then be inactivated and the RNA treated with tobacco acid pyrophosphatase in order to remove the cap structure present at the 5′ ends of messenger RNAs. This reaction leaves a 5′ phosphate group at the 5′ end of the cap cleaved RNA which can then be ligated to an RNA oligonucleotide using T4 RNA ligase.

This modified RNA preparation is used as a template for first strand cDNA synthesis using a gene specific oligonucleotide. The first strand synthesis reaction is used as a template for PCR amplification of the desired 5′ end using a primer specific to the ligated RNA oligonucleotide and a primer specific to the known sequence of the gene of interest. The resultant product is then sequenced and analyzed to confirm that the 5′ end sequence belongs to the FGFR5 gene.

Alternatively, a genomic clone comprising the human FGFR5 coding exons can be isolated by screening a human genomic library as discussed infra. Once positive clones have been identified, the DNA inserts contained in the genomic clone can be isolated, and the DNA sequenced. Once the DNA sequence has been determined, the utilization of a number of computer-based DNA sequence analysis programs, such as, for example, BLAST and GRAIL, will allow the identification of the coding exons and the non-coding introns associated with the FGFR5 gene, and hence the identification of any 5′ portion of the FGFR5 full-length gene which may not have been previously present in the deposited clone.

### Example 2

### Isolation of FGFR5 Genomic Clones

A human genomic P1 library (Genomic Systems, Inc.) is screened by PCR using primers selected for the cDNA sequence corresponding to SEQ ID NO:1., according to the method described in Example 1. (See also, Sambrook.)

### Example 3

### Tissue Distribution of FGFR5 Polypeptides

Tissue distribution of mRNA expression of FGFR5 is determined using protocols for Northern blot analysis, described by, among others, Sambrook et al. For example, a FGFR5 probe produced by the method described in Example 1 is labeled with P³² using the rediprime™ DNA labeling system (Amersham Life Science), according to manufacturer's instructions. After labeling, the probe is purified using CHROMA SPIN-100™ column (Clontech Laboratories, Inc.), according to manufacturer's protocol number PT1200-1. The purified labeled probe is then used to examine various human tissues for mRNA expression.

Multiple Tissue Northern (MTN) blots containing various human tissues (H) or human immune system tissues (IM) (Clontech) are examined with the labeled probe using ExpressHyb™ hybridization solution (Clontech) according to manufacturer's protocol number PT1190-1. Following hybridization and washing, the blots are mounted and exposed to film at −70 degree C. overnight, and the films developed according to standard procedures.

### Example 4

### Chromosomal Mapping of FGFR5

An oligonucleotide primer set is designed according to the sequence at the 5′ end of SEQ ID NO:1. This primer preferably spans about 100 nucleotides. This primer set is then used in a polymerase chain reaction under the following set of conditions: 30 seconds, 95 degree C.; 1 minute, 56 degree C.; 1 minute, 70 degree C. This cycle is repeated 32 times followed by one 5 minute cycle at 70 degree C. Human, mouse, and hamster DNA is used as template in addition to a somatic cell hybrid panel containing individual chromosomes or chromosome fragments (Bios, Inc). The reactions is analyzed on either 8% polyacrylamide gels or 3.5% agarose gels. Chromosome mapping is determined by the presence of an approximately 100 bp PCR fragment in the particular somatic cell hybrid.

### Example 5

### Bacterial Expression of Mature Extracellular Domain of FGFR5

FGFR5 polynucleotide encoding a FGFR5 polypeptide of the invention is amplified using PCR oligonucleotide primers corresponding to the 5′ and 3′ ends of the DNA sequence, as outlined in Example 1, to synthesize insertion fragments. The primers used to amplify the cDNA insert should preferably contain restriction sites, such as BamHI and XbaI, at the 5′ end of the primers in order to clone the amplified product into the expression vector. For example, BamHI and XbaI correspond to the restriction enzyme sites on the bacterial expression vector pQE-9. (Qiagen, Inc., Chatsworth, Calif.). This plasmid vector encodes antibiotic resistance (Ampr), a bacterial origin of replication (ori), an IPTG-regulatable promoter/operator (P/O), a ribosome binding site (RBS), a 6-histidine tag (6-His), and restriction enzyme cloning sites.

Specifically, to clone the FGFR5 polypeptide in a bacterial vector, the 5′ primer has the sequence 5′ GCAGCACATATGGCCCGAGGCCCCCCAAAGATGGCGGAC 3′ (SEQ ID NO: 4) containing the underlined NdeI restriction site followed by a number of nucleotides of the amino terminal coding sequence of the mature extracellular domain of the FGFR5 sequence in SEQ ID NO:1. One of ordinary skill in the art would appreciate, of course, that the point in the protein coding sequence where the 5′ primer begins may be varied to amplify a DNA segment encoding any desired portion of the complete FGFR5 protein shorter or longer than the full-length form of the protein. The 3′ primer has the sequence 5′GCAGCAGGTACCTTAAGTGGCCGAGGACGAGGAGGCCACAGGTGGC 3′ (SEQ ID NO: 5) containing the underlined Asp718 restriction site followed by a number of nucleotides complementary to the 3′ end of the coding sequence of the mature extracellular domain of the FGFR5 DNA sequence of SEQ ID NO:1.

The pQE-9 vector is digested with NdeI and Asp718 and the amplified fragment is ligated into the pQE-9 vector maintaining the reading frame initiated at the bacterial RBS. The ligation mixture is then used to transform the E. coli strain M15/rep4 (Qiagen, Inc.) which contains multiple copies of the plasmid pREP4, which expresses the lacd repressor and also confers kanamycin resistance (Kan^{r}). Transformants are identified by their ability to grow on LB plates and ampicillin/kanamycin resistant colonies are selected. Plasmid DNA is isolated and confirmed by restriction analysis.

Clones containing the desired constructs are grown overnight (O/N) in liquid culture in LB media supplemented with both Amp (100 ug/ml) and Kan (25 ug/ml). The O/N culture is used to inoculate a large culture at a ratio of 1:100 to 1:250. The cells are grown to an optical density 600 (O.D.⁶⁰⁰) of between 0.4 and 0.6. IPTG (Isopropyl-B-D-thiogalacto pyranoside) is then added to a final concentration of 1 mM. IPTG induces by inactivating the lac repressor, clearing the P/O leading to increased gene expression.

Cells are grown for an extra 3 to 4 hours. Cells are then harvested by centrifugation (20 mins at 6000×g).

In addition to the above expression vector, the present invention further includes an expression vector comprising phage operator and promoter elements operatively linked to a FGFR5 polynucleotide, called pHE4a. (ATCC Accession Number 209645, deposited Feb. 25, 1998.) This vector contains: 1) a neomycinphosphotransferase gene as a selection marker, 2) an E. coli origin of replication, 3) a T5 phage promoter sequence, 4) two lac operator seqeunces, 5) a Shine-Delgarno sequence, and 6) the lactose operon repressor gene (lacIq). The origin of replication (oriC) is derived from pUC19 (LTI, Gaithersburg, Md.). The promoter sequence and operator sequences are made synthetically.

DNA can be inserted into the pHEa by restricting the vector with NdeI and KpnI, BamHI, XhoI, or Asp718, running the restricted product on a gel, and isolating the larger fragment (the stuffer fragment should be about 310 base pairs). The DNA insert is generated according to the PCR protocol described in Example 1, using PCR primers having restriction sites for NdeI (5′ primer) and XbaI, BamHI, XhoI, or Asp718 (3′ primer). The PCR insert is gel purified and restricted with compatible enzymes. The insert and vector are ligated according to standard protocols.

The engineered vector could easily be substituted in the above protocol to express protein in a bacterial system.

### Example 6

### Purification of FGFR5 Polypeptide from an Inclusion Body

The following alternative method can be used to purify FGFR5 polypeptide expressed in E. coli when it is present in the form of inclusion bodies. Unless otherwise specified, all of the following steps are conducted at 4-10 degree C.

Upon completion of the production phase of the E. coli fermentation, the cell culture is cooled to 4-10 degree C. and the cells harvested by continuous centrifugation at 15,000 rpm (Heraeus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells are then lysed by passing the solution through a microfluidizer (Microfuidics, Corp. or APV Gaulin, Inc.) twice at 4000-6000 psi. The homogenate is then mixed with NaCl solution to a final concentration of 0.5 M NaCl, followed by centrifugation at 7000×g for 15 min. The resultant pellet is washed again using 0.5M NaCl, 100 mM Tris, 50 mM EDTA, pH 7.4.

The resulting washed inclusion bodies are solubilized with 1.5 M guanidine hydrochloride (GuHCl) for 2-4 hours. After 7000×g centrifugation for 15 min., the pellet is discarded and the polypeptide containing supernatant is incubated at 4 degree C. overnight to allow further GuHCl extraction.

Following high speed centrifugation (30,000×g) to remove insoluble particles, the GCuHCl solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4 degree C. without mixing for 12 hours prior to further purification steps.

To clarify the refolded polypeptide solution, a previously prepared tangential filtration unit equipped with 0.16 um membrane filter with appropriate surface area (e.g., Filtron), equilibrated with 40 mM sodium acetate, pH 6.0 is employed. The filtered sample is loaded onto a cation exchange resin (e.g., Poros HS-50, Perseptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 nm of the effluent is continuously monitored. Fractions are collected and further analyzed by SDS-PAGE.

Fractions containing the FGFR5 polypeptide are then pooled and mixed with 4 volumes of water. The diluted sample is then loaded onto a previously prepared set of tandem columns of strong anion (Poros HQ-50, Perseptive Biosystems) and weak anion (Poros CM-20, Perseptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A₂₈₀ monitoring of the effluent. Fractions containing the polypeptide (determined, for instance, by 16% SDS-PAGE) are then pooled.

The resultant FGFR5 polypeptide should exhibit greater than 95% purity after the above refolding and purification steps. No major contaminant bands should be observed from Commassie blue stained 16% SDS-PAGE gel when 5 ug of purified protein is loaded. The purified FGFR5 protein can also be tested for endotoxin/LPS contamination, and typically the LPS content is less than 0.1 ng/ml according to LAL assays.

### Example 7

### Cloning and Expression of FGFR5 in a Baculovirus Expression System

In this example, the plasmid shuttle vector pA2 is used to insert FGFR5 polynucleotide into a baculovirus to express FGFR5. This expression vector contains the strong polyhedrin promoter of the Autographa californica nuclear polyhedrosis virus (AcMNPV) followed by convenient restriction sites such as BamHI, Xba I and Asp718. The polyadenylation site of the simian virus 40 (“SV40”) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that expresses the cloned FGFR5 polynucleotide.

Many other baculovirus vectors can be used in place of the vector above, such as pAc373, pVL941, and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow et al., Virology 170:31-39(1389).

Specifically, the FGFR5 cDNA sequence contained in the deposited clone, including the AUG initiation codon and any naturally associated leader sequence, is amplified using the PCR protocol described in Example 1. If the naturally occurring signal sequence is used to produce the secreted protein, the pA2 vector does not need a second signal peptide. Alternatively, the vector can be modified (pA2 GP) to include a baculovirus leader sequence, using the standard methods described in Summers et al., “A Manual of Methods for Baculovirus Vectors and Insect Cell Culture Procedures,” Texas Agricultural Experimental Station Bulletin No. 1555 (1987).

More specifically, the cDNA sequence encoding the full length FGFR5 protein shown in SEQ ID NO:1, is amplified using PCR oligonucleotide primers corresponding to the 5′ and 3′ sequences of the gene. The 5′ primer has the sequence 5′ GCAGCAGGATCCGCCATCATGACGCCGAGCCCCCTGTTGCTGCTCCTG 3′ (SEQ ID NO: 6) containing the BamHI restriction enzyme site, an efficient signal for initiation of translation in eukaryotic cells (Kozak, M., J. Mol. Biol. 196:947-950 (1987)), followed by a number of nucleotides of the sequence of the FGFR5 protein shown in FIGS. 1A-C. The 3′ primer has the sequence 5′ GCAGCAGGTACCTTAGCACTGATAGTGGATGTGCTGGTGGACCTTG 3′ (SEQ ID NO: 7) containing the KpnI restriction site followed by a number of nucleotides complementary to the 3′ sequence in FIGS. 1A-C. Alternatively, the following 3′ primer may be used to amplify the full-length extracellular domain of the FGFR5 protein shown in SEQ ID NO:1. The 3′ primer has the sequence 5′ GCAGCAGGTACCTTAAGTGGCCGAGGACGAGGAGGCCACAGGTGGC 3′ (SEQ ID NO: 14)

The amplified fragment is isolated from a 1% agarose gel using a commercially available kit (“Geneclean,” BIO 101 Inc., La Jolla, Calif.). The fragment then is digested with appropriate restriction enzymes and again purified on a 1% agarose gel.

The plasmid is digested with the corresponding restriction enzymes and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.).

The fragment and the dephosphorylated plasmid are ligated together with T4 DNA, ligase. E. coli HB101 or other suitable E. coli hosts such as XL-1 Blue (Stratagene Cloning Systems, La Jolla, Calif.) cells are transformed with the ligation mixture and spread on culture plates. Bacteria containing the plasmid are identified by digesting DNA from individual colonies and analyzing the digestion product by gel electrophoresis. The sequence of the cloned fragment is confirmed by DNA sequencing.

Five ug of a plasmid containing the polynucleotide is co-transfected with 1.0 ug of a commercially available linearized baculovirus DNA (“BaculoGold™ baculovirus DNA”, Pharmingen, San Diego, Calif.), using the lipofection method described by Felgner et al., Proc. Natl. Acad. Sci. USA 84:7413-7417 (1987). One ug of BaculoGold™ virus DNA and 5 ug of the plasmid are mixed in a sterile well of a microtiter plate containing 50 ul of serum-free Grace's medium (Life Technologies Inc., Gaithersburg, Md.). Afterwards, 10 ul Lipofectin plus 90 ul Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is then incubated for 5 hours at 27 degrees C. The transfection solution is then removed from the plate and 1 ml of Grace's insect medium supplemented with 10% fetal calf serum is added. Cultivation is then continued at 27 degrees C. for four days.

After four days the supernatant is collected and a plaque assay is performed, as described by Summers and Smith, supra. An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10.) After appropriate incubation, blue stained plaques are picked with the tip of a micropipettor (e.g., Eppendorf). The agar containing the recombinant viruses is then resuspended in a microcentrifuge tube containing 200 ul of Grace's medium and the suspension containing the recombinant baculovirus is used to infect Sf9 cells seeded in 35 mm dishes. Four days later the supernatants of these culture dishes are harvested and then they are stored at 4 degree C.

To verify the expression of the polypeptide, Sf9 cells are grown in Grace's medium supplemented with 10% heat-inactivated FBS. The cells are infected with the recombinant baculovirus containing the polynucleotide at a multiplicity of infection (“MOI”) of about 2. If radiolabeled proteins are desired, 6 hours later the medium is removed and is replaced with SF900 II medium minus methionine and cysteine (available from Life Technologies Inc., Rockville, Md.). After 42 hours, 5 uCi of ³⁵S-methionine and 5 uCi ³⁵S-cysteine (available from Amersham) are added. The cells are further incubated for 16 hours and then are harvested by centrifugation. The proteins are analyzed by SDS-PAGE followed by autoradiography (if radiolabeled).

Microsequencing of the amino acid sequence of the amino terminus of purified protein may be used to determine the amino terminal sequence of the produced FGFR5 polypeptide.

### Example 8

### Expression of FGFR5 in Mammalian Cells

FGFR5 polypeptide can be expressed in a mammalian cell. A typical mammalian expression vector contains a promoter element, which mediates the initiation of transcription of mRNA, a protein coding sequence, and signals required for the termination of transcription and polyadenylation of the transcript. Additional elements include enhancers, Kozak sequences and intervening sequences flanked by donor and acceptor sites for RNA splicing. Highly efficient transcription is achieved with the early and late promoters from SV40, the long terminal repeats (LTRs) from Retroviruses, e.g., RSV, HTLVI, HIVI and the early promoter of the cytomegalovirus (CMV). However, cellular elements can also be used (e.g., the human actin promoter).

Suitable expression vectors for use in practicing the present invention include, for example, vectors such as pSVL and pMSG (Pharmacia, Uppsala, Sweden), pRSVcat (ATCC 37152), pSV2DHFR (ATCC 37146), pBC12MI (ATCC 67109), pCMVSport 2.0, and pCMVSport 3.0. Mammalian host cells that could be used include, human Hela, 293, H9 and Jurkat cells, mouse NIH3T3 and C127 cells, Cos 1, Cos 7 and CV1, quail QC1-3 cells, mouse L cells and Chinese hamster ovary (CHO) cells.

Alternatively, FGFR5 polypeptide can be expressed in stable cell lines containing the FGFR5 polynucleotide integrated into a chromosome. The co-transfection with a selectable marker such as DHFR, gpt, neomycin, hygromycin allows the identification and isolation of the transfected cells.

The transfected FGFR5 gene can also be amplified to express large amounts of the encoded protein. The DHFR (dihydrofolate reductase) marker is useful in developing cell lines that carry several hundred or even several thousand copies of the gene of interest. (See, e.g., Alt, F. W., et al., J. Biol. Chem. 253:1357-1370 (1978); Hamlin, J. L. and Ma, C., Biochem. et Biophys. Acta, 1097:107-143 (1990); Page, M. J. and Sydenham, M. A., Biotechnology 9:64-68 (1991).) Another useful selection marker is the enzyme glutamine synthase (GS) (Murphy et al., Biochem J. 227:277-279 (1991); Bebbington et al., Bio/Technology 10:169-175 (1992). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

Derivatives of the plasmid pSV2-DHFR (ATCC Accession No. 37146), the expression vectors pC4 (ATCC Accession No. 209646) and pC6 (ATCC Accession No.209647) contain the strong promoter (LTR) of the Rous Sarcoma Virus (Cullen et al., Molecular and Cellular Biology, 438-447 (March, 1985)) plus a fragment of the CMV-enhancer (Boshart et al., Cell 41:521-530 (1985).) Multiple cloning sites, e.g., with the restriction enzyme cleavage sites BamHI, XbaI and Asp718, facilitate the cloning of FGFR5. The vectors also contain the 3′ intron, the polyadenylation and termination signal of the rat preproinsulin gene, and the mouse DHFR gene under control of the SV40 early promoter.

Specifically, the plasmid pC4 is digested with BamHI and KpnI and then dephosphorylated using calf intestinal phosphates by procedures known in the art. The vector is then isolated from a 1% agarose gel.

The cDNA sequence encoding the FGFR5 protein in the deposited clone is amplified using PCR oligonucleotide primers corresponding to the 5′ and 3′ sequences of the gene. The 5′ primer has the sequence 5′ GCAGCAGGATCC GCCATCATGACGAAGAGCCCCCTGTTGCTGCTCCTG 3′ (SEQ ID NO: 8) containing the BamHI restriction enzyme site, an efficient signal for initiation of translation in eukaryotic cells (Kozak, M., J. Mol. Biol. 196:947-950 (1987)), followed by a number of nucleotides of the sequence of the FGFR5 protein shown in FIGS. 1A-C. In this embodiment, the 3′ primer has the sequence 5′ GCAGCAGGTACCTTAGCACTGATAGTGTATGTGCTGGTGGACCTTG 3′ (SEQ ID NO: 9) containing the KpnI restriction site followed by a number of nucleotides complementary to the 3′ sequence in FIGS. 1A-C. Alternatively, the following 3′ primer may be used to amplify the full-length extracellular domain of the FGFR5 protein shown in SEQ ID NO:1. The 3′ primer has the sequence 5′ GCAGCAGGTACCTTAAGTGGCCGAGGACGAGGAGGCCACAGGTGGC 3′ (SEQ ID NO: 15)

In one specific embodiment, the 5′ primer has the sequence 5′ GCAGCAGGATCCCGCCATCAACGCCACCTACAAGGTGGATGT 3′ (SEQ ID NO: 10) containing the BamHI restriction enzyme site, an efficient signal for initiation of translation in eukaryotic cells (Kozak, M., J. Mol. Biol. 196:947-950 (1987)), followed by a number of nucleotides of the sequence of the FGFR5 protein shown in FIGS. 1A-C. In this embodiment, the 3′ primer has the sequence 5′ GCAGCAGGTACCTTAAGTGGCCGAGGACGAGGAGGCCACAGGTGGC 3′ (SEQ ID NO: 11) containing the KpnI restriction site followed by a number of nucleotides complementary to the 3′ sequence in FIGS. 1A-C.

If a naturally occurring signal sequence is used to produce a secreted protein, the vector does not need a second signal peptide. Alternatively, if a naturally occurring signal sequence is not used, the vector can be modified to include a heterologous signal sequence in an effort to secrete the protein from the cell. (See, e.g., WO 96/34891.)

The amplified fragment is then digested with the BamHI and KpnI and purified on a 1% agarose gel using a commercially available kit (“Geneclean,” BIO 101 Inc., La Jolla, Calif.). The isolated fragment and the dephosphorylated vector are then ligated with T4 DNA ligase. E. coli HB101 or XL-1 Blue cells are then transformed and bacteria are identified that contain the fragment inserted into plasmid pC4 using, for instance, restriction enzyme analysis.

Chinese hamster ovary cells lacking an active DHFR gene is used for transfection. Five μg of the expression plasmid pC4 is cotransfected with 0.5 ug of the plasmid pSVneo using lipofectin (Felgner et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of metothrexate plus I mg/mil G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 uM, 2 uM, 5 uM, 10 mM, 20 mM). The same procedure is repeated until clones are obtained which grow at a concentration of 100-200 uM. Expression of FGFR5 is analyzed, for instance, by SDS-PAGE and Western blot or by reversed phase HPLC analysis.

### Example 9

### Construction of N-Terminal and/or C-Terminal Deletion Mutants

The following general approach may be used to clone a N-terminal or C-terminal deletion FGFR5 deletion mutant. Generally, two oligonucleotide primers of about 15-25 nucleotides are derived from the desired 5′ and 3′ positions of a polynucleotide of SEQ ID NO:1. The 5′ and 3′ positions of the primers are determined based on the desired FGFR5 polynucleotide fragment. An initiation and stop codon are added to the 5′ and 3′ primers respectively, if necessary, to express the FGFR5 polypeptide fragment encoded by the polynucleotide fragment. Preferred FGFR5 polynucleotide fragments are those encoding the N-terminal and C-terminal deletion mutants disclosed above in the “Polynucleotide and Polypeptide Fragments” section of the Specification.

Additional nucleotides containing restriction sites to facilitate cloning of the FGFR5 polynucleotide fragment in a desired vector may also be added to the 5′ and 3′ primer sequences. The FGFR5 polynucleotide fragment is amplified from genomic DNA or from the deposited cDNA clone using the appropriate PCR oligonucleotide primers and conditions discussed herein or known in the art. The FGFR5 polypeptide fragments encoded by the FGFR5 polynucleotide fragments of the present invention may be expressed and purified in the same general manner as the full length polypeptides, although routine modifications may be necessary due to the differences in chemical and physical properties between a particular fragment and full length polypeptide.

As a means of exemplifying but not limiting the present invention, the polynucleotide encoding the FGFR5 polypeptide fragment V1 to P-185 is amplified and cloned as follows: A 5′ primer is generated comprising a restriction enzyme site followed by an initiation codon in frame with the polynucleotide sequence encoding the N-terminal portion of the polypeptide fragment beginning with V-1. A complementary 3′ primer is generated comprising a restriction enzyme site followed by a stop codon in frame with the polynucleotide sequence encoding C-terminal portion of the FGFR5 polypeptide fragment ending with P-185.

The amplified polynucleotide fragment and the expression vector are digested with restriction enzymes which recognize the sites in the primers. The digested polynucleotides are then ligated together. The FGFR5 polynucleotide fragment is inserted into the restricted expression vector, preferably in a manner which places the FGFR5 polypeptide fragment coding region downstream from the promoter. The ligation mixture is transformed into competent E. coli cells using standard procedures and as described in the Examples herein. Plasmid DNA is isolated from resistant colonies and the identity of the cloned DNA confirmed by restriction analysis, PCR and DNA sequencing.

### Example 10

### Protein Fusions of FGFR5

FGFR5 polypeptides are preferably fused to other proteins. These fusion proteins can be used for a variety of applications. For example, fusion of FGFR5 polypeptides to His-tag, HA-tag, protein A, IgG domains, and maltose binding protein facilitates purification. (See Example 5; see also EP A 394,827; Traunecker, et al., Nature 331:84-86 (1988).) Similarly, fusion to IgG-1, IgG-3, and albumin increases the halflife time in vivo. Nuclear localization signals fused to FGFR5 polypeptides can target the protein to a specific subcellular localization, while covalent heterodimer or homodimers can increase or decrease the activity of a fusion protein. Fusion proteins can also create chimeric molecules having more than one function. Finally, fusion proteins can increase solubility and/or stability of the fused protein compared to the non-fused protein. All of the types of fusion proteins described above can be made by modifying the following protocol, which outlines the fusion of a polypeptide to an IgG molecule.

Briefly, the human Fc portion of the IgG molecule can be PCR amplified, using primers that span the 5′ and 3′ ends of the sequence described below. These primers also should have convenient restriction enzyme sites that will facilitate cloning into an expression vector, preferably a mammalian expression vector.

For example, if pC4 (Accession No. 209646) is used, the human Fc portion can be ligated into the BamHI cloning site. Note that the 3′ BamHI site should be destroyed. Next, the vector containing the human Fc portion is re-restricted with BamHI, linearizing the vector, and FGFR5 polynucleotide, isolated by the PCR protocol described in Example 1, is ligated into this BamHI site. Note that the polynucleotide is cloned without a stop codon, otherwise a fusion protein will not be produced.

If the naturally occurring signal sequence is used to produce the secreted protein, pC4 does not need a second signal peptide. Alternatively, if the naturally occurring signal sequence is not used, the vector can be modified to include a heterologous signal sequence. (See, e.g., WO 96/34891.)

Human IgG Fc region:

GGGATCCGGAGCCCAAATCTTCTGACAAAACTCACACATGCCCACCGTGC CCAGCACCTGAATTCGAGGGTGCACCGTCAGTCTTCCTCTTCCCCCCAAAA CCCAAGGACACCCTCATGATCTCCCGGACTCCTGAGGTCACATGCGTGGT GGTGGACGTAAGCCACGAAGACCCTGAGGTCAAGTTCAACTGGTACGTGG ACGGCGTGGAGGTGCATAATGCCAAGACAAAGCCGCGGGAGGAGCAGTA CAACAGCACGTACCGTGTGGTCAGCGTCCTCACCGTCCTGCACCAGGACT GGCTGAATGGCAAGGAGTACAAGTGCAAGGTCTCCAACAAAGCCCTCCCA ACCCCCATCGAGAAAACCATCTCCAAAGCCAAAGGGCAGCCCCGAGAAC CACAGGTGTACACCCTGCCCCCATCCCGGGATGAGCTGACCAAGAACCAG GTCAGCCTGACCTGCCTGGTCAAAGGCTTCTATCCAAGCGACATCGCCGT GGAGTGGGAGAGCAATGGGCAGCCGGAGAACAACTACAAGACCACGCCT CCCGTGCTGGACTCCGACGGCTCCTTCTTCCTCTACAGCAAGCTCACCGTG GACAAGAGCAGGTGGCAGCAGGGGAACGTCTTCTCATGCTCCGTGATGCA TGAGGCTCTGCACAACCACTACACGCAGAAGAGCCTCTCCCTGTCTCCGG GTAAATGAGTGCGACGGCCGCGACTCTAGAGGAT (SEQ ID NO:12)

### Example 11

### Production of an Antibody

The antibodies of the present invention can be prepared by a variety of methods. (See, Current Protocols, Chapter 2.) For example, cells expressing FGFR5 will be administered to an animal to induce the production of sera containing polyclonal antibodies. In a preferred method, a preparation of FGFR5 protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or protein binding fragments thereof). Such monoclonal antibodies can be prepared using hybridoma technology. (Köhler et al., Nature 256:495 (1975); Köhler et al., Eur. J. Immunol. 6:511 (1976); Köhler et al., Eur. J. Immunol. 6:292 (1976); Hammerling et al., in: Monoclonal Antibodies and T-Cell Hybridomas, Elsevier, N.Y., pp. 563-681 (1981).) In general, such procedures involve immunizing an animal (preferably a mouse) with FGFR5 polypeptide or, more preferably, with a secreted FGFR5 polypeptide-expressing cell. Such cells may be cultured in any suitable tissue culture medium; however, it is preferable to culture cells in Earle's modified Eagle's medium supplemented with 10% fetal bovine serum (inactivated at about 56 degree C.), and supplemented with about 10 g/l of nonessential amino acids, about 1,000 U/ml of penicillin, and about 100 ug/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP2O), available from the ATCC. After fusion, the resulting hybridoma cells are selectively maintained in HAT medium, and then cloned by limiting dilution as described by Wands et al. (Gastroenterology 80:225-232 (1981).) The hybridoma cells obtained through such a selection are then assayed to identify clones which secrete antibodies capable of binding the FGFR5 polypeptide.

Alternatively, additional antibodies capable of binding to FGFR5 polypeptide can be produced in a two-step procedure using anti-idiotypic antibodies. Such a method makes use of the fact that antibodies are themselves antigens, and therefore, it is possible to obtain an antibody which binds to a second antibody. In accordance with this method, protein specific antibodies are used to immunize an animal, preferably a mouse. The splenocytes of such an animal are then used to produce hybridoma cells, and the hybridoma cells are screened to identify clones which produce an antibody whose ability to bind to the FGFR5 specific antibody can be blocked by FGFR5 Such antibodies comprise anti-idiotypic antibodies to the FGFR5 specific antibody and can be used to immunize an animal to induce formation of further FGFR5 specific antibodies.

It will be appreciated that Fab and F(ab′)2 and other fragments of the antibodies of the present invention may be used according to the methods disclosed herein. Such fragments are typically produced by proteolytic cleavage, using enzymes such as papain (to produce Fab fragments) or pepsin (to produce F(ab′)2 fragments). Alternatively, secreted FGFR5 protein-binding fragments can be produced through the application of recombinant DNA technology or through synthetic chemistry.

For in vivo use of antibodies in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art. (See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).)

### Example 12

### Isolation of Antibody Fragments Directed Against Polypeptides of the Present Invention from a Library of scFvs

Naturally occuring V-genes isolated from human PBLs are constructed into a large library of antibody fragments which contain reactivities against polypeptide of the present invention to which the donor may or may not have been exposed (see e.g., U.S. Pat. No. 5,885,793 incorporated herein in its entirety by reference).

A. Rescue of the library

A library of scFvs is constructed from the RNA of human PBLs as described in WO92/01047. To rescue phage displaying antibody fragments, approximately 10⁹ E. coli harbouring the phagemid are used to inoculate 50 ml of 2×TY containing 1% glucose and 100 mg/ml of ampicillin (2×TY-AMP-GLU) and grown to an O.D. of 0.8 with shaking. Five ml of this culture is used to innoculate 50 ml of 2×TY-AMP-GLU, 2.times.10⁸ TU of delta gene 3 helper (M13 D gene III, see WO92/01047) are added and the culture incubated at 37° C. for 45 minutes without shaking and then at 37° C. for 45 minutes with shaking. The culture is centrifuged at 4000 r.p.m. for 10 min. and the pellet resuspended in 2 liters of of 2×TY containing 100 mg/ml ampicillin and 50 mg/mil kanamycin and grown overnight. Phage are prepared as described in WO92/01047.

M13 D gene III is prepared as follows: M13 D gene III helper phage does not encode gene III protein, hence the phage(mid) displaying antibody fragments have a greater avidity of binding to antigen. Infectious M13 D gene III particles are made by growing the helper phage in cells harbouring a pUC19 derivative supplying the wild type gIII protein during phage morphogenesis. The culture is incubated for 1 hour at 37° C. without shaking and then for a further hour at 37° C. with shaking. Cells were spun down (IEC-Centra 8, 4000 revs/min for 10 min), resuspended in 300 ml 2×TY broth containing 100 mg ampicillin/ml and 25 mg kanamycin/ml (2×TY-AMP-KAN) and grown overnight, shaking at 37° C. Phage particles are purified and concentrated from the culture medium by two PEG-precipitations (Sambrook et al., 1990), resuspended in 2 ml PBS and passed through a 0.45 mm filter (Minisart NML; Sartorius) to give a final concentration of approximately 10¹³ transducing units/ml (ampicillin-resistant clones).

B. Panning of the library

Immunotubes (Nunc) are coated overnight in PBS with 4 ml of either 100 mg/ml or 10 mg/ml of a polypeptide of the present invention. Tubes are blocked with 2% Marvel-PBS for 2 hours at 37° C. and then washed 3 times in PBS. Approximately 10¹³ TU of phage is applied to the tube and incubated for 30 minutes at room temperature tumbling on an over and under turntable and then left to stand for another 1.5 hours. Tubes are washed 10 times with PBS 0.1% Tween-20 and 10 times with PBS. Phage are eluted by adding 1 ml of 100 mM triethylamine and rotating 15 minutes on an under and over turntable after which the solution is immediately neutralized with 0.5 ml of 1,0M Tris-HCl, pH 7.4. Phage are then used to infect 10 ml of mid-log E. coli TG1 by in+cubating eluted phage with bacteria for 30 minutes at 37° C. The E. coli are then plated on TYE plates containing 1% glucose and 100 mg/ml ampicillin. The resulting bacterial library is then rescued with delta gene 3 helper phage as described above to prepare phage for a subsequent round of selection. This process is then repeated for a total of 4 rounds of affinity purification with tube-washing increased to 20 times with PBS, 0.1% Tween-20 and 20 times with PBS for rounds 3 and 4.

C. Characterization of binders

Eluted phage from the 3rd and 4th rounds of selection are used to infect E. coli HB 2151 and soluble scFv is produced (Marks, et al., 1991) from single colonies for assay. ELISAs are performed with microtitre plates coated with either 10 pg/ml of the polypeptide of the present invention in 50 mM bicarbonate pH 9.6. Clones positive in ELISA are further characterized by PCR fingerprinting (see e.g., WO92/01047) and then by sequencing.

### Example 13

### Method of Determining Alterations in the FGFR5 Gene

RNA isolated from entire families or individual patients presenting with a phenotype of interest (such as a disease) is be isolated. cDNA is then generated from these RNA samples using protocols known in the art. (See, Sambrook.) The cDNA is then used as a template for PCR, employing primers surrounding regions of interest in SEQ ID NO:1. Suggested PCR conditions consist of 35 cycles at 95 degree C. for 30 seconds; 60-120 seconds at 52-58 degree C.; and 60-120 seconds at 70 degree C., using buffer solutions described in Sidransky, D., et al., Science 252:706 (1991).

PCR products are then sequenced using primers labeled at their 5′ end with T4 poly nucleotide kinase, employing SequiTherm Polymerase. (Epicentre Technologies). The intron-exon borders of selected exons of FGFR5 is also determined and genomic PCR products analyzed to confirm the results. PCR products harboring suspected mutations in FGFR5 is then cloned and sequenced to validate the results of the direct sequencing.

PCR products of FGFR5 are cloned into T-tailed vectors as described in Holton, T. A. and Graham, M. W., Nucleic Acids Research, 19:1156 (1991) and sequenced with T7 polymerase (United States Biochemical). Affected individuals are identified by mutations in FGFR5 not present in unaffected individuals.

Genomic rearrangements are also observed as a method of determining alterations in the FGFR5 gene. Genomic clones isolated according to Example 2 are nick-translated with digoxigenindeoxy-uridine 5′-triphosphate (Boehringer Manheim), and FISH performed as described in Johnson, Cg. et al., Methods Cell Biol. 35:73-99 (1991). Hybridization with the labeled probe is carried out using a vast excess of human cot-1 DNA for specific hybridization to the FGFR5 genomic locus.

Chromosomes are counterstained with 4,6-diamino-2-phenylidole and propidium iodide, producing a combination of C- and R-bands. Aligned images for precise mapping are obtained using a triple-band filter set (Chroma Technology, Brattleboro, Vt.) in combination with a cooled charge-coupled device camera (Photometrics, Tucson, Ariz.) and variable excitation wavelength filters. (Johnson, Cv. et al., Genet. Anal. Tech. Appl., 8:75 (1991).) Image collection, analysis and chromosomal fractional length measurements are performed using the ISee Graphical Program System. (Inovision Corporation, Durham, N.C.) Chromosome alterations of the genomic region of FGFR5 (hybridized by the probe) are identified as insertions, deletions, and translocations. These FGFR5 alterations are used as a diagnostic marker for an associated disease.

### Example 14

### Method of Detecting Abnormal Levels of FGFR5 in a Biological Sample

FGFR5 polypeptides can be detected in a biological sample, and if an increased or decreased level of FGFR5 is detected, this polypeptide is a marker for a particular phenotype. Methods of detection are numerous, and thus, it is understood that one skilled in the art can modify the following assay to fit their particular needs.

For example, antibody-sandwich ELISAs are used to detect FGFR5 in a sample, preferably a biological sample. Wells of a microtiter plate are coated with specific antibodies to FGFR5 at a final concentration of 0.2 to 10 ug/ml. The antibodies are either monoclonal or polyclonal and are produced by the method described in Example 11. The wells are blocked so that non-specific binding of FGFR5 to the well is reduced.

The coated wells are then incubated for >2 hours at RT with a sample containing FGFR5. Preferably, serial dilutions of the sample should be used to validate results. The plates are then washed three times with deionized or distilled water to remove unbounded FGFR5.

Next, 50 ul of specific antibody-alkaline phosphatase conjugate, at a concentration of 25-400 ng, is added and incubated for 2 hours at room temperature. The plates are again washed three times with deionized or distilled water to remove unbounded conjugate.

Add 75 ul of 4-methylumbelliferyl phosphate (MUP) or p-nitrophenyl phosphate (NPP) substrate solution to each well and incubate 1 hour at room temperature. Measure the reaction by a microtiter plate reader. Prepare a standard curve, using serial dilutions of a control sample, and plot FGFR5 polypeptide concentration on the X-axis (log scale) and fluorescence or absorbance of the Y-axis (linear scale). Interpolate the concentration of the FGFR5 in the sample using the standard curve.

### Example 15

### Formulating a Polypeptide

The FGFR5 composition will be formulated and dosed in a fashion consistent with good medical practice, taking into account the clinical condition of the individual patient (especially the side effects of treatment with the FGFR5 polypeptide alone), the site of delivery, the method of administration, the scheduling of administration, and other factors known to practitioners. The “effective amount” for purposes herein is thus determined by such considerations.

As a general proposition, the total pharmaceutically effective amount of FGFR5 administered parenterally per dose will be in the range of about 1 ug/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, FGFR5 is typically administered at a dose rate of about 1 ug/kg/hour to about 50 ug/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Pharmaceutical compositions containing FGFR5 are administered orally, rectally, parenterally, intracistemally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

FGFR5 is also suitably administered by sustained-release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (Sidman, U. et al., Biopolymers 22:547-556 (1983)), poly (2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al.) or poly-D-(−)-3-hydroxybutyric acid (EP 133,988). Sustained-release compositions also include liposomally entrapped FGFR5 polypeptides. Liposomes containing the FGFR5 are prepared by methods known per se: DE 3,218,121; Epstein et al., Proc. Natl. Acad. Sci. USA 82:3688-3692 (1985); Bwang et al., Proc. Natl. Acad. Sci. USA 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal secreted polypeptide therapy.

For parenteral administration, in one embodiment, FGFR5 is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting FGFR5 uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

FGFR5 is typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of polypeptide salts.

FGFR5 used for therapeutic administration can be sterile. Sterility is readily accomplished by filtration through sterile filtration membranes (e.g., 0.2 micron membranes). Therapeutic polypeptide compositions generally are placed into a container having a sterile access port, for example, an intravenous solution bag or vial having a stopper pierceable by a hypodermic injection needle.

FGFR5 polypeptides ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous FGFR5 polypeptide solution, and the resulting mixture is lyophilized. The infusion solution is prepared by reconstituting the lyophilized FGFR5 polypeptide using bacteriostatic Water-for-Injection.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, FGFR5 may be employed in conjunction with other therapeutic compounds.

### Example 16

### Method of Treatment Using Gene Therapy—In Vivo

Another aspect of the present invention is using in vivo gene therapy methods to treat disorders, diseases and conditions. The gene therapy method relates to the introduction of naked nucleic acid (DNA, RNA, and antisense DNA or RNA) FGFR5 sequences into an animal to increase or decrease the expression of the FGFR5 polypeptide. The FGFR5 polynucleotide may be operatively linked to a promoter or any other genetic elements necessary for the expression of the FGFR5 polypeptide by the target tissue. Such gene therapy and delivery techniques and methods are known in the art, see, for example, WO90/11092, WO98/11779; U.S. Pat. No. 5,693,622, 5,705,151, 5,580,859; Tabata H. et al. (1997) Cardiovasc. Res. 35(3):470-479, Chao J et al. (1997) Pharmacol. Res. 35(6):517-522, Wolff J. A. (1997) Neuromuscul. Disord. 7(5):314-318, Schwartz B. et al. (1996) Gene Ther. 3(5):405-411, Tsurumi Y. et al. (1996) Circulation 94(12):3281-3290 (incorporated herein by reference).

The FGFR5 polynucleotide constructs may be delivered by any method that delivers injectable materials to the cells of an animal, such as, injection into the interstitial space of tissues (heart, muscle, skin, lung, liver, intestine and the like). The FGFR5 polynucleotide constructs can be delivered in a pharmaceutically acceptable liquid or aqueous carrier.

The term “naked” polynucleotide, DNA or RNA, refers to sequences that are free from any delivery vehicle that acts to assist, promote, or facilitate entry into the cell, including viral sequences, viral particles, liposome formulations, lipofectin or precipitating agents and the like. However, the FGFR5 polynucleotides may also be delivered in liposome formulations (such as those taught in Felgner P. L. et al. (1995) Ann. NY Acad. Sci. 772:126-139 and Abdallah B. et al. (1995) Biol. Cell 85(1):1-7) which can be prepared by methods well known to those skilled in the art.

The FGFR5 polynucleotide vector constructs used in the gene therapy method are preferably constructs that will not integrate into the host genome nor will they contain sequences that allow for replication. Any strong promoter known to those skilled in the art can be used for driving the expression of DNA. Unlike other gene therapies techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The FGFR5 polynucleotide construct can be delivered to the interstitial space of tissues within the an animal, including of pancreas, kidney, muscle, skeletal muscle, skin, brain, lung, liver, spleen, bone marrow, thymus, heart, lymph, blood, bone, cartilage, gall bladder, stomach, intestine, testis, ovary, uterus, rectum, nervous system, eye, gland, and connective tissue. Interstitial space of the tissues comprises the intercellular fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked FGFR5 polynucleotide injection, an effective dosage amount of DNA or RNA will be in the range of from about 0.05 g/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and may depend on the condition being treated and the route of administration. The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked FGFR5 polynucleotide constructs can be delivered to arteries during angioplasty by the catheter used in the procedure.

The dose response effects of injected FGFR5 polynucleotide in muscle in vivo is determined as follows. Suitable FGFR5 template DNA for production of mRNA coding for FGFR5 polypeptide is prepared in accordance with a standard recombinant DNA methodology. The template DNA, which may be either circular or linear, is either used as naked DNA or complexed with liposomes. The quadriceps muscles of mice are then injected with various amounts of the template DNA.

Five to six week old female and male Balb/C mice are anesthetized by intraperitoneal injection with 0.3 ml of 2.5% Avertin. A 1.5 cm incision is made on the anterior thigh, and the quadriceps muscle is directly visualized. The FGFR5 template DNA is injected in 0.1 ml of carrier in a 1 cc syringe through a 27 gauge needle over one minute, approximately 0.5 cm from the distal insertion site of the muscle into the knee and about 0.2 cm deep. A suture is placed over the injection site for future localization, and the skin is closed with stainless steel clips.

After an appropriate incubation time (e.g., 7 days) muscle extracts are prepared by excising the entire quadriceps. Every fifth 15 um cross-section of the individual quadriceps muscles is histochemically stained for FGFR5 protein expression. A time course for FGFR5 protein expression may be done in a similar fashion except that quadriceps from different mice are harvested at different times. Persistence of FGFR5 DNA in muscle following injection may be determined by Southern blot analysis after preparing total cellular DNA and HIRT supernatants from injected and control mice. The results of the above experimentation in mice will be used to extrapolate proper dosages and other treatment parameters in humans and other animals using FGFR5 naked DNA.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, Detailed Description, and Examples is hereby incorporated herein by reference.

Moreover, the sequence submitted herewith in paper and computer readable form are herein incorporated by reference in their entireties.

### Example 17

### FGFR5 Transgenic Animals

The FGFR5 polypeptides can also be expressed in transgenic animals. Animals of any species, including, but not limited to, mice, rats, rabbits, hamsters, guinea pigs, pigs, micro-pigs, goats, sheep, cows and non-human primates, e.g., baboons, monkeys, and chimpanzees may be used to generate transgenic animals. In a specific embodiment, techniques described herein or otherwise known in the art, are used to express polypeptides of the invention in humans, as part of a gene therapy protocol.

Any technique known in the art may be used to introduce the transgene (i.e., polynucleotides of the invention) into animals to produce the founder lines of transgenic animals. Such techniques include, but are not limited to, pronuclear microinjection (Paterson et al., Appl. Microbiol. Biotechnol. 40:691-698 (1994); Carver et al., Biotechnology (NY) 11:1263-1270 (1993); Wright et al., Biotechnology (NY) 9:830-834 (1991); and Hoppe et al., U.S. Pat. No. 4,873,191 (1989)); retrovirus mediated gene transfer into germ lines (Van der Putten et al., Proc. Natl. Acad. Sci., USA 82:6148-6152 (1985)), blastocysts or embryos; gene targeting in embryonic stem cells (Thompson et al., Cell 56:313-321 (1989)); electroporation of cells or embryos (Lo, 1983, Mol Cell. Biol. 3:1803-1814 (1983)); introduction of the polynucleotides of the invention using a gene gun (see, e.g., Ulmer et al., Science 259:1745 (1993); introducing nucleic acid constructs into embryonic pleuripotent stem cells and transferring the stem cells back into the blastocyst; and sperm-mediated gene transfer (Lavitrano et al., Cell 57:717-723 (1989); etc. For a review of such techniques, see Gordon, “Transgenic Animals,” Intl. Rev. Cytol. 115:171-229 (1989), which is incorporated by reference herein in its entirety.

Any technique known in the art may be used to produce transgenic clones containing polynucleotides of the invention, for example, nuclear transfer into enucleated oocytes of nuclei from cultured embryonic, fetal, or adult cells induced to quiescence (Campell et al., Nature 380:64-66 (1996); Wilmut et al., Nature 385:810-813 (1997)).

The present invention provides for transgenic animals that carry the transgene in all their cells, as well as animals which carry the transgene in some, but not all their cells, i.e., mosaic animals or chimeric. The transgene may be integrated as a single transgene or as multiple copies such as in concatamers, e.g., head-to-head tandems or head-to-tail tandems. The transgene may also be selectively introduced into and activated in a particular cell type by following, for example, the teaching of Lasko et al. (Lasko et al., Proc. Natl. Acad. Sci. USA 89:6232-6236 (1992)). The regulatory sequences required for such a cell-type specific activation will depend upon the particular cell type of interest, and will be apparent to those of skill in the art. When it is desired that the polynucleotide transgene be integrated into the chromosomal site of the endogenous gene, gene targeting is preferred.

Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene. The transgene may also be selectively introduced into a particular cell type, thus inactivating the endogenous gene in only that cell type, by following, for example, the teaching of Gu et al. (Gu et al., Science 265:103-106 (1994)). The regulatory sequences required for such a cell-type specific inactivation will depend upon the particular cell type of interest, and will be apparent to those of skill in the art. The contents of each of the documents recited in this paragraph is herein incorporated by reference in its entirety.

Once transgenic animals have been generated, the expression of the recombinant gene may be assayed utilizing standard techniques. Initial screening may be accomplished by Southern blot analysis or PCR techniques to analyze animal tissues to verify that integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and reverse transcriptase-PCR (rt-PCR). Samples of transgenic gene-expressing tissue may also be evaluated immunocytochemically or immunohistochemically using antibodies specific for the transgene product.

Once the founder animals are produced, they may be bred, inbred, outbred, or crossbred to produce colonies of the particular animal. Examples of such breeding strategies include, but are not limited to: outbreeding of founder animals with more than one integration site in order to establish separate lines; inbreeding of separate lines in order to produce compound transgenics that express the transgene at higher levels because of the effects of additive expression of each transgene; crossing of heterozygous transgenic animals to produce animals homozygous for a given integration site in order to both augment expression and eliminate the need for screening of animals by DNA analysis; crossing of separate homozygous lines to produce compound heterozygous or homozygous lines; and breeding to place the transgene on a distinct background that is appropriate for an experimental model of interest.

Transgenic animals of the invention have uses which include, but are not limited to, animal model systems useful in elaborating the biological function of FGFR5 polypeptides, studying conditions and/or disorders associated with aberrant FGFR5 expression, and in screening for compounds effective in ameliorating such conditions and/or disorders.

### Example 18

### FGFR5 Knock-Out Animals

Endogenous FGFR5 gene expression can also be reduced by inactivating or “knocking out” the FGFR5 gene and/or its promoter using targeted homologous recombination. (E.g., see Smithies et al., Nature 317:230-234 (1985); Thomas & Capecchi, Cell 51:503-512 (1987); Thompson et al., Cell 5:313-321 (1989); each of which is incorporated by reference herein in its entirety). For example, a mutant, non-functional polynucleotide of the invention (or a completely unrelated DNA sequence) flanked by DNA homologous to the endogenous polynucleotide sequence (either the coding regions or regulatory regions of the gene) can be used, with or without a selectable marker and/or a negative selectable marker, to transfect cells that express polypeptides of the invention in vivo. In another embodiment, techniques known in the art are used to generate knockouts in cells that contain, but do not express the gene of interest. Insertion of the DNA construct, via targeted homologous recombination, results in inactivation of the targeted gene. Such approaches are particularly suited in research and agricultural fields where modifications to embryonic stem cells can be used to generate animal offspring with an inactive targeted gene (e.g., see Thomas & Capecchi 1987 and Thompson 1989, supra). However this approach can be routinely adapted for use in humans provided the recombinant DNA constructs are directly administered or targeted to the required site in vivo using appropriate viral vectors that will be apparent to those of skill in the art.

In further embodiments of the invention, cells that are genetically engineered to express the polypeptides of the invention, or alternatively, that are genetically engineered not to express the polypeptides of the invention (e.g., knockouts) are administered to a patient in vivo. Such cells may be obtained from the patient (i.e., animal, including human) or an MHC compatible donor and can include, but are not limited to fibroblasts, bone marrow cells, blood cells (e.g., lymphocytes), adipocytes, muscle cells, endothelial cells etc. The cells are genetically engineered in vitro using recombinant DNA techniques to introduce the coding sequence of polypeptides of the invention into the cells, or alternatively, to disrupt the coding sequence and/or endogenous regulatory sequence associated with the polypeptides of the invention, e.g., by transduction (using viral vectors, and preferably vectors that integrate the transgene into the cell genome) or transfection procedures, including, but not limited to, the use of plasmids, cosmids, YACs, naked DNA, electroporation, liposomes, etc. The coding sequence of the polypeptides of the invention can be placed under the control of a strong constitutive or inducible promoter or promoter/enhancer to achieve expression, and preferably secretion, of the FGFR5 polypeptides. The engineered cells which express and preferably secrete the polypeptides of the invention can be introduced into the patient systemically, e.g., in the circulation, or intraperitoneally.

Alternatively, the cells can be incorporated into a matrix and implanted in the body, e.g., genetically engineered fibroblasts can be implanted as part of a skin graft; genetically engineered endothelial cells can be implanted as part of a lymphatic or vascular graft. (See, for example, Anderson et al. U.S. Pat. No. 5,399,349; and Mulligan & Wilson, U.S. Pat. No. 5,460,959 each of which is incorporated by reference herein in its entirety).

When the cells to be administered are non-autologous or non-MHC compatible cells, they can be administered using well known techniques which prevent the development of a host immune response against the introduced cells. For example, the cells may be introduced in an encapsulated form which, while allowing for an exchange of components with the immediate extracellular environment, does not allow the introduced cells to be recognized by the host immune system.

Knock-out animals of the invention have uses which include, but are not limited to, animal model systems useful in elaborating the biological function of FGFR5 polypeptides, studying conditions and/or disorders associated with aberrant FGFR5 expression, and in screening for compounds effective in ameliorating such conditions and/or disorders.

### Example 19

### Screening Assay Identifying Changes in Small Molecule Concentration and Membrane Permeability

Binding of a ligand to a receptor is known to alter intracellular levels of small molecules, such as calcium, potassium, sodium, and pH, as well as alter membrane potential. These alterations can be measured in an assay to identify supernatants which bind to receptors of a particular cell. Although the following protocol describes an assay for calcium, this protocol can easily be modified to detect changes in potassium, sodium, pH, membrane potential, or any other small molecule which is detectable by a fluorescent probe.

The following assay uses Fluorometric Imaging Plate Reader (“FLIPR”) to measure changes in fluorescent molecules (Molecular Probes) that bind small molecules. Clearly, any fluorescent molecule detecting a small molecule can be used instead of the calcium fluorescent molecule, fluo-3, used here.

For adherent cells, seed the cells at 10,000-20,000 cells/well in a Co-star black 96-well plate with clear bottom. The plate is incubated in a C0₂ incubator for 20 hours. The adherent cells are washed two times in Biotek washer with 200 ul of HBSS (Hank's Balanced Salt Solution) leaving 100 ul of buffer after the final wash.

A stock solution of 1 mg/ml fluo-3 is made in 10% pluronic acid DMSO. To load the cells with fluo-3, 50 ul of 12 ug/ml fluo-3 is added to each well. The plate is incubated at 37 degree C. in a CO₂ incubator for 60 min. The plate is washed four times in the Biotek washer with HBSS leaving 100 ul of buffer.

For non-adherent cells, the cells are spun down from culture media. Cells are re-suspended to 2-5×10⁶ cells/ml with HBSS in a 50-ml conical tube. 4 ul of 1 mg/ml fluo-3 solution in 10% pluronic acid DMSO is added to each ml of cell suspension. The tube is then placed in a 37 degree C. water bath for 30-60 min. The cells are washed twice with HBSS, resuspended to 1×10⁶ cells/ml, and dispensed into a microplate, 100 ul/well. The plate is centrifuged at 1000 rpm for 5 min. The plate is then washed once in Denley CellWash with 200 ul, followed by an aspiration step to 100 ul final volume.

For a non-cell based assay, each well contains a fluorescent molecule, such as fluo-3. The supernatant is added to the well, and a change in fluorescence is detected.

To measure the fluorescence of intracellular calcium, the FLIPR is set for the following parameters: (1) System gain is 300-800 mW; (2) Exposure time is 0.4 second; (3) Camera F/stop is F/2; (4) Excitation is 488 nm; (5) Emission is 530 nm; and (6) Sample addition is 50 ul. Increased emission at 530 nm indicates an extracellular signaling event caused by a molecule, either FGFR5 or a molecule that has binding specificity to FGFR5, such as a ligand, the binding of which has resulted in an increase in the intracellular Ca⁺⁺ concentration.

### Example 20

### Stimulation of Endothelial Migration

This example will be used to explore the possibility that FGFR5 may stimulate lymphatic endothelial cell migration.

Endothelial cell migration assays are performed using a 48 well microchemotaxis chamber (Neuroprobe Inc., Cabin John, MD; Falk, W., Goodwin, R. H. J., and Leonard, E. J. “A 48 well micro chemotaxis assembly for rapid and accurate measurement of leukocyte migration.” J. Immunological Methods 1980;33:239-247). Polyvinylpyrrolidone-free polycarbonate filters with a pore size of 8 um (Nucleopore Corp. Cambridge, Mass.) are coated with 0.1% gelatin for at least 6 hours at room temperature and dried under sterile air. Test substances are diluted to appropriate concentrations in M199 supplemented with 0.25% bovine serum albumin (BSA), and 25 ul of the final dilution is placed in the lower chamber of the modified Boyden apparatus. Subconfluent, early passage (2-6) HUVEC or BMEC cultures are washed and trypsinized for the minimum time required to achieve cell detachment. After placing the filter between lower and upper chamber, 2.5×10⁵ cells suspended in 50 ul M199 containing 1% FBS are seeded in the upper compartment. The apparatus is then incubated for 5 hours at 37° C. in a humidified chamber with 5% CO2 to allow cell migration. After the incubation period, the filter is removed and the upper side of the filter with the non-migrated cells is scraped with a rubber policeman. The filters are fixed with methanol and stained with a Giemsa solution (Diff-Quick, Baxter, McGraw Park, Ill.). Migration is quantified by counting cells of three random high-power fields (40×) in each well, and all groups are performed in quadruplicate.

The studies described in this example tested activity in FGFR5 protein. However, one skilled in the art could easily modify the exemplified studies to test the activity of FGFR5 polynucleotides (e.g., gene therapy), agonists, and/or antagonists of FGFR5.

### Example 21

### Stimulation of Nitric Oxide Production by Endothelial Cells

Nitric oxide released by the vascular endothelium is believed to be a mediator of vascular endothelium relaxation. Thus, FGFR5 activity can be assayed by determining nitric oxide production by endothelial cells in response to FGFR5.

Nitric oxide is measured in 96-well plates of confluent microvascular endothelial cells after 24 hours starvation and a subsequent 4 hr exposure to various levels of a positive control (such as VEGF-1) and FGFR5. Nitric oxide in the medium is determined by use of the Griess reagent to measure total nitrite after reduction of nitric oxide-derived nitrate by nitrate reductase. The effect of FGFR5 on nitric oxide release is examined on HUVEC.

Briefly, NO release from cultured HUVEC monolayer is measured with a NO-specific polarographic electrode connected to a NO meter (Iso-NO, World Precision Instruments Inc.) (1049). Calibration of the NO elements is performed according to the following equation:

2 KNO₂+2 KI+2 H₂SO₄ 6 2 NO+I₂+2H₂O+2K₂SO₄

The standard calibration curve is obtained by adding graded concentrations of KNO2 (0, 5, 10, 25, 50, 100, 250, and 500 nmol/L) into the calibration solution containing KI and H₂SO₄. The specificity of the Iso-NO electrode to NO is previously determined by measurement of NO from authentic NO gas (1050). The culture medium is removed and HUVECs are washed twice with Dulbecco's phosphate buffered saline. The cells are then bathed in 5 ml of filtered Krebs-Henseleit solution in 6-well plates, and the cell plates are kept on a slide warmer (Lab Line instruments Inc.) To maintain the temperature at 37° C. The NO sensor probe is inserted vertically into the wells, keeping the tip of the electrode 2 mm under the surface of the solution, before addition of the different conditions. S-nitroso acetyl penicillamin (SNAP) is used as a positive control. The amount of released NO is expressed as picomoles per 1×10⁶ endothelial cells. All values reported are means of four to six measurements in each group (number of cell culture wells). See, Leak et al. Biochem. and Biophys. Res. Comm. 217:96-105 (1995).

The studies described in this example tested activity in FGFR5 protein. However, one skilled in the art could easily modify the exemplified studies to test the activity of FGFR5 polynucleotides (e.g., gene therapy), agonists, and/or antagonists of FGFR5.

### Example 22

### Angiogenic Effect on Chick Chorioallantoic Membrane

Chick chorioallantoic membrane (CAM) is a well-established system to examine angiogenesis. Blood vessel formation on CAM is easily visible and quantifiable. The ability of FGFR5 to stimulate angiogenesis in CAM can be examined.

Fertilized eggs of the White Leghorn chick (Gallus gallus) and the Japanese qual (Coturnix coturnix) are incubated at 37.8° C. and 80% humidity. Differentiated CAM of 16-day-old chick and 13-day-old qual embryos is studied with the following methods.

On Day 4 of development, a window is made into the egg shell of chick eggs. The embryos are checked for normal development and the eggs sealed with cellotape. They are further incubated until Day 13. Thermanox coverslips (Nunc, Naperville, Ill.) are cut into disks of about 5 mm in diameter. Sterile and salt-free growth factors are dissolved in distilled water and about 3.3 mg/5 ml are pipetted on the disks. After air-drying, the inverted disks are applied on CAM. After 3 days, the specimens are fixed in 3% glutaraldehyde and 2% formaldehyde and rinsed in 0.12 M sodium cacodylate buffer. They are photographed with a stereo microscope [Wild M8] and embedded for semi- and ultrathin sectioning as described above. Controls are performed with carrier disks alone.

The studies described in this example tested activity in FGFR5 protein. However, one skilled in the art could easily modify the exemplified studies to test the activity of FGFR5 polynucleotides (e.g., gene therapy), agonists, and/or antagonists of FGFR5.

### Example 23

### Rescue of Ischemia in Rabbit Lower Limb Model

To study the in vivo effects of FGFR5 on ischemia, a rabbit hindlimb ischemia model is created by surgical removal of one femoral arteries as described previously (Takeshita, S. et al., Am J. Pathol 147:1649-1660 (1995)). The excision of the femoral artery results in retrograde propagation of thrombus and occlusion of the external iliac artery. Consequently, blood flow to the ischemic limb is dependent upon collateral vessels originating from the internal iliac artery (Takeshita, S. et al. Am J Pathol 147:1649-1660 (1995)). An interval of 10 days is allowed for post-operative recovery of rabbits and development of endogenous collateral vessels. At 10 day post-operatively (day 0), after performing a baseline angiogram, the internal iliac artery of the ischemic limb is transfected with 500 mg naked FGFR5 expression plasmid by arterial gene transfer technology using a hydrogel-coated balloon catheter as described (Riessen, R. et al. Hum Gene Ther. 4:749-758 (1993); Leclerc, G. et al. J. Clin. Invest. 90: 936-944 (1992)). When FGFR5 is used in the treatment, a single bolus of 500 mg FGFR5 protein or control is delivered into the internal iliac artery of the ischemic limb over a period of 1 min. through an infusion catheter. On day 30, various parameters are measured in these rabbits: (a) BP ratio—The blood pressure ratio of systolic pressure of the ischemic limb to that of normal limb; (b) Blood Flow and Flow Reserve—Resting FL: the blood flow during undilated condition and Max FL: the blood flow during fully dilated condition (also an indirect measure of the blood vessel amount) and Flow Reserve is reflected by the ratio of max FL: resting FL; (c) Angiographic Score—This is measured by the angiogram of collateral vessels. A score is determined by the percentage of circles in an overlaying grid that with crossing opacified arteries divided by the total number m the rabbit thigh; (d) Capillary density—The number of collateral capillaries determined in light microscopic sections taken from hindlimbs.

The studies described in this example tested activity in FGFR5 protein. However, one skilled in the art could easily modify the exemplified studies to test the activity of FGFR5 polynucleotides (e.g., gene therapy), agonists, and/or antagonists of FGFR5.

### Example 24

### Effect of FGFR5 on Vasodilation

Since dilation of vascular endothelium is important in reducing blood pressure, the ability of FGFR5 to affect the blood pressure in spontaneously hypertensive rats (SHR) is examined. Increasing doses (0, 10, 30, 100, 300, and 900 mg/kg) of the FGFR5 are administered to 13-14 week old spontaneously hypertensive rats (SHR). Data are expressed as the mean±SEM. Statistical analysis are performed with a paired t-test and statistical significance is defined as p<0.05 vs. the response to buffer alone.

The studies described in this example tested activity in FGFR5 protein. However, one skilled in the art could easily modify the exemplified studies to test the activity of FGFR5 polynucleotides (e.g., gene therapy), agonists, and/or antagonists of FGFR5.

### Example 25

### Rat Ischemic Skin Flap Model

The evaluation parameters include skin blood flow, skin temperature, and factor VIII immunohistochemistry or endothelial alkaline phosphatase reaction. FGFR5 expression, during the skin ischemia, is studied using in situ hybridization.

The study in this model is divided into three parts as follows:

a) Ischemic skin

b) Ischemic skin wounds

c) Normal wounds

The experimental protocol includes:

a) Raising a 3×4 cm, single pedicle full-thickness random skin flap (myocutaneous flap over the lower back of the animal).

b) An excisional wounding (4-6 mm in diameter) in the ischemic skin (skin-flap).

c) Topical treatment with FGFR5 of the excisional wounds (day 0, 1, 2, 3, 4 post-wounding) at the following various dosage ranges: 1 mg to 100 mg.

d) Harvesting the wound tissues at day 3, 5, 7, 10, 14 and 21 post-wounding for histological, immunohistochemical, and in situ studies.

The studies described in this example tested activity in FGFR5 protein. However, one skilled in the art could easily modify the exemplified studies to test the activity of FGFR5 polynucleotides (e.g., gene therapy), agonists, and/or antagonists of FGFR5.

### Example 26

### Peripheral Arterial Disease Model

Angiogenic therapy using FGFR5 is a novel therapeutic strategy to obtain restoration of blood flow around the ischemia in case of peripheral arterial diseases. The experimental protocol includes:

a) One side of the femoral artery is ligated to create ischemic muscle of the hindlimb, the other side of hindlimb serves as a control.

b) FGFR5 protein, in a dosage range of 20 mg-500 mg, is delivered intravenously and/or intramuscularly 3 times (perhaps more) per week for 2-3 weeks.

c) The ischemic muscle tissue is collected after ligation of the femoral artery at 1, 2, and 3 weeks for the analysis of FGFR5 expression and histology. Biopsy is also performed on the other side of normal muscle of the contralateral hindlimb.

The studies described in this example tested activity in FGFR5 protein. However, one skilled in the art could easily modify the exemplified studies to test the activity of FGFR5 polynucleotides (e.g., gene therapy), agonists, and/or antagonists of FGFR5.

### Example 27

### Ischemic Myocardial Disease Model

FGFR5 is evaluated as a potent mitogen capable of stimulating the development of collateral vessels, and restructuring new vessels after coronary artery occlusion. Alteration of FGFR5 expression is investigated in situ. The experimental protocol includes:

a) The heart is exposed through a left-side thoracotomy in the rat. Immediately, the left coronary artery is occluded with a thin suture (6-0) and the thorax is closed.

b) FGFR5 protein, in a dosage range of 20 mg-500 mg, is delivered intravenously and/or intramuscularly 3 times (perhaps more) per week for 2-4 weeks.

c) Thirty days after the surgery, the heart is removed and cross-sectioned for morphometric and in situ analyzes.

The studies described in this example tested activity in FGFR5 protein. However, one skilled in the art could easily modify the exemplified studies to test the activity of FGFR5 polynucleotides (e.g., gene therapy), agonists, and/or antagonists of FGFR5.

### Example 28

### Rat Corneal Wound Healing Model

This animal model shows the effect of FGFR5 on neovascularization. The experimental protocol includes:

a) Making a 1-1.5 mm long incision from the center of cornea into the stromal layer.

b) Inserting a spatula below the lip of the incision facing the outer corner of the eye.

c) Making a pocket (its base is 1-1.5 mm form the edge of the eye).

d) Positioning a pellet, containing 50 ng-5 ug of FGFR5, within the pocket.

e) FGFR5 treatment can also be applied topically to the corneal wounds in a dosage range of 20 mg-500 mg (daily treatment for five days).

The studies described in this example tested activity in FGFR5 protein. However, one skilled in the art could easily modify the exemplified studies to test the activity of FGFR5 polynucleotides (e.g., gene therapy), agonists, and/or antagonists of FGFR5.

### Example 29

### Diabetic Mouse and Glucocorticoid-Impaired Wound Healing Models

A. Diabetic db+/db+ Mouse Model

To demonstrate that FGFR5 accelerates the healing process, the genetically diabetic mouse model of wound healing is used. The full thickness wound healing model in the db+/db+ mouse is a well characterized, clinically relevant and reproducible model of impaired wound healing. Healing of the diabetic wound is dependent on formation of granulation tissue and re-epithelialization rather than contraction (Gartner, M. H. et al., J. Surg. Res. 52:389 (1992); Greenhalgh, D. G. et al., Am. J. Pathol. 136:1235 (1990)).

The diabetic animals have many of the characteristic features observed in Type II diabetes mellitus. Homozygous (db+/db+) mice are obese in comparison to their normal heterozygous (db+/+m) littermates. Mutant diabetic (db+/db+) mice have a single autosomal recessive mutation on chromosome 4 (db+) (Coleman et al. Proc. Natl. Acad. Sci. USA 77:283-293 (1982)). Animals show polyphagia, polydipsia and polyuria. Mutant diabetic mice (db+/db+) have elevated blood glucose, increased or normal insulin levels, and suppressed cell-mediated immunity (Mandel et al., J. Immunol. 120:1375 (1978); Debray-Sachs, M. et al., Clin. Exp. Immunol. 51(1):1-7 (1983); Leiter et al., Am. J. of Pathol. 114:46-55 (1985)). Peripheral neuropathy, myocardial complications, and microvascular lesions, basement membrane thickening and glomerular filtration abnormalities have been described in these animals (Norido, F. et al., Exp. Neurol. 83(2):221-232 (1984); Robertson et al., Diabetes 29(1):60-67 (1980); Giacomelli et al., Lab Invest. 40(4):460-473 (1979); Coleman, D. L., Diabetes 31 (Suppl):1-6 (1982)). These homozygous diabetic mice develop hyperglycemia that is resistant to insulin analogous to human type II diabetes (Mandel et al., J. Immunol. 120:1375-1377 (1978)).

The characteristics observed in these animals suggests that healing in this model may be similar to the healing observed in human diabetes (Greenhalgh, et al., Am. J. of Pathol. 136:1235-1246 (1990)).

Genetically diabetic female C57BL/KsJ (db+/db+) mice and their non-diabetic (db+/+m) heterozygous littermates are used in this study (Jackson Laboratories). The animals are purchased at 6 weeks of age and were 8 weeks old at the beginning of the study. Animals are individually housed and received food and water ad libitum. All manipulations are performed using aseptic techniques. The experiments are conducted according to the rules and guidelines of Human Genome Sciences, Inc. Institutional Animal Care and Use Committee and the Guidelines for the Care and Use of Laboratory Animals.

Wounding protocol is performed according to previously reported methods (Tsuboi, R. and Rifkin, D. B., J. Exp. Med. 172:245-251 (1990)). Briefly, on the day of wounding, animals are anesthetized with an intraperitoneal injection of Avertin (0.01 mg/mL), 2,2,2-tribromoethanol and 2-methyl-2-butanol dissolved in deionized water. The dorsal region of the animal is shaved and the skin washed with 70% ethanol solution and iodine. The surgical area is dried with sterile gauze prior to wounding. An 8 mm full-thickness wound is then created using a Keyes tissue punch. Immediately following wounding, the surrounding skin is gently stretched to eliminate wound expansion. The wounds are left open for the duration of the experiment. Application of the treatment is given topically for 5 consecutive days commencing on the day of wounding. Prior to treatment, wounds are gently cleansed with sterile saline and gauze sponges.

Wounds are visually examined and photographed at a fixed distance at the day of surgery and at two day intervals thereafter. Wound closure is determined by daily measurement on days 1-5 and on day 8. Wounds are measured horizontally and vertically using a calibrated Jameson caliper. Wounds are considered healed if granulation tissue is no longer visible and the wound is covered by a continuous epithelium.

FGFR5 is administered using at a range different doses of FGFR5, from 4 mg to 500 mg per wound per day for 8 days in vehicle. Vehicle control groups received 50 mL of vehicle solution.

Animals are euthanized on day 8 with an intraperitoneal injection of sodium pentobarbital (300 mg/kg). The wounds and surrounding skin are then harvested for histology and immunohistochemistry. Tissue specimens are placed in 10% neutral buffered formalin in tissue cassettes between biopsy sponges for further processing.

Three groups of 10 animals each (5 diabetic and 5 non-diabetic controls) are evaluated: 1) Vehicle placebo control, 2) FGFR5.

Wound closure is analyzed by measuring the area in the vertical and horizontal axis and obtaining the total square area of the wound. Contraction is then estimated by establishing the differences between the initial wound area (day 0) and that of post treatment (day 8). The wound area on day 1 was 64 mm², the corresponding size of the dermal punch. Calculations were made using the following formula:

[Open area on day 8]−[Open area on day 1]/[Open area on day 1]

Specimens are fixed in 10% buffered formalin and paraffin embedded blocks are sectioned perpendicular to the wound surface (5mm) and cut using a Reichert-Jung microtome. Routine hematoxylin-eosin (H&E) staining is performed on cross-sections of bisected wounds. Histologic examination of the wounds are used to assess whether the healing process and the morphologic appearance of the repaired skin is altered by treatment with FGFR5. This assessment included verification of the presence of cell accumulation, inflammatory cells, capillaries, fibroblasts, re-epithelialization and epidermal maturity (Greenhalgh, D. G. et al., Am. J. Pathol. 136:1235 (1990)). A calibrated lens micrometer is used by a blinded observer.

Tissue sections are also stained immunohistochemically with a polyclonal rabbit anti-human keratin antibody using ABC Elite detection system. Human skin is used as a positive tissue control while non-immune IgG is used as a negative control. Keratinocyte growth is determined by evaluating the extent of reepithelialization of the wound using a calibrated lens micrometer.

Proliferating cell nuclear antigen/cyclin (PCNA) in skin specimens is demonstrated by using anti-PCNA antibody (1:50) with an ABC Elite detection system. Human colon cancer served as a positive tissue control and human brain tissue is used as a negative tissue control. Each specimen included a section with omission of the primary antibody and substitution with non-immune mouse IgG. Ranking of these sections is based on the extent of proliferation on a scale of 0-8, the lower side of the scale reflecting slight proliferation to the higher side reflecting intense proliferation.

Experimental data are analyzed using an unpaired t test. A p value of <0.05 is considered significant.

B. Steroid Impaired Rat Model

The inhibition of wound healing by steroids has been well documented in various in vitro and in vivo systems (Wahl, S. M. Glucocorticoids and Wound healing. In: Anti-Inflammatory Steroid Action: Basic and Clinical Aspects. 280-302 (1989); Wahl, S. M.et al., J. Immunol. 115: 476-481 (1975); Werb, Z. et al., J. Exp. Med. 147:1684-1694 (1978)). Glucocorticoids retard wound healing by inhibiting angiogenesis, decreasing vascular permeability (Ebert, R. H., et al., An. Intern. Med. 37:701-705 (1952)), fibroblast proliferation, and collagen synthesis (Beck, L. S. et al., Growth Factors. 5: 295-304 (1991); Haynes, B. F. et al., J. Clin. Invest. 61: 703-797 (I 978)) and producing a transient reduction of circulating monocytes (Haynes, B. F., et al., J. Clin. Invest. 61: 703-797 (1978); Wahl, S. M., “Glucocorticoids and wound healing”, In: Antiinflammatory Steroid Action: Basic and Clinical Aspects, Academic Press, New York, pp. 280-302 (1989)). The systemic administration of steroids to impaired wound healing is a well establish phenomenon in rats (Beck, L. S. et al., Growth Factors. 5: 295-304 (1991); Haynes, B. F., et al., J. Clin. Invest. 61: 703-797 (1978); Wahl, S. M., “Glucocorticoids and wound healing”, In: Antiinflammatory Steroid Action: Basic and Clinical Aspects, Academic Press, New York, pp. 280-302 (1989); Pierce, G. F. et al., Proc. Natl. Acad. Sci. USA 86: 2229-2233 (1989)).

To demonstrate that FGFR5 can accelerate the healing process, the effects of multiple topical applications of FGFR5 on full thickness excisional skin wounds in rats in which healing has been impaired by the systemic administration of methylprednisolone is assessed.

Young adult male Sprague Dawley rats weighing 250-300 g (Charles River Laboratories) are used in this example. The animals are purchased at 8 weeks of age and were 9 weeks old at the beginning of the study. The healing response of rats is impaired by the systemic administration of methylprednisolone (17 mg/kg/rat intramuscularly) at the time of wounding. Animals are individually housed and received food and water ad libitum. All manipulations are performed using aseptic techniques. This study is conducted according to the rules and guidelines of Human Genome Sciences, Inc. Institutional Animal Care and Use Committee and the Guidelines for the Care and Use of Laboratory Animals.

The wounding protocol is followed according to section A, above. On the day of wounding, animals are anesthetized with an intramuscular injection of ketamine (50 mg/kg) and xylazine (5 mg/kg). The dorsal region of the animal is shaved and the skin washed with 70% ethanol and iodine solutions. The surgical area is dried with sterile gauze prior to wounding. An 8 mm full-thickness wound is created using a Keyes tissue punch. The wounds are left open for the duration of the experiment. Applications of the testing materials are given topically once a day for 7 consecutive days commencing on the day of wounding and subsequent to methylprednisolone administration. Prior to treatment, wounds are gently cleansed with sterile saline and gauze sponges.

Wounds are visually examined and photographed at a fixed distance at the day of wounding and at the end of treatment. Wound closure is determined by daily measurement on days 1-5 and on day 8 for Figure. Wounds are measured horizontally and vertically using a calibrated Jameson caliper. Wounds are considered healed if granulation tissue was no longer visible and the wound is covered by a continuous epithelium.

FGFR5 is administered using at a range different doses of FGFR5, from 4 mg to 500 mg per wound per day for 8 days in vehicle. Vehicle control groups received 50 mL of vehicle solution.

Animals are euthanized on day 8 with an intraperitoneal injection of sodium pentobarbital (300 mg/kg). The wounds and surrounding skin are then harvested for histology. Tissue specimens are placed in 10% neutral buffered formalin in tissue cassettes between biopsy sponges for further processing.

Four groups of 10 animals each (5 with methylprednisolone and 5 without glucocorticoid) were evaluated: 1) Untreated group 2) Vehicle placebo control 3) FGFR5 treated groups.

Wound closure is analyzed by measuring the area in the vertical and horizontal axis and obtaining the total area of the wound. Closure is then estimated by establishing the differences between the initial wound area (day 0) and that of post treatment (day 8). The wound area on day 1 was 64 mm², the corresponding size of the dermal punch. Calculations were made using the following formula:

[Open area on day 8]−[Open area on day 1]/[Open area on day 1]

Specimens are fixed in 10% buffered formalin and paraffin embedded blocks are sectioned perpendicular to the wound surface (5 mm) and cut using an Olympus microtome. Routine hematoxylin-eosin (H&E) staining was performed on cross-sections of bisected wounds. Histologic examination of the wounds allows assessment of whether the healing process and the morphologic appearance of the repaired skin was improved by treatment with FGFR5. A calibrated lens micrometer is used by a blinded observer to determine the distance of the wound gap.

Experimental data are analyzed using an unpaired t test. A p value of <0.05 is considered significant.

The studies described in this example tested activity in FGFR5 protein. However, one skilled in the art could easily modify the exemplified studies to test the activity of FGFR5 polynucleotides (e.g., gene therapy), agonists, and/or antagonists of FGFR5.

### Example 30

### Lymphadema Animal Model

The purpose of this experimental approach is to create an appropriate and consistent lymphedema model for testing the therapeutic effects of FGFR5 in lymphangiogenesis and re-establishment of the lymphatic circulatory system in the rat hind limb. Effectiveness is measured by swelling volume of the affected limb, quantification of the amount of lymphatic vasculature, total blood plasma protein, and histopathology. Acute lymphedema is observed for 7-10 days. Perhaps more importantly, the chronic progress of the edema is followed for up to 3-4 weeks.

Prior to beginning surgery, blood sample is drawn for protein concentration analysis. Male rats weighing approximately ˜350 g are dosed with Pentobarbital. Subsequently, the right legs are shaved from knee to hip. The shaved area is swabbed with gauze soaked in 70% EtOH. Blood is drawn for serum total protein testing. Circumference and volumetric measurements are made prior to injecting dye into paws after marking 2 measurement levels (0.5 cm above heel, at mid-pt of dorsal paw). The intradermal dorsum of both right and left paws are injected with 0.05 ml of 1% Evan's Blue. Circumference and volumetric measurements are then made following injection of dye into paws.

Using the knee joint as a landmark, a mid-leg inguinal incision is made circumferentially allowing the femoral vessels to be located. Forceps and hemostats are used to dissect and separate the skin flaps. After locating the femoral vessels, the lymphatic vessel that runs along side and underneath the vessel(s) is located. The main lymphatic vessels in this area are then electrically coagulated or suture ligated.

Using a microscope, muscles in back of the leg (near the semitendinosis and adductors) are bluntly dissected. The popliteal lymph node is then located. The 2 proximal and 2 distal lymphatic vessels and distal blood supply of the popliteal node are then and ligated by suturing. The popliteal lymph node, and any accompanying adipose tissue, is then removed by cutting connective tissues.

Care is taken to control any mild bleeding resulting from this procedure. After lymphatics are occluded, the skin flaps are sealed by using liquid skin (Vetbond) (A J Buck). The separated skin edges are sealed to the underlying muscle tissue while leaving a gap of ˜0.5 cm around the leg. Skin also may be anchored by suturing to underlying muscle when necessary.

To avoid infection, animals are housed individually with mesh (no bedding). Recovering animals are checked daily through the optimal edematous peak, which typically occurred by day 5-7. The plateau edematous peak are then observed. To evaluate the intensity of the lymphedema, the circumference and volumes of 2 designated places on each paw before operation and daily for 7 days are measured. The effect plasma proteins on lymphedema is determined and whether protein analysis is a useful testing perimeter is also investigated. The weights of both control and edematous limbs are evaluated at 2 places. Analysis is performed in a blind manner.

Circumference Measurements: Under brief gas anesthetic to prevent limb movement, a cloth tape is used to measure limb circumference. Measurements are done at the ankle bone and dorsal paw by 2 different people then those 2 readings are averaged. Readings are taken from both control and edematous limbs.

Volumetric Measurements: On the day of surgery, animals are anesthetized with Pentobarbital and are tested prior to surgery. For daily volumetrics animals are under brief halothane anesthetic (rapid immobilization and quick recovery), both legs are shaved and equally marked using waterproof marker on legs. Legs are first dipped in water, then dipped into instrument to each marked level then measured by Buxco edema software(Chen/Victor). Data is recorded by one person, while the other is dipping the limb to marked area.

Blood-plasma protein measurements: Blood is drawn, spun, and serum separated prior to surgery and then at conclusion for total protein and Ca2+ comparison.

Limb Weight Comparison: After drawing blood, the animal is prepared for tissue collection. The limbs were amputated using a quillitine, then both experimental and control legs were cut at the ligature and weighed. A second weighing is done as the tibio-cacaneal joint was disarticulated and the foot was weighed.

Histological Preparations: The transverse muscle located behind the knee (popliteal) area is dissected and arranged in a metal mold, filled with freezeGel, dipped into cold methylbutane, placed into labeled sample bags at −80EC until sectioning. Upon sectioning, the muscle was observed under fluorescent microscopy for lymphatics. Other immuno/histological methods are currently being evaluated. The studies described in this example tested activity in FGFR5 protein. However, one skilled in the art could easily modify the exemplified studies to test the activity of FGFR5 polynucleotides (e.g., gene therapy), agonists, and/or antagonists of FGFR5.

Various modifications of the invention, in addition to those shown and described herein will become apparent to those skilled in the art from the foregoing description and accompanying drawings. Such modifications are intended to fall within the scope of the appended claims.

The entire disclosure of each document cited (including patents, patent applications, journal articles, abstracts, laboratory manuals, books, or other disclosures) in the Background of the Invention, Detailed Description, and Examples is hereby incorporated herein by reference. Moreover, the sequence listing is herein incorporated by reference.

What is claimed is:

1. An isolated nucleic acid molecule comprising a polynucleotide having a nucleotide sequence at least 95% identical to a sequence selected from the group consisting of:
(a) a polynucleotide fragment of SEQ ID NO:1 or a polynucleotide fragment of the cDNA sequence included in ATCC Deposit No: 203382;
(b) a polynucleotide encoding a polypeptide fragment of SEQ ID NO:2 or a polypeptide encoded by the cDNA sequence included in ATCC Deposit No: 203382;
(c) a polynucleotide encoding a polypeptide domain of SEQ ID NO:2 or a polypeptide domain encoded by the cDNA sequence included in ATCC Deposit No: 203382;
(d) a polynucleotide encoding a polypeptide epitope of SEQ ID NO:2 or a polypeptide epitope encoded by the cDNA sequence included in ATCC Deposit No: 203382;
(e) a polynucleotide encoding a polypeptide of SEQ ID NO:2 or a polypeptide encoded by the cDNA sequence included in ATCC Deposit No: 203382, having biological activity; and
(f) a polynucleotide capable of hybridizing under stringent conditions to any one of the polynucleotides specified in (a)-(e), wherein said polynucleotide does not hybridize under stringent conditions to a nucleic acid molecule having a nucleotide sequence of only A residues or of only T residues.

2. The isolated nucleic acid molecule of claim 1, wherein the polynucleotide fragment comprises a nucleotide sequence encoding FGFR5 (SEQ ID NO:2).

3. The isolated nucleic acid molecule of claim 1, wherein the polynucleotide fragment comprises a nucleotide sequence encoding the polypeptide encoded by the cDNA sequence included in ATCC Deposit No: 203382.

4. The isolated nucleic acid molecule of claim 1, wherein the polynucleotide fragment comprises the entire nucleotide sequence of SEQ ID NO:1 or the cDNA sequence included in ATCC Deposit No: 203382.

5. The isolated nucleic acid molecule of claim 2, wherein the nucleotide sequence comprises sequential nucleotide deletions of nucleotides encoding either the C-terminus or the N-terminus.

6. The isolated nucleic acid molecule of claim 3, wherein the nucleotide sequence comprises sequential nucleotide deletions of nucleotides encoding either the C-terminus or the N-terminus.

7. A recombinant vector comprising the isolated nucleic acid molecule of claim 1.

8. A method of making a recombinant host cell comprising the isolated nucleic acid molecule of claim 1.

9. A recombinant host cell produced by the method of claim 8.

10. The recombinant host cell of claim 9 comprising vector sequences.

11. An isolated polypeptide comprising an amino acid sequence at least 95% identical to a sequence selected from the group consisting of:
(a) a polypeptide comprising the amino and sequence of SEQ ID NO:2 or the encoded sequence included in ATCC Deposit No: 203382;
(b) a polypeptide fragment of SEQ ID NO:2 or the encoded sequence included in ATCC Deposit No: 203382, having biological activity;
(c) a polypeptide domain of SEQ ID NO:2 or the encoded sequence included in ATCC Deposit No: 203382; and
(d) a polypeptide epitope of SEQ ID NO:2 or the encoded sequence included in ATCC Deposit No: 203382.

12. The isolated polypeptide of claim 11, wherein the polypeptide fragment comprises sequential amino acid deletions from either the C-terminus or the N-terminus.

13. An isolated antibody that binds specifically to the isolated polypeptide of claim 11.

14. A recombinant host cell that expresses the isolated polypeptide of claim 11.

15. A method of making an isolated polypeptide comprising:
(a) culturing the recombinant host cell of claim 14 under conditions such that said polypeptide is expressed; and
(b) recovering said polypeptide.

16. The polypeptide produced by claim 15.

17. A method for preventing, treating, or ameliorating a medical condition which comprises administering to a mammalian subject a therapeutically effective amount of the polypeptide of claim 11 or of the polynucleotide of claim 1.

18. A method of diagnosing a pathological condition or a susceptibility to a pathological condition in a subject related to expression or activity of FGFR5 comprising:
(a) determining the presence or absence of a mutation in the polynucleotide of claim 1;
(b) diagnosing a pathological condition or a susceptibility to a pathological condition based on the presence or absence of said mutation.

19. A method of diagnosing a pathological condition or a susceptibility to a pathological condition in a subject related to expression or activity of FGFR5 comprising:
(a) determining the presence or amount of expression of the polypeptide of claim 11 in a biological sample;
(b) diagnosing a pathological condition or a susceptibility to a pathological condition based on the presence or amount of expression of the polypeptide.

20. A method of identifying a binding partner to the polypeptide of claim 11 comprising:
(a) contacting the polypeptide of claim 11 with a plurality of compounds; and
(b) identifying compounds that bind the polypeptide.

21. A method of identifying compounds capable of enhancing or inhibiting a cellular response induced by FGFR5 comprising:
(a) contacting cells which express the polypeptide of claim 11, with a candidate compound; and
(b) assaying a cellular response.

22. The method of claim 21 wherein the cellular response is hydrolysis of polyphosphoinositides and/or ion flux.

23. The method of claim 21 wherein hydrolysis of polyphosphoinositides and/or cation flux is assayed.
